(19) United States
(12) Patent Application Publication
(10) Pub. No.: US 2025/0027289 A1
(43) Pub. Date: Jan. 23, 2025

(54) SELF-LEARNING FRAMEWORK FOR PREDICTIVE TOPOGRAPHIC MODELING AND INTELLIGENT REAL-TIME SYSTEM CONTROL OF SUBTERRANEAN SLURRY INJECTION SYSTEM

(71) Applicants: Cole Brayton Johnson, Atlanta, GA (US); Laurence E. Allen, III, San Rafael, CA (US); Laurence E. Allen, IV, San Rafael, CA (US)

(72) Inventors: Cole Brayton Johnson, Atlanta, GA (US); Laurence E. Allen, III, San Rafael, CA (US); Laurence E. Allen, IV, San Rafael, CA (US)

(21) Appl. No.: 18/629,026

(22) Filed: Apr. 8, 2024

Related U.S. Application Data
(63) Continuation-in-part of application No. 17/672,553, filed on Feb. 15, 2022, now Pat. No. 11,952,735.

(51) Int. Cl. E02D 3/12; G05B 13/02; G05B 13/04

(52) U.S. Cl. CPC E02D 3/12; G05B 13/0265; G05B 13/04

(57) ABSTRACT

The invention presents a system for geospatial topographical modeling and real-time system control to be used with subterranean slurry injection technology. The system consists of a predictive modeling subsystem, which processes land characteristics to generate optimal site plans for drilling and injection. The real-time control subsystem implements these plans, dynamically adjusting slurry compositions and injection parameters based on sensor feedback. A feedback loop between the subsystems allows continuous refinement of both predictive models and operational controls, enhancing the precision with which the technology shapes the ground and the efficiency with which the technology sequesters carbon.

### FEDERALLY SPONSORED RESEARCH

Not Applicable

### SEQUENCE LISTING OR PROGRAM

Not Applicable

### TECHNICAL FIELD OF THE INVENTION

The present disclosure is generally related to a method and systems for elevating areas. More particularly, the present disclosure relates to systems and methods for the subterranean injection of solids that are of biological origin including divided wood, algae and plant material for example saw dust, wood chips, trimmings, leaves, and grasses. In addition, the present disclosure refers generally to novel prediction and control frameworks for planning and operating subterranean slurry injection systems.

### BACKGROUND OF THE INVENTION

It is generally understood that sea levels will rise in conjunction with increased storm frequency and intensity as a result of elevated atmospheric CO2 levels. Certain coastal areas will face inundation by rising waters. There are few attractive options to protect coastal areas, though there are several understood techniques with varying levels of cost and protection. Common techniques include building dikes to exclude seawater and using pumps to drain sub-sea level areas of accumulated rain. This approach is used in a number of areas including the Netherlands and New Orleans. Other techniques include elevation of buildings and highways onto piers or surface fill.

Injecting solid material below the surface of the ground creates an option for elevating areas without substantial disturbance of existing constructions and infrastructure on the surface.

This is an attractive alternative because it avoids construction costs associated with moving or elevating buildings or roadways onto piers or surface fill. It also is a permanent solution that does not require maintenance or create risks of catastrophic inundation that areas below sea or river level behind dikes must endure.

An advantage of elevation of terrain and structures using subterranean injection of solids is that there is no disturbance of the actual use or characteristics of the existing structures when elevation or subterranean mechanical enhancement is done. Additionally, structures can be elevated a little at a time as needed once the rate of sea-level rise is understood or more accurately predicted. This spreads the cost of protecting structures over potentially many years rather than requiring that the entire cost be borne at one time.

Additionally, no final commitment to a given level of elevation must be made in advance of good knowledge of required future elevation. A little elevation at a time with the option to repeat the process with more elevation in the future as needed is a better approach. If there is compaction, settling, subsidence or decomposition of some portion of solids, more may be slowly added later as compensation. All data gathered can be applied to the modeling and procedural control of elevation, moreover.

Computational advancements that we have developed significantly enhance the ability to accurately model geospatial terrain, potentially now lending themselves to a crucial role in flood defense strategies. Modern geographic information systems and advanced simulation software enable precise mapping and simulation, therefore being instrumental in predicting how areas will respond to sea-level rise and storm surges. By using these high-resolution data and sophisticated modeling techniques, it's possible using this novel process to identify vulnerable zones and assess the impact of various mitigation strategies, such as elevation or dike construction.

In addition to terrain modeling, We have developed the computational ability to model elevation and uplift using techniques such as Computational Fluid Dynamics (CFD), Finite Element Analysis (FEA), and machine learning (ML) approaches which open new possibilities in property protection. CFD can simulate the interaction between water and structures, providing insights into the effectiveness of barriers or elevated areas under different scenarios. FEA helps in understanding the structural integrity of proposed solutions under various stress conditions. ML algorithms, meanwhile, can predict long-term outcomes by analyzing vast amounts of data. This invention combines these technologies with inventive algorithms to collectively enable a more informed, dynamic approach to designing and executing elevation procedures, potentially for the purpose of flood defense.

### Definitions

Lignocellulosic material, when used herein, is a short-hand for any biomass material or lignocellulosic material and is understood to be plant material and explicitly includes up to 100% leaves, grass-trimmings, wood pulp, rice-husks, corn stover or any plant-based product including wood ash and biochar which have undergone reactive processing.

Pyrolysis is a form of reactive processing employing application of heat and, similar to combustion, may yield more carbon-rich solid materials such as carbon-containing ash or more concentrated carbon-containing materials sometimes known as biochar.

Lignocellulosic material is meant as a short-hand for all photosynthesizing organisms and so is intended to also include phytoplankton and algae though these organisms do not necessarily synthesize cellulose or lignin. In some islands or coastal areas, the most ready source of plant materials available for subterranean injection may be algae.

By the term “substantially” it is meant that the recited characteristic, parameter, or value need not be achieved exactly, but that deviations or variations, including for example, tolerances, measurement error, measurement accuracy limitations and other factors known to those of ordinary skill in the art, may occur in amounts that do not preclude the effect the characteristic was intended to provide.

The term “wood chips” is used as a shorthand for any comparable biomass based material with a fibrous nature.

The term “height” is occasionally used to describe the least of three dimensions of an aperture, for example. It is not meant to restrictively be applied to altitude or dimension normal to the surface of the ground but rather is intended to indicate the smallest of three dimensions of a three dimensional shape. The height of a vertical aperture may thus project horizontal to the surface of the earth in this sense. The height is also meant to indicate the space between two surfaces. Because the surfaces will usually be somewhat curved, the height at one part of an aperture will point in one direction while the height at another part of the aperture with a different orientation will point in a different direction.

The term “geospatial,” as used herein, refers to a combination of subterranean and surface topographies.

The term “elevation,” as used herein, refers to the process of augmenting surface topography.

### SUMMARY OF THE INVENTION

The present disclosure relates to an apparatus and process to protect structures and terrain from inundation as well as to gain potential improvements in seismic performance during earth tremors. Expansion of terrain or island formation is also enabled by the systems and methods disclosed herein. Aspects of the disclosed systems and methods include selection of depth, spacing and diameter of holes to be drilled. Other aspects of the disclosed system include selection, formulation, preparation, concentration and injection of lignocellulosic-based slurries into subterranean spaces. Measurement and adjustment of surface altitude, site monitoring and the techniques used to achieve desired final surface topography are also important aspects. The apparatus used to achieve these goals is an additional aspect of the present disclosure.

An objective of the present disclosure is to reduce the cost of elevating terrain, earthworks, structures of every description including roadways, bridges, buildings, and homes with little or no damage or cost of reconstruction. Terrain may be expanded, and new islands may be formed where previously no dry land existed. An additional object of the present disclosure is to gain additional valuable benefits relative to the use of mineral solids or sediments as described in Germanovich and Murdoch.

In addition to protecting structures and terrain the subterranean injection of lignocellulosic material may gain advantages such as the alteration of the mechanical character of the ground to improve seismic performance. Such injection may offer protection against hazards the lignocellulosic material might otherwise pose such as risk of fire or decomposition to release atmospheric pollutants such as methane, nitrous oxide, carbon dioxide or noxious odors. Lignocellulosic material may also be injected into a subterranean space to provide a space to accept fluid or gas.

According to an exemplary arrangement, a method for altering a characteristic of the ground comprises the steps of preparing a lignocellulosic material, suspending the lignocellulosic material in a slurry to create a lignocellulosic slurry, creating a fluid movement of the lignocellulosic slurry, resuspending a portion of the lignocellulosic slurry with the fluid movement, and injecting the lignocellulosic slurry below a surface of the ground. This process may be repeated any number of times.

In one arrangement, the lignocellulosic material comprises a buoyant force on the order of approximately +/−0.2 g/cc or less.

In one arrangement, the lignocellulosic material comprises an intrinsic particle density of approximately 0.8 to about 1.2 g/cc.

In one arrangement, the lignocellulosic material comprises a molecular density of approximately 1.45 to about 1.55 g/cc.

In one arrangement, the lignocellulosic material is selected from a group consisting of saw dust, divided wood, plant material, wood chips, wood pulp, rice husks, corn stover, wood ash, biochar, trimmings, leaves, grasses, grass trimmings, phytoplankton, algae, and biomass materials.

According to another exemplary arrangement, a method of subterranean injection of lignocellulosic material comprises the steps of selecting a suitable location for terrain protection, accomplishing surface elevation documentation, and placing surface elevation and inclination change sensors on a surface.

The method of the present invention further comprises the steps of determining a desired depth of prospective subterranean solids, determining a desired orientation of prospective subterranean solids, determining at least one subterranean injection location, and creating an injection well to enable a transfer of solids from the surface to the determined desired depth of the prospective subterranean solid.

The method of the present invention further comprises the steps of creating a subterranean aperture by injecting fluid under pressure into the subterranean space, and injecting lignocellulosic material into the aperture by injection of an aqueous slurry.

The features, functions, and advantages can be achieved independently in various embodiments of the present disclosure or may be combined in yet other embodiments in which further details can be seen with reference to the following description and drawings.

Additionally, the present invention comprises a novel dual-faceted topographical predictive modeling and real-time subterranean slurry injection control system. The predictive modeling subsystem may consist of a ML based framework which takes as input numerous land-related characteristics and outputs an optimal site plan which may consist of recommendations regarding drilling and injection procedures. The real-time control subsystem may consist of processes to receive and execute suggested site plans, dynamically adjusting slurry compositions and injection parameters in accordance with sensor feedback. In preferred embodiments, the various elements of the system operate in a feedback loop where data collected from the real-time control system is used in conjunction with observed land characteristics to fine-tune the predictive modeling subsystem and the various models within the real-time control system itself.

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

FIG. 1 illustrates a method for the protection of structures with subterranean injection of lignocellulosic solids;

FIG. 2 illustrates a method of pre-selecting lignocellulosic materials for injection;

FIG. 3 illustrates optional varieties of slurry formation apparatus;

FIG. 4 illustrates slurry placement technique option A wherein a single well aperture is expanded with fluid and subsequently filled with a single variety of lignocellulosic solids and then relaxed;

FIG. 5 illustrates slurry placement technique option B employing a dual well aperture and directional bulk flow with reverse flow sweeping cycle;

FIG. 6 illustrates slurry placement technique option C dual well aperture injection with material interchange to add a different variety of solids in reverse flow;

FIG. 7 illustrates a hypothetical resort uplift example;

FIG. 8 illustrates a hypothetical highway elevation project with bulk flow.

FIG. 9 illustrates two varieties of soil anchoring mechanisms that can apply compression to the soil.

FIG. 10 illustrates in more detail soil anchoring mechanisms that can apply compression to the soil as well as inject fluid into an area of the soil above the lower anchor.

FIG. 11 illustrates a soil anchor that can apply either compression or tension to an area of soil intermediate between two anchor sections

FIG. 12 illustrates how an array of soil anchoring devices may be applied in combination to alter the way in which a subterranean aperture is shaped, filled and compressed to facilitate escape of supernatant fluid.

FIG. 13 illustrates a fracker packer device for sealing the base of an injection well with a liquid jetting pass-through plumbing to enable the delivery of water to a jet or jets at the base of a well with the slurry pipe and inflatable packer in place.

FIG. 14a depicts a system diagram for the input, outputs, and the preferred embodiment of the methodology of the desired topography computation associated with the predictive topographic modeling subsystem.

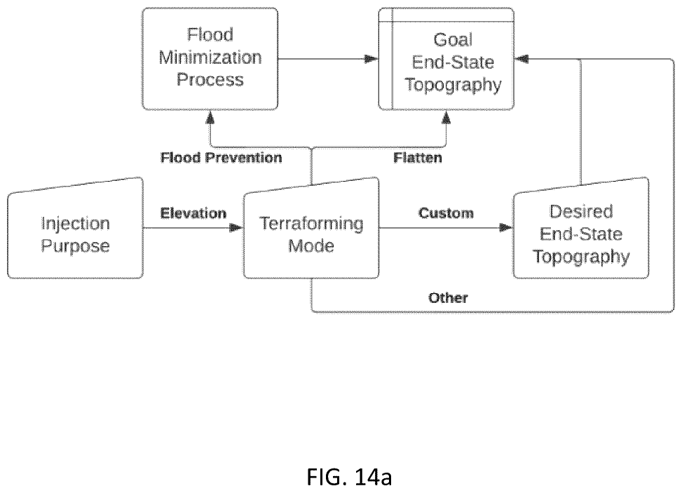

FIG. 14b depicts a system diagram for the input, outputs, and the preferred embodiment of the methodology of the initial geospatial mesh computations associated with the predictive topographic modeling subsystem.

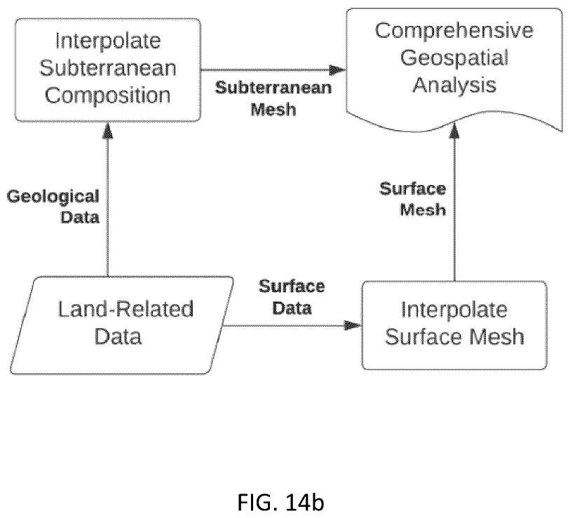

FIG. 14c depicts a system diagram for the input, outputs, and the preferred embodiment of the methodology of the topography modification iteration step associated with the predictive topographic modeling subsystem.

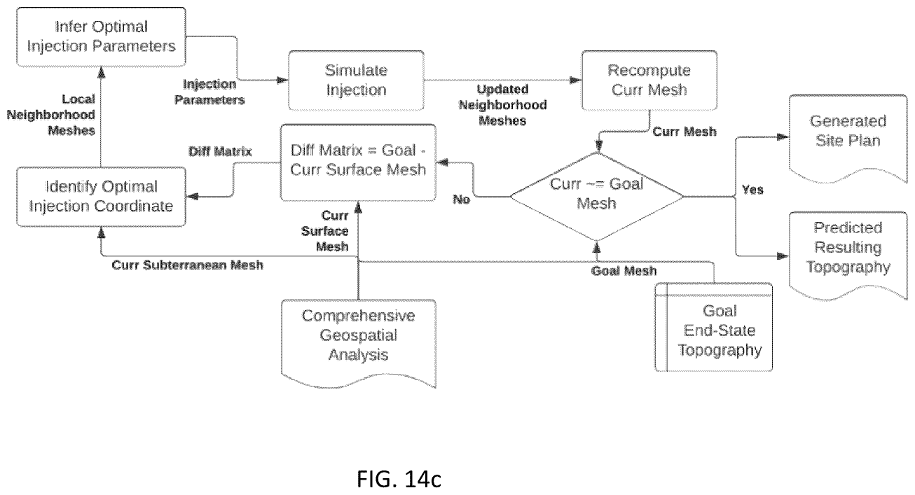

FIG. 14d depicts a system diagram for the input, outputs, and the preferred embodiment of the methodology of the sequestration-specific computations associated with the predictive topographic modeling subsystem.

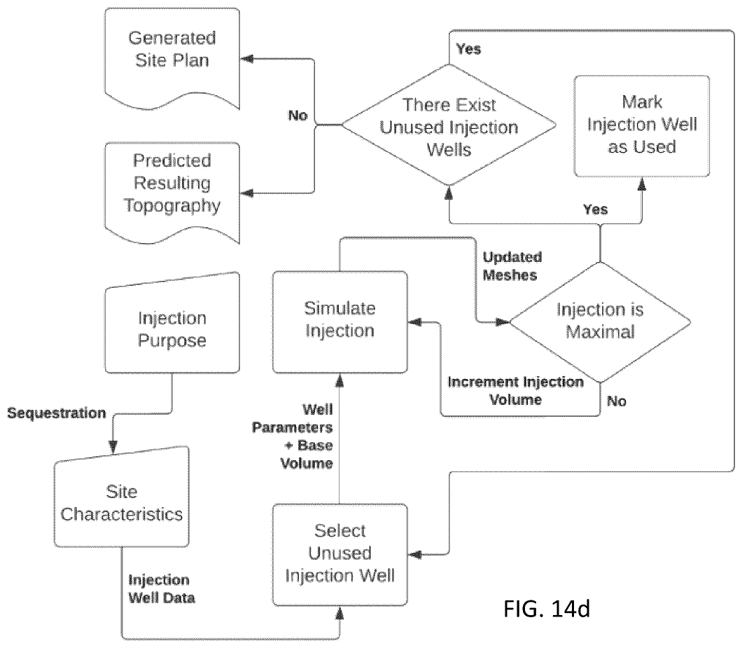

FIG. 15 depicts a system diagram for the input, outputs, and the preferred embodiment of the methodology of the real-time system control subsystem.

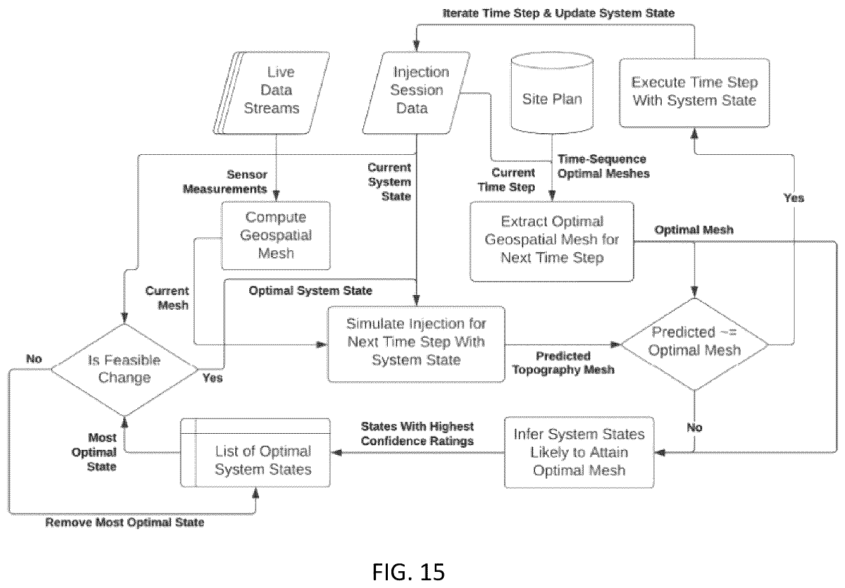

FIG. 16a depicts a system diagram for the continual feedback loop established within the injection parameter model.

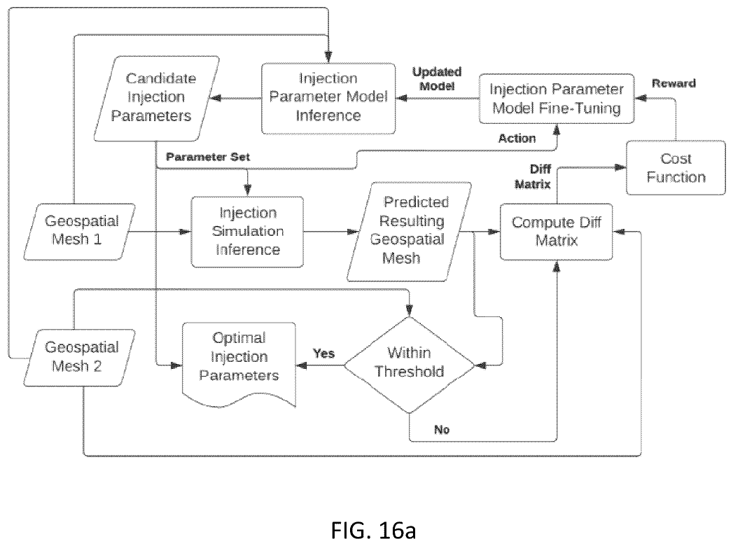

FIG. 16b depicts a system diagram for the continual feedback loop established within the injection simulation model.

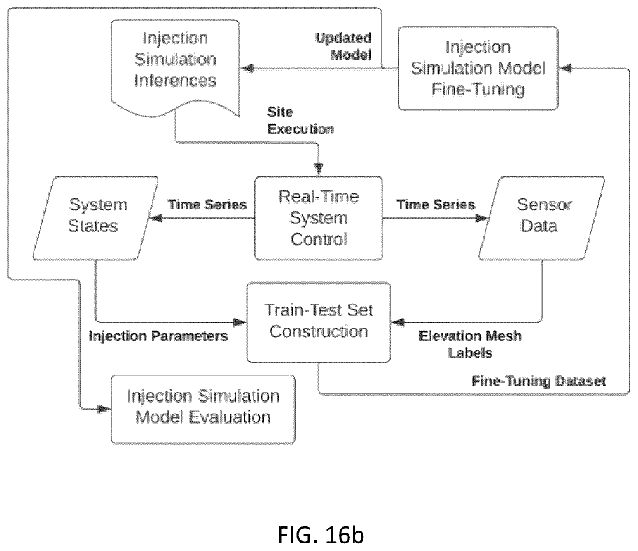

### DETAILED DESCRIPTION OF THE INVENTION

In the following detailed description of exemplary embodiments of the invention, reference is made to the accompanying drawings (where like numbers represent like elements), which form a part hereof, and in which is shown by way of illustration specific exemplary embodiments in which the invention may be practiced. These embodiments are described in sufficient detail to enable those skilled in the art to practice the invention, but other embodiments may be utilized and logical, mechanical, electrical, and other changes may be made without departing from the scope of the present invention. The following detailed description is, therefore, not to be taken in a limiting sense, and the scope of the present invention is defined only by the appended claims.

In the following description, numerous specific details are set forth to provide a thorough understanding of the invention. However, it is understood that the invention may be practiced without these specific details. In other instances, well-known structures and techniques known to one of ordinary skill in the art have not been shown in detail in order not to obscure the invention. Referring to the figures, it is possible to see the various major elements constituting the apparatus and method of the present invention.

### Section I: Techniques and Apparatus

The present disclosure provides techniques and apparatus to enable the protection of terrain and structures from inundation by ground level elevation as well as to protect structures from seismic events by altering the mechanical character of the ground. Terrain may be expanded, and islands may be formed if the process is used in shallow marine areas. Additionally, benefits are accrued by the use of the invention by avoiding hazards due to fires and pollution which would result if the invention were not implemented. These disclosed methods and systems will enable leveling of structures in cases where past differential settling has damaged them. The disclosed systems and methods are excellent ways to achieve long-term sequestration of carbon to reduce atmospheric accumulation of carbon dioxide.

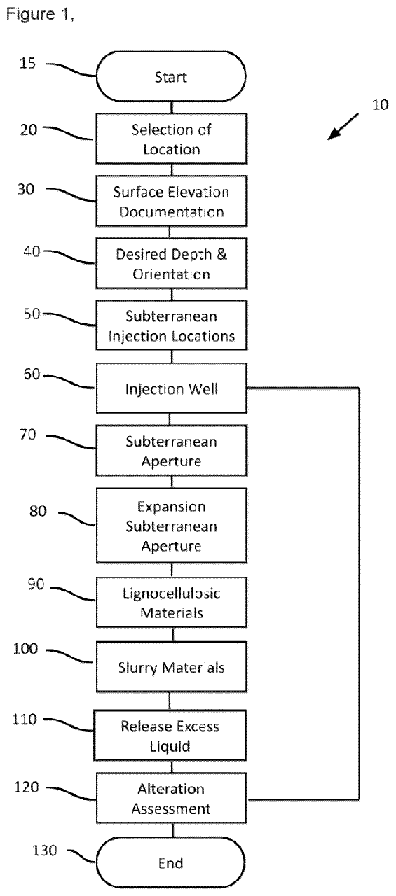

In one arrangement, the present disclosure comprises in one aspect a method 10 of protecting structures with subterranean injection, including a sequence of steps as illustrated in FIG. 1. According to this method 10, the process starts at step 15 and proceeds to a first step 20 wherein selection of a suitable location is done where the advantages of structural or terrain protection from inundation or seismic events or both may be advantageous.

The process then proceeds to step 30 where surface elevation documentation is next accomplished in conjunction with the placement of surface elevation and inclination change sensors.

After step 30, the process proceeds to step 40 where a determination of the desired depth and orientation of prospective subterranean solids is done by evaluation of soil borings or other information about local geotechnical character of the site.

Then, the process proceeds to step 50 where a determination of the number of subterranean injection locations is done which will best accomplish the elevation of ground and contouring of surface or alteration of local soil mechanical properties as desired.

After step 50, the process proceeds to step 60 where a creation of an injection well is done which will enable transfer of solids from the surface to the selected subterranean depth. In one arrangement, this will entail drilling, direct piercing, sonic drilling, or auguring to the appropriate depth. Placement of pipe or tubing from the surface to the bottom follows if not used in the process of creating the well. The well bore may then be sealed to the pipe or tubing so as to ensure that fluids pumped into the injection well cannot simply flow back to the surface or other substrata around the pipe or tubing via the well bore. This sealing may often be accomplished through the use of cementitious sealing plugs, polymer foams, reactive grouts or inflatable plugs that isolate the fluid and pressure at the base of the hole from that of the well bore. Placement of equipment to monitor the subterranean conditions such as a pressure transducer may also be done as needed. Connection of the well to the fluid preparation, pressurization, movement, monitoring and control systems is done.

Next, at step 70, creation of a subterranean aperture is accomplished by injection of fluid under pressure into the subterranean space. This step may involve the use of high-pressure jets to help direct the shape of formation of the aperture or additives to increase the fluid viscosity and reduce aperture leak off of injection fluid.

At step 80, expansion of the subterranean aperture is next accomplished by the injection of fluid under pressure.

At step 90, placement of lignocellulosic materials in the aperture is completed with the injection of an aqueous slurry.

Then, at step 100, rinse of slurry materials from transfer piping with the aqueous solution is done as needed.

And then at step 110, release of excess liquid from the aperture is next allowed which is called relaxation. This may be done by allowing fluid to leak out into adjacent subterranean structures or the fluid may be removed at the surface by release of pressure or by controlled pumping. The aperture surface settles over the included solid fill and can compact the subterranean solid fill. This relaxation allows the included solids to bear the weight of the overburdening earth rather than for the fluid surrounding the solids to bear this weight. With time subterranean fill will also become more thoroughly saturated with fluid increasing the density of individual fill particles and potentially causing them to swell.

And finally, at step 120, assessment is completed of alterations in surface elevation and inclination changes. The process concludes at step 130.

As illustrated, process steps 60-120 may be repeated any number of times to elevate and shape the terrain. The mechanical character of the ground may be altered with successive injections and fluid leak off or relaxation events. This change in mechanical character of the ground may have the effect of altering the orientation of subsequent aperture growth. Larger areas may require that a large number of wells be created and any given well may undergo injection, material distribution, and relaxation cycles multiple times.

The aqueous slurry injected in the lignocellulosic placement at step 90 is created and controlled on a separate apparatus which is further described. In order to illustrate the best mechanism and implementation of this slurry preparation apparatus it is illustrative to describe the objectives and advantages of the use of lignocellulosic material for subterranean slurry injection. Once again, lignocellulosic material is understood to include lignocellulosic materials of all descriptions with or without reactive processing which originated from plants or other photosynthetic organisms as stated earlier.

An object of the present disclosure is to describe systems and methods to reduce the cost for protection of structures and terrain from inundation. The surface topology of terrain may also be altered in a number of ways. This cost reduction derives from a number of different improved aspects of the disclosed systems and methods relative to the use of mineral solids or sediments to elevate terrain and structures as described in Germanovich and Murdoch. These improved aspects include at least the following: Transportation cost of solids to be injected is significantly reduced, Slurry preparation costs are reduced, Slurry injection management and subterranean distribution of solids is simplified, and Certain costs associated with location surface preparation and post-injection clean-up are eliminated.

More details on achieving by the presently disclosed systems and methods of each of these cost reduction advantages and their importance follows.

If it is, for example, desired to elevate a hectare of terrain or structure by 1 meter of altitude, the minimum requisite volume of solids exceeds 1 meter×10,000 m²=10,000 m³. The minimum requisite volume of solids exceeds this number because the edges of the elevated area must be tapered down to meet the old surface of the earth. The taper volume requires additional solids with the quantity dependent on the slope of the taper. For very large areas of elevation the volume of solids required approaches this minimum volume per area due to the diminishing significance of this edge effect. The minimum volume also exceeds this minimum value because a given volume of solids when measured as delivered to the surface location will compact and densify after placement in a subterranean space and exposure to compaction forces such as the mass of the soil overburden the solids will support.

Exposure to water will also have various densifying effects with time. The cost of some solids including wood chips and dredge spoils may often be very low or even less than zero. This would mean that the solid was removed from one location for a different purpose than direct sale as is often the case with solids removed to deepen a navigational channel or trees removed for landscaping or fire suppression purposes. The delivered cost associated with a new location where the solids are desired is largely determined by the cost of transporting the solids from the site of removal to the locations where the solids are desired. This cost is most frequently defined for bulk materials by density.

The bulk density of chipped lignocellulosic plant materials is variable but frequently in the range of 0.15 to 0.35 g/cc while mineral solids such as sand and sediment are frequently in the bulk density range of 1.5 to 2.0 g/cc. The bulk density includes the open space between particles and perhaps the water that may fill them and so is lower than the particulate or intrinsic density.

A truck or other transportation device is usually allowed a certain maximum mass to transport and thus the volume transportable at this maximum mass may be expected to be inversely proportional to the densities of the materials. It is expected that the delivered price of lignocellulosic materials will range from one tenth to one quarter the cost of sand, soil or sediment because a comparable volume of the mineral materials would require four to ten times as many truck trips to transport.

By converting the bulk densities of lignocellulosic material and also sediment to volumetric ranges delivered per truck shipment the lower transportation cost advantage of lignocellulosic material becomes apparent.

For a truck able to carry 20,000 kg, 57-133 m3 of lignocellulosic material versus 10-13 m3 of mineral materials may be brought with each truck. To elevate each hectare of terrain and structures by 1 meter exclusive of edge taper effects and compaction consideration would therefore require perhaps 75-175 truckloads of wood chips as opposed to perhaps 800-1000 truckloads of sediment, sand or other mineral material.

Information from The California Department of Transportation is useful in order to gain perspective on the importance of reducing costs associated with mineral fill required to protect structures from inundation. Caltrans estimates that 3,000 lane miles in California will ultimately require protection and assuming 10 m total lane width at 1 m elevation and $40/m3 for purchase, preparation and delivery of fill a cost of nearly $2 billion would be incurred and require nearly 4 million truck trips. For comparison, total funding for the State Transportation and Improvement Program is about $3 billion annually. Fill cost would be only a fraction of project cost and highways are a small fraction of terrain and structures that require protection in California. Sea level rise has been called the largest engineering problem mankind will face.

A low-cost mineral fill material, for example the dredge spoils and sediment referenced by Germanovich and Murdoch would contain unwanted coarse and problematic materials, for example large rocks, metal cans, rope and branches as may have accumulated at the bottom of navigational channels or elsewhere. When dredge spoils are pumped, very large centrifugal slurry pumps are needed to pass the majority of these large contaminants. The large foreign materials would require removal before subterranean injection. This removal could be done in either a dilute aqueous vibratory screening operation or in stagewise cyclonic or gravity settling equipment.

In each of these cases, a large quantity of contaminated water would be produced which would require a large settling pond. This type of operation is problematic in many areas because dredge spoils often contain hazardous chemicals such as heavy metals and often must be tested for such. Once processed in a dilute slurry, in order to remove the bulk of the spoils, it is likely that a slurry of very fine and therefore very slow to settle clay-like particles would be produced. This clay slurry would likely contain still a higher level of these hazardous contaminants. This potentially hazardous clay slurry would likely require significant processing to render suitable for disposal.

An alternative would be to allow all the dredge spoils to dry and then process them with a regrinding and dry screening operation. This drying would require a great deal of time and land area. Either wet or dry processing of dredge spoils to remove oversize materials is necessary to create a manageable material for concentrated injection into narrow subterranean apertures. This additional processing may add substantially to the $30/m3 cost figure referenced earlier which applied only to surface dumping of spoils.

Surface sourced mineral materials for injection would also require screening to eliminate oversized inclusions. Only surface sourced materials that could form thick pumpable mud would be suitable for subterranean injection. Some soil and sediment contain some quantity of partially decomposed organic matter but this is an insignificant fraction in many cases.

Lignocellulosic fill materials are an extremely attractive alternative to mineral fill. A significant advantage of the subterranean injection of lignocellulosic material as described herein is that they have high porosity and lower density while in some cases retaining high mechanical strengths. The porosity enables these alternative solids to form slurries that do not settle as rapidly as mineral solids of comparable dimension. Sand and other dense mineral materials often have particulate (intrinsic) solid density of about 2.7 g/cc and thus settle readily in water at a rate determined by their particle size and the viscosity of the water within which they are suspended. Biological origin solids may float in water, be neutrally buoyant or sink based on porosity of their structure and the degree of water saturation of these air-filled pores.

Most wood materials and similarly porous biological materials may have a pressure dependent buoyancy in fluid. Increased pressure will progressively collapse included air space and shift these materials toward higher apparent densities as they approach their molecular density. The molecular density eliminates porosity effects. The molecular density of lignocellulosic materials is approximately 1.45-1.55 g/cc dependent on the ratio of lignin to holocellulose. Thus, they may sink or float in an aqueous media depending on wetting and the volume of the included vapor space.

Lignocellulosic material may alternatively be made to sink, or float based on pressure, duration of exposure to the liquid, and agitation. Lignocellulosic materials that have undergone reactive processing vary in their densities and porosity depending on the conditions of the reactive process.

Lignocellulosic materials inevitably contain some fraction of sand, soil and other mineral material as incidental contamination. Some lignocellulosic materials such as algae gathered in coastal areas will often contain contaminants such as plastic and other foreign materials. In many cases, these inclusions do not change their fundamental character and suitability for subterranean injection. In fact, the inclusions may make it desirable to use the contaminated materials for subterranean injection rather than other potential uses such as surface soil enhancement.

Improved slurry management tools can be important when seeking to inject slurries with larger particle sizes. Fine particles sizes, for example, 20 micrometer diameters, are necessary with particles that have intrinsic densities of 2.6-2.8 g/cc or higher and thus have a negative buoyant force proportional to particle density−fluid density (=1.7 g/cc in the case of sand and water) in water. Biologically sourced materials have buoyant forces in water that may be either positive or negative and generally of a magnitude less than 25% as large as most mineral materials of comparable size. Often these buoyant forces are instead about +/−0.2 g/cc or less depending on the relative quantity of included gas in the plant cell structure. This low buoyancy or sinking force enables slurries to be stable with particles that are dramatically larger.

It is important to note that there will be near-neutrally buoyant particles as long as the pressure is below a critical high mark which would result in all wood or plant chips below a finite size sinking. In an effort to understand this phenomenon a pressure of 827.4 kPa was applied to a slurry of fir bark fines screened to pass a #4 mesh square hole screen. 827.4 kPa resulted in 95% of chips sinking but was insufficient to render approximately 5% of chips negatively buoyant and they remained floating in the water at 25° C. 827.4 kPa (120 psi) is approximately the pressure that would be encountered at a depth of 120 ft below ground surface near the sea. Larger wood/plant chips might have sealed air cavities that do not fill with fluid immediately but may ultimately saturate if included gas can dissolve in fluid or if fluid can displace the gas toward the space between individual chips.

A slurry of lignocellulosic materials or biologically sourced material (collectively called lignocellulosic material but understood to also include 0-100% leaves, grass, or any other plant material, also called “biomass”) performs very differently in a water slurry in comparison to mineral slurries that commonly have intrinsic particle densities of approximately 2.7 g/cc. Wood chip slurries do not consolidate and solidify after settling in the way that mineral or rock/soil materials are observed to do. As an example, a mixture of minus 60 mesh sand with 20% clay soil from Marin County California after settling in a 200 ml glass jar cannot be completely resuspended with vigorous shaking unless the jar is inverted numerous times. The solid mixture settles with larger particles at the bottom and progressively finer material toward the upper portion of the settled mass.

In another trial a slurry of fine particles (8-20 micron) magnetite is observed to form a solid-like plate and cannot be resuspended without recrushing and intense shearing. This behavior may be characterized as partial cementation. Still another example of a mineral slurry is a minus 40 mesh clinoptilolite zeolite which also compacts after settling and partially cements. Hard physical scraping and agitation is enough to partially resuspend this material.

Without the addition of thickening clay fines to counteract this settling, additives which increase the viscosity must often be used. Common additives for hydraulic fracturing slurries used to deliver mineral proppants into petroleum well geological structures include polyacrylamide and polysaccharides such as guar gum. The particle size of the proppants must be small and the viscosity of the fluid sufficient to enable transport of the proppant horizontally into the fracture without proppant settling or screening out.

When lignocellulosic material is submerged in water or brine it saturates over a period of time with water, the rate of saturation with water is initially more rapid but slows after a number of hours and near-complete saturation may take years. Even after years in a fully saturated earth environment, some portion of the gas contained in the interstices of plant structure may persist. The mobility of the water phase surrounding the chips is expected to control the rate of removal of residual gas from the wood. If the water phase is immobile, it may be that the original air is retained. Additives such as guar gum, xanthan gum or fine particle size clay minerals that increase the fluid viscosity may reduce the rate that gases can migrate through the fluid by reducing convective currents and by immobilizing gas bubbles so that they may not freely move in the fluid.

When lignocellulosic materials are injected beneath the soil surface, an important eventual consideration is decomposition. Reduction or cessation of decomposition may often be desired. Maintenance of an oxygen-free or anoxic environment is crucial to avoid aerobic microbial decomposition. Depth beneath the soil surface is an important consideration to ensure an anaerobic or anoxic space for wood placement. In many areas with clay soils a meter below ground surface is more than adequate to reach a permanently anaerobic region. In more porous sand or loam soils air penetrates farther. or Exclusion of fixed nitrogen in the form of ammonium ion, amines, nitrates, high nitrogen content plant material and other available forms for microbes is important in pursuit of reduced decomposition. Exclusion of phosphorus is additionally important.

It is also possible to affect decomposition by manipulating the pH of the wood chip environment or by adding inhibitors or biocides. Another mode to reduce decomposition would be to enrich the environment with the products of decomposition whether those products are organic acids, CO₂, methane or other constituents. Some decomposition of wood chips is inevitable, and this may result in the presence of vapor bubbles in the subterranean space from accumulation of CO₂ and methane to accompany any residual nitrogen or other constituents of residual air. If decomposition reaction products are retained in the wood environment and not allowed to exit, the degradation rate must ultimately decline. Saturation with reaction products such as hydrogen sulfide gas in the case of near-anaerobic decomposition by sulfur utilizing microorganisms can ultimately stop degradation and poison microorganisms responsible for decay.

An example of reaction rate decline due to the buildup of reaction products is fermentation of sugar-containing fluids by yeast. Elevation of alcohol content in wine or beer will ultimately stop further biological decomposition of sugars to alcohol. Limiting availability of necessary reactants or nutrients and buildup of reaction products will both limit decomposition of lignocellulosic materials in a subterranean environment.

The solubility of gases such as oxygen, carbon dioxide, and methane in the aqueous fluid surrounding submerged lignocellulosic particles is an important determinant of decomposition reaction rate. The quantity of reducible reactants such as oxygen for aerobic decomposition or sulfate, iron, manganese and nitrate ion for partially anoxic decomposition determines whether the whole lignocellulosic material can be decomposed and to a large extent how rapidly that decomposition will occur.

In a fully anoxic environment, the lignin component of lignocellulosic materials does not degrade and the rate of decomposition of holocellulose which is the combination of the carbohydrates cellulose and hemicellulose that makes up cell walls in plant material is greatly reduced. Anoxic decomposition of carbohydrates involves methanogens consuming low molecular weight acidic molecules that are produced by other microbes. Anoxic decomposition produces a mixture of carbon dioxide and methane gas. If the reaction products are allowed to accumulate the reaction may be slowed or stopped, as mentioned earlier. In a subterranean environment the condition is effectively always anoxic below the water table or more than a meter underground if dense soils are present.

Coastal or riparian areas subject to inundation are often anaerobic due to close proximity to the subterranean water table. Lignocellulosic materials pumped into a subterranean space of adequate depth or below the local water table are generally only subject to anaerobic decay assisted by methanogenic microbes once initial oxygen available in pore spaces are consumed. This anaerobic decay can only proceed to the extent that reaction products (wastes) can exit the subterranean space. Carbon dioxide and methane can migrate as gases through subterranean spaces. Gases such as oxygen, methane and carbon dioxide are significantly less soluble in water when sodium chloride and other salts are present. Because of this brackish water like sea water can slow delivery of reactants and removal of wastes for microbes decomposing lignocellulosic materials and thus increase the longevity of these materials in a subterranean space.

The determination of a minimum depth of injection to ensure that wood chip materials will persist in the subterranean environment guides depth selection. The wood chips must be injected below the permanent anaerobic surface level or horizon of the soil. The elevation below ground surface of the transition to anaerobic and anoxic conditions will be different for each soil type and geographic region. The anaerobic depth varies with local water table depth, soil compaction and soil type.

The anaerobic depth will be the lessor of: 1) The local water table as determined by soil cores or one skilled in local hydrology, 2) 1 meter below an area of soil with 20% or less void space as determined by soil cores, 3) The depth at which redox testing of soil chemistry performed using direct measurement by one skilled in the art shows a reducing condition, and 4) 5 m deep if the soil is fine grained such as silt or clay.

As further explanation soil with 20% void space or less is too compacted to allow air passage and so can stop air penetration to zones below. The presence of iron as Fe(II) as opposed to Fe(III) indicates a reducing soil environment and can signal to one skilled in the art that soil at and below that depth will be anaerobic. Direct measurement of the redox potential of the soil to indicate a reducing environment is an alternative method to signal an anoxic state because of anaerobic conditions or consumption of nearly all available oxygen by soil components.

This anaerobic depth may be considered a minimum distance below ground level needed to avoid wood chip decomposition by aerobic microorganisms but soil stability for structures may require injection still deeper as further defined below. The desired level of wood chip compaction by the overburdening soil will establish a deeper minimum depth for material placement if structures are to be supported by the injected lignocellulosic material. The indicated injection depth would therefore need to be below the anaerobic transition horizon and also the minimum depth to achieve adequate compaction. The physical properties of fibric peat soil including its friction angle and shear strength increase with increasing consolidation pressure. This is true with other varieties of organic fibrous materials.

At a depth of 5 m the consolidation pressure would be about 100 kPa and the shear strength and friction angle of a compressible wood chip layer would often be on a par with or in excess of the shear strength and friction angle of clay or silt soils. At this or greater depth clay soil types would be reinforced by a layer of wood chips. Biomass materials come in a very broad variety of characteristics and selection of a fibrous material with particle size in the range of 2 mm to 25 mm would best serve this reinforcement character. Deeper injection than the minimum depth determined above is economically advantageous if fewer well bores are desired and a greater injection quantity per well is sought. Geotechnical engineers must be consulted to determine depth required beneath any structure with more than two stories. An injection depth of 5 m is a practical minimum and 100 meters is considered a practical maximum depth of injection.

Saturated wood chips are highly porous and subject to significant compaction as the depth of the overburden increases and thus normal stress state of the woodchip soil increases. Fully saturated wood chips will undergo some additional compaction due to creep and thus the level of porosity and hydraulic conductivity will decline over time until a steady state is reached. The level of creep consolidation will increase with increasing depth due to higher loads from the overburdening soils.

Lignocellulosic material when placed in water often has sequestered vapor (mostly air) held inside residual plant structure that can gradually escape. The process of this gas escaping from plant tissue may be through physical replacement by water. This pushes vapor bubbles out. Another form of escape is through dissolution in the fluid. Oxygen is approximately twice as soluble in water as is nitrogen and air contains only approximately 21% oxygen gas but 78% nitrogen. Available oxygen will be consumed by aerobic lignocellulosic decay organisms. It is thus expected that the vapor bubbles inside the wood structure will more rapidly be depleted of oxygen than they are depleted of nitrogen.

Initially fresh lignocellulosic materials are quite easy to suspend in water or brackish water as they become wet and take up moisture. Smaller sized chips tend to saturate with water faster and often sink within a short period. Lignocellulosic slurries are dramatically simpler to resuspend after settling in comparison to mineral slurries. A portion of the slurry resuspends instantly with fluid movement because its buoyancy is nearly neutral.

Larger lignocellulosic particles will resist decomposition longer and thus it is desirable to pump larger Lignocellulosic particles into subterranean spaces. Green waste and wood chipping operations create a range of particle sizes. Uniformly fine lignocellulosic materials such as sawdust of perhaps 2 mm length by 1 mm width can be quite easy to suspend in an aqueous slurry. Sawdust requires more energy to produce and thus once limited supplies of “waste” sawdust materials are exhausted, sawdust would be a much more expensive form of lignocellulosic materials for slurries than coarse chips. Sawdust sized material also has a lower bulk density than coarser materials which in turn means a given mass of sawdust will require more water to slurry than a comparable mass of coarser lignocellulosic material. Sawdust is also more compressible than more coarsely sized lignocellulosic materials such as bulk wood chips produced by tree trimming services.

A 100 mm thick aperture filled with a sawdust slurry might thus need to lose much water during the relaxation stage mentioned in the sequence of steps for injection to place materials in a subterranean aperture. This relaxation step allows the injected solids to begin to support the full weight of the overburdening earth. The quantity of water lost from the aperture during relaxation by a wood chip slurry with average particle size of 20 mm might be half that lost in a similar relaxation conducted on a sawdust slurry of average particle size 2 mm if both slurries were formed with a similar dry volume of sawdust and woodchips.

The ability to slurry and inject large particles of lignocellulosic materials has the advantages of significantly lower size-reduction costs, slower degradation in a subterranean environment, lower water required to form slurries, and consequently lower water loss requirement during relaxation of the filled subterranean aperture space. The near-neutral buoyancy of lignocellulosic materials is advantageous in this regard. It is expected that particles up to or indeed in excess of 25 mm in any dimension may be pumpable with appropriate pump systems such as progressive cavity or piston pumps that include large check valves. Well piping diameter must be at least four times the diameter of the largest particles.

Subterranean apertures may be of any orientation, vertical, inclined, horizontal or any complex intermediate shape. Horizontal apertures when filled serve most effectively to elevate the surface of the ground. Slurry flow in a horizontal space poses important challenges. As the fluid flows in a horizontal direction, solids denser than the fluid (which usually has a density close to that of water) will sink until they reach the floor of the aperture. To reduce the rate at which solids settle the viscosity of the fluid may be increased or the size of the solids may be reduced. As the viscosity of the suspending fluid increases, the requisite difference in pressure between the starting point of the fluid and its ultimate endpoint along a horizontal plane increases. Pumping more viscous fluids requires more energy than less viscous fluids over a similar path. A more viscous slurry will prevent solids from settling and also from excessive contact friction with edges of solids such as encountered in sharp pipe bends or tight underground spaces.

When solids contact edges in an inadequately viscous fluid they are more easily stopped and can “screen out” or form a packed bed at the edge or transitional space. Additives such as guar or xanthan gum or mixtures of the two as well as fine clay materials like sodium bentonite clay can increase the viscosity of fluid and help avoid screen outs or immobilization of solids at tight transitions or bends.

A mineral solid slurry must be maintained at an adequate agitation velocity or the solids will settle unless the solids content is high enough to result in a thick paste or mud. There are important problems caused by either a thin and easily pumpable mineral slurry or a thick and slow settling slurry both above and below ground. The thin slurry will fill the subterranean aperture with a large volume of water which will still contain fine clay particles and be quite dirty in appearance and potentially able to pollute surface water. Though the thin slurry is quite pumpable it will not carry adequate solids to prop up the terrain and the extra water will require a long period to escape the aperture. Relaxing the aperture so that the solids are bearing the weight of the overburden may take a long time. Reuse of a given aperture space may be challenging because the fine clay particle may clog the pores of the space around the solids as the additional water attempts to exit during relaxation. Therefore, relaxation may take progressively longer and eventually the aperture may not function for additional injections.

The thick mud will result in a high pressure differential between the injection point and the peripheral extent of the aperture. This pressure differential results from the Bingham plastic rheological nature of the mud and may distort the shape of the aperture. The distorted shape may result in the central or material entry portion of the aperture filling with a disproportionate quantity of the solids while the periphery has much less material. A thick mud is also potentially a major contamination issue for the surface area around the well in the inevitable event of a spill.

Lignocellulosic slurries by comparison are quite easy to sort, manage and use in a subterranean injection operation. They may be preselected to include only particles of a certain size range with trommels or vibratory screeners without need for drying or fines management systems. Lignocellulosic materials are not generally considered problematic or contamination when spills occur on the surface. They may often be removed with rakes, brooms, leaf blowers, or vacuums. They may also be intentionally placed on the surface to act as a weed controlling mulch or landscaping material. When structures are elevated, the surface placement of residual lignocellulosic mulches, for example wood and bark mixes, creates a particularly beneficial habitat for methanotrophic bacteria. Therefore, the placement of mulch on the surface of an elevated area may be considered an important part of the process of ensuring that little or no methane escapes to the atmosphere. It is during the first few years after placement of the subterranean fill when whatever anaerobic evolution of methane from the subterranean space is highest. Methane produced by anaerobic organisms is generally understood to peak shortly after placement and decay thereafter in the ensuing few years.

The water used to produce a lignocellulosic slurry does not generally become contaminated as water that is used to make a mud or mineral slurry is observed to become filled with fine muddy clay particles. If freshwater is used for slurry formation, there is generally no contamination issue on the surface in the case of slurry water spills and therefore no reason to expend effort avoiding surface water spills. Mineral slurry systems would require surface protection systems to trap the water and recover and potentially haul it away after use. This activity adds substantially to the cost of using the mineral slurry for terrain elevation. No such cost is associated with the use of a lignocellulosic slurry except perhaps in unusual cases.

It is expected that local river water, seawater or brackish water will be used for slurry production in many areas when terrain is elevated immediately adjacent to such waterways. If this can be done it can significantly reduce costs when treated freshwater is more valuable. The fact that lignocellulosic slurries do not add significant contamination to waterways enables this procedure.

As noted previously, FIG. 1 illustrates a method 10 of Protecting Structures with Subterranean Injection of Lignocellulosic Solids.

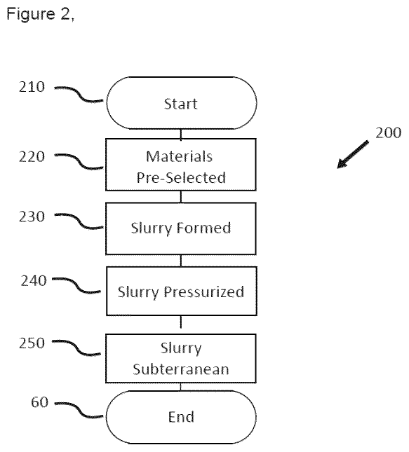

Placement of a subterranean slurry as described in step 90 of the general procedure steps enumerated earlier may now be more fully described in a detailed sequence of steps. For example, returning to FIG. 1 and the process 10 illustrated therein, at step 90, placement of lignocellulosic materials in the aperture is completed with the injection of an aqueous slurry. FIG. 2 illustrates a method for preparing lignocellulosic materials. As illustrated, the process starts at step 200 and proceeds to step 210, where the materials are preselected to include the variety or varieties of lignocellulosic materials that are most desirable and those other constituents of the slurry to be formed subsequently.

Then, at step 220, the slurry is formed, an optional post-selection process may be utilized and the slurry is brought to the desired solids level.

Then at step 230, the slurry is pressurized.

Then, at step 250, the slurry components are methodically placed in a subterranean space using a sequence of steps which best enables construction of the subterranean solids mass that is most suitable.

Each of these steps will be described further to add detail and understanding. For example, the pre-selection process at step 220 is guided by knowledge of how different materials contribute to the slurry formation and subsequent solids placement using the slurry. Lignocellulosic materials may be selected based on species of plant material, size or shape of plant material, porosity of plant material, degree of water saturation, or degree of decomposition. Additional slurry components such as finely divided mineral solids, chemicals, binding agents and viscosity adjustment agents such as guar gum, cross-linkers and breakers, which are used in hydraulic fracturing, may also be beneficial in the slurry or may be desired in the ultimate solid mass to be placed in a subterranean location.

An important selection criterion is fiber length. Peat soils are notoriously poor at supporting structures. Peat is a decomposed form of lignocellulosic material, Fibric peat soils are less decomposed and contain fibers that serve to enhance their shear strength. The normal force applied to the sample compacts the fibric peat and its shear strength as measured by the direct shear test also rises. At or near the surface where there is little compaction pressure the shear strength of peat soils is very low and so these soils are problematic for structure foundations. However, at depths of 5 meters the compaction pressure arising from support of the overburdening soil would make the soil shear strength adequate for fibric peats in some circumstances. Less aged lignocellulosic materials would be expected to follow a similar pattern. More fibrous materials may be desirable at shallow depths and less fibrous materials may be selected for deeper injections.

The aspect ratio or length to width ratio for fibrous lignocellulosic materials significantly affects their strength. Short fibers do not impart as much strength as do long fibers in wood fiberboard products. The strength of wood also varies dramatically with and against the grain of the wood.

The portion of the holocellulose component of lignocellulose may eventually decompose in an anaerobic environment but lignin is generally persistent. Most lignocellulosic biomass will remain even after many thousands of years. The ratio of lignin to holocellulose varies by type of lignocellulosic material as it does with algae and phytoplankton. Most algae for example have little to no lignin and some may have no cellulose.

In certain situations where terrain or a structure is to be elevated, it may be desirable to reduce the potential settling due to decomposition or the possible evolution of methane and carbon dioxide from anaerobic decomposition. In these cases, high lignin species may be desirable or even very high lignin components of a given species. Pine tree bark has nearly double the lignin content of pine wood in many cases. Coconut husks and many nut shells have very high lignin content and may represent both the minimum of decomposition rate and minimum total degree of decomposition among readily available plants or algae. Some species have preservative oils and extractives that discourage decomposition. Redwood and eucalyptus species for example have low decay rates due to protection afforded by other resinous chemical constituents of the lignocellulosic material.

The slow, partial anaerobic decomposition of lignocellulosics will produce methane and carbon dioxide. Most soils contain plant roots that decompose anaerobically and the methane produced feeds methanotrophic organisms in the upper, more oxygenated layer of the soil. Most of the soil produced methane from subterranean plant decomposition does not enter the atmosphere but is instead consumed by methanotrophic microbes before this occurs. Underneath some structures there would be little to no methanotrophic activity and so lower methane emissions are desirable underneath structures in comparison to under adjacent open or plant covered terrain.

For this reason, it may be desirable to use more lignin-rich lignocellulosic materials directly underneath structures to protect these structures whereas more cellulose-rich materials may be quite satisfactory under grassy areas or areas covered by lignocellulosic mulches where methanotrophic activity is enhanced. By the same token more decomposition resistant species, for example redwood tree chips, may also satisfy the desire to reduce methane production under structures.

Larger lignocellulosic particles when injected into a subterranean space do not deform as easily under load as smaller particles. Fifteen (15) mm wood chips will maintain a greater flow rate of water around them than a comparable mass of saw dust under a comparable compression supplied by the weight of overburdening soil. This is significant when the water pressure is removed and the aperture allowed to relax. Fine solids will compress more and coarse solids will compress less which leaves more open water channels. The excess slurry water will exit the injected solids rapidly if those solids are more coarse and slowly if the solids are very fine. A slurry of fine materials may be injected into a space filled with coarser particles to fill the open spaces between larger particles. This creates several valuable opportunities to manage how solids are added to a space over time and over multiple injection events.

It may be beneficial to fill a loose mass of coarse particles added over multiple injection events with a final injection of fine materials to help solidify the solids in place and increase their density by filling gaps between the solids. It is also important as a tool to control where within a subterranean mass of solids water can flow easily and where its flow will be restricted by sawdust filled gaps between larger particles.

The slurry formation process is a key aspect of the art which this disclosure enables. There is advanced slurry formation technology that is well-known to those with skill in the art which enable time-dependent control of slurry viscosity to enable low viscosity at the surface, that rises to higher viscosity when viscosifying agents are crosslinked to thicken the slurry and break open formations and entrain heavy solid proppant particles to drag them into subterranean formations. Chemical “breakers” then chop up the polysaccharides and other long chain molecules that once thickened the slurry to bring the viscosity back down near that of water. The low viscosity liquid can be drawn back out of the formation leaving the proppants behind. The timing of the process is carefully controlled by still other chemicals known variously as delay agents, stabilizers, and activators. The chemical systems and technology developed for the sophisticated petroleum hydraulic fracturing industry would prove very useful in many applications envisioned in this disclosure; however, viscosity control using these systems is unavoidably expensive.

The presently disclosed systems and methods exploit the unique capabilities of lignocellulosic material subterranean injection to protect structures can be most efficacious when used at the very lowest cost because many billions of cubic meters of injection solids must be placed to protect many millions of structures. Doubtless lignocellulosic materials will be used with chemical viscosity control as important embodiments but it is the enablement of the simplest and lowest cost slurries which in the end will be a primary contribution of the presently disclosed systems and methods.

The slurry formation apparatus blends the lignocellulosic materials which may have minor contaminants as mentioned herein with the water which may optionally be brackish and any desired additives such as viscosity control agents or others mentioned earlier to form a slurry with a controlled solids content for presentation to a pump.

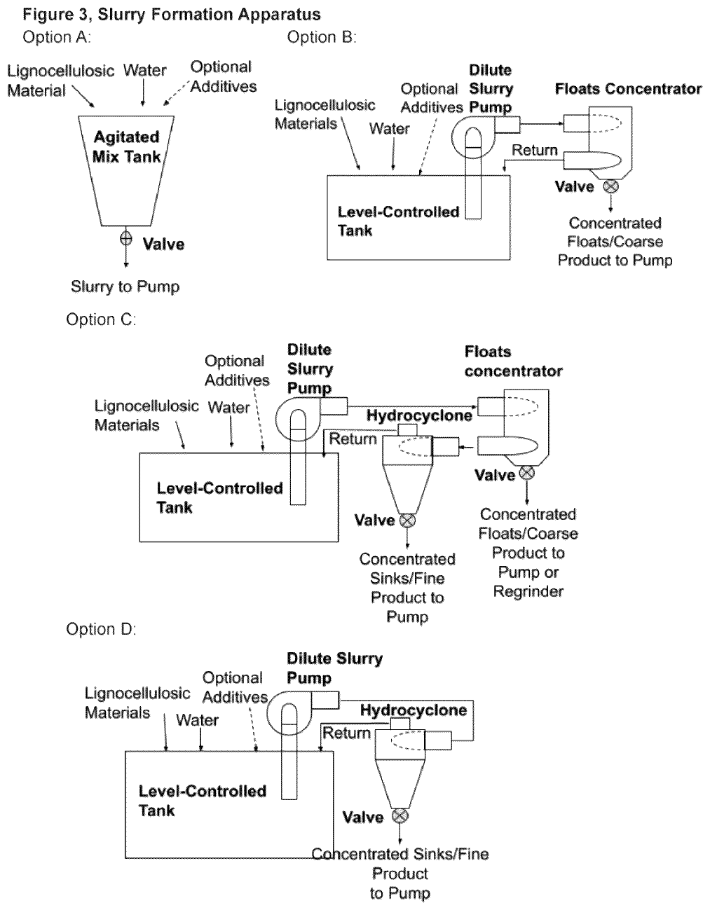

Three varieties of slurry formation apparatus are illustrated in FIG. 3. Option A is a batch system in which fixed amounts or solids and the liquid are blended with an agitator, ribbon mixer or by tumbling as in a batch cement mixer. The batch is pumped into the aperture, the valve is closed and a new batch is prepared.

Slurry formation Option B illustrates the use of a novel device called a centrifugal concentrator for floats. The concentrator allows delivery to the injection pump of lignocellulosic materials that float in water (at surface pressure conditions) and enables control over the slurry concentration. The concentrator creates a spinning mass of wet lignocellulosic material that remains in place above the pump suction. The raw solids and any additives are delivered to a fluid level-controlled tank. A self-priming slurry pump as illustrated in the figure or (other pump variety) then delivers wetted materials that are entrained in the water to the concentrator positioned above the injection pump suction and flowing through a preferably pneumatic pinch valve. If the valve above the injection pump is closed the solids rotating in the mass will build up and be re-entrained by the lower tangentially exiting flow returning to the level-controlled tank. This provides an automatic way to continuously feed the pump a controlled and high concentration of lignocellulosic floats.

Slurry formation Option C includes all the equipment of Option B with the addition of a hydrocyclone on the tangential return line from the floats concentrator. This hydrocyclone removes dense solids that sink in the aqueous fluid. The sink materials will typically contain small diameter particles and particles with high aspect ratio. Sand, gravel and coarse heavies will also deliver at this location. Smaller diameter lignocellulosic materials will typically saturate with water more quickly and their density will rise. These sinking particles may be delivered to a second pump, for example a progressive cavity pump. Both a floats and a sinks stream can be delivered simultaneously to different well locations in dual product mode. These locations may optionally feed different apertures or may feed an expanded aperture in different locations as explained later.

Optionally the dual outlet configuration can be used in conjunction with a grinding circuit. In this mode the floats product (coarse) that delivers to the apex of the first centrifugal concentrator can be dewatered and returned to a grinder for further size reduction. This works well with very fibrous materials that can be problematic to screen in a dry state due to binding and the possibility of fires or dust explosion. The denser and more fine sinks product may be delivered in a concentrated state to a slurry pump for injection. This creates a safer and less energy intensive way to produce fine particles for a slurry. It reduces overgrinding and dust generation as well as energy use while still creating a reliable fine particle concentrated slurry at the hydrocyclone discharge.

The cone angle of the hydrocyclone may be made larger, for example from an industry standard 20 degree included angle to a higher included angle such as 30-90 degrees. The larger this angle the larger will become a rotating bed of dense material awaiting discharge from the apex. A level sensor in the small feed cone above the dense discharge pump may be used as a control signal to adjust the diameter of the pneumatic apex. A low pump feed level would result in a signal to increase the apex diameter by reducing the air pressure in the pneumatic apex of the hydrocyclone. As more material is withdrawn from this rotating mass by increasing the dense pump outlet volume and therefore increasing the controlled apex diameter, a higher fraction of the incoming feed material will take a place in the rotating body of solids awaiting discharge. As the discharge volume decreases, more rotating material in the bed will instead be re-entrained by the flow of fluid exiting the vortex of the hydrocyclone with residual float solids in the feed and will report back to the initial slurry tank.

A unique characteristic of this slurry formation option operating in dual product mode is that the relative amount of production of both the floating (coarse) material and the denser sinking material will vary to a significant extent with the relative rate of their withdrawal by their respective pumps. Therefore, if more dense material is required the dense removal pump rate may be increased and this will have the effect of raising the percentage of the feed that reports to dense material because the size of the rotating bed of material is smaller and more solids will join the bed at the margin of material close to the sink/float cut point of the feed. Adjusting the apex or vortex diameter in the floats selection concentrator and the cone angle, vortex finder diameter, and apex diameter of the dense selection hydrocyclone enables controlled partition of many varieties of lignocellulosic feeds. Each variety of Lignocellulosic material may be partitioned into a more buoyant light (and often coarse) fraction and a more dense heavy (and often fine) fraction over a wide range of ratios of floats/heavy flow splits.

Alternatively the floats concentrator can be bypassed and the dilute slurry pump made to feed only the hydrocyclone as shown in slurry formation apparatus option D. If the hydrocyclone is used as in this option a sinking particle stream alone is available and any floats will be returned to the dilute slurry tank. This optional configuration is useful when only fully saturated fine products that sink are desired in the slurry placement and this mode is a single product mode.

The level of concentration of the slurry depends on the variety of the pump to be used in addition to the flow rate required to open the subterranean aperture and the requisite pressure. A centrifugal slurry pump is an attractive option if injection pressures measured at the surface are 500 kpa or lower. Centrifugal pumps will have significant pressure limitations when high pressures are required to create fractures but the pressure required to fill an open aperture is often significantly lower than that required to create a fracture. Centrifugal pumps work well in situations where the formation aperture to be filled with solids is quite porous and at a depth shallower than 23 m. Centrifugal pumps work better on lower solid volume fraction and so more water per given volume of solids placed in the aperture must escape the structure to allow the solids to carry the overburden.

A head box can also be used in unusual situations where a 20-50 m high tower or hillside is immediately adjacent to the injection location. The water and the solids are combined in a box opening at the top of a vertical pipe. This avoids the problem of solids passing through a mechanical pump but is only useful when a large supply of water and solids are available at an altitude significantly above the injection altitude. A head box is not useful to supply the usually high pressures of fracture formation as mentioned above for centrifugal pumps

A positive displacement pump enables higher injection depths with higher slurry solids loadings. Piston pumps such as those used to pump concrete and stucco are quite suitable for injection with solids up to perhaps 25 mm in size for very large pumps but more frequently around 15 mm. Progressive cavity pumps are a very good choice if solids are perhaps up to around 10 mm. Progressive cavity pumps can be reversed to pull fluid out of a well while still providing backpressure to the fluid. In this way, they can be used to meter flow out of a pressurized aperture. Still other pumps available to those skilled in the art may prove useful for this purpose.

A pneumatically compressed bladder subsequent to the pump may be a particularly effective check valve variety for trouble-free passage of large solid particles. The pneumatic bladder may be inflated after the pump positive stroke to reseal the subterranean pipe from backflow and the bladder may be deflated to enable the passage of a subsequent charge of slurried solids. This may be carefully and automatically timed for best effect.

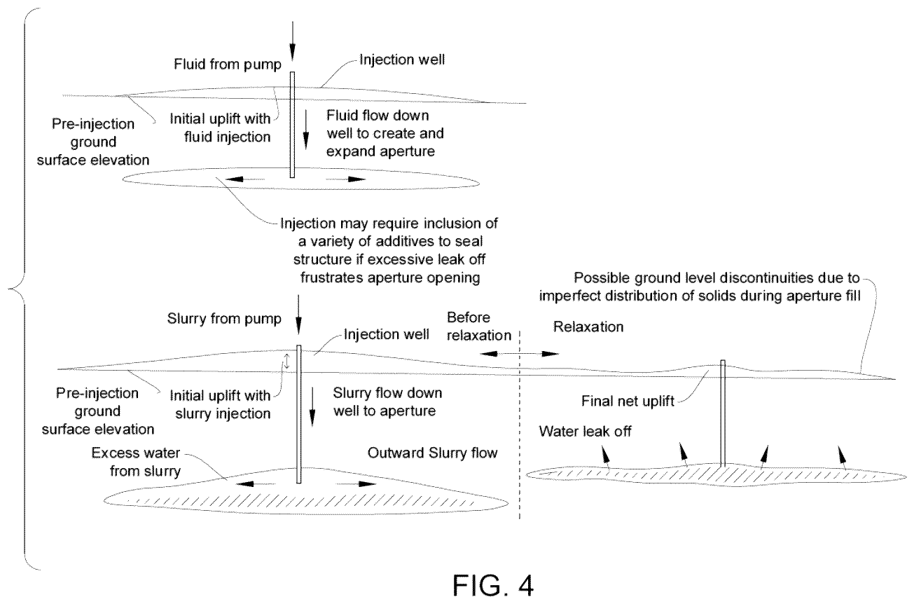

The placement of the slurry may follow a variety of strategies three of which are shown in FIGS. 4, 5 and 6. Option A illustrated in FIG. 4 is a single well, single material placement. FIG. 4 also shows aperture enlargement with fluid pumping to open the aperture size adequately to avoid problems when a primary lignocellulosic solids injection particle size is larger than the initial dimension of the aperture height which may create a solids flow problem. This is the simplest placement strategy and may be used to initiate subterranean lignocellulosic injection operations at a site where plans include multiple well locations and multiple materials for placement. FIG. 4 also illustrates the process of aperture relaxation with leak off of fluid into the subterranean structure. The single well injection strategy typically results in a subterranean orientation of fill materials that is roughly symmetrical about the injection well location as illustrated. Subterranean structures and conditions may substantially alter the material placement away from this generalized symmetrical pattern in any given specific well location.

The first important piece of information that must be understood about any given location is the level of porosity of the geotechnical structure at the injection site. In the extreme case the structure will be so porous that the injection pressure will not rise to indicate that a fracture is forming because the permeability of the injection zone exceeds the capacity of the pumping system at the pressure requirement associated with that depth. Viscosifying agents such as clay are added in Germanovich and Murdoch but an excellent option is fine particle size lignocellulosic materials such as those which may be continuously produced by a dense solids removal hydrocyclone such as that described in slurry formation Option C or D. These fine materials can beneficially reduce water leak-off rates by plugging the pores of the subterranean structure particularly during fluid leak off when the aperture grows quite large.

As an alternative a different variety of lignocellulosic solids may be chosen such as grass and leaves or algae to more efficiently block water escape from very permeable structures. This type of consideration helps inform the material pre-selection step of the slurry placement sequence.

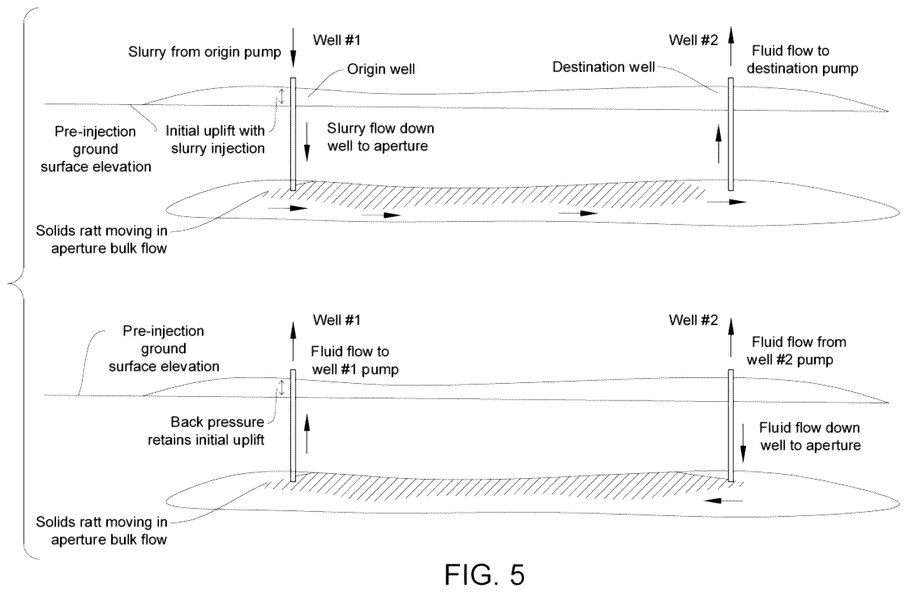

The Option B placement strategy illustrated in FIG. 5 is directional bulk flow with backpressure. As discussed in Germanovich and Murdoch, an array of wells may be used to create a large horizontal aperture under an area where adjacent wells are in hydraulic communication with the same subterranean space. In this way the overburdening earth can rise and fall as a slab in the ideal case. Slurry solids (in Germanovich and Murdoch mineral solids are described) can be sequentially injected into various wells in the array while other adjacent wells are sealed to allow fluid leak off of slurry water through the formation. The substitution of porous lignocellulosic slurry particles enables the ensuing invention of a different way to utilize the array of wells.

As illustrated in FIG. 5 the adjacent well may be used to actively draw the slurry water out of the formation. The lignocellulosic solids of most types will settle or float relatively quickly when flow velocity slows as they move away from the injection well and into the aperture. As they do so a nearly clear liquid space will open toward the center of the flow depth because material will move toward the roof or the floor of the aperture space but away from the middle. A mixed float and sink material will yield an open space in the center between a raft and a pad. A uniformly floating material will result in an open space below the raft. A uniformly sinking injection solid will result in a space above the pad. The floating particles will form more dense entangled rafts and the sinking particles will form more dense entangled pads. In each case the inward most particles will be those that are the most neutrally buoyant and therefore the most easy to dislodge and move horizontally. If the rafts or pads are thickened in areas around the aperture, representing uneven solids fill, the free channel space in these thickened areas will be diminished and the gap will be tighter. Instead of sealing adjacent well-bores as Germanoch and Murdoch propose, with lignocellulosic particles the excess water in the aperture formation can be directly pumped out of the adjacent well by reversing the flow of the progressive cavity screw pumps or switching the check valve directions on piston pumps.

This reversal of the pump direction provides the opportunity to maintain the elevated pressure in the formation using feedback control of the pump flow rates. As the pressure rises at the base of the adjacent well where aperture water is exiting, the exit pump flow rate may be increased to bring the pressure back into the control range. The solids-feed well pressure transducer provides a signal that increases the slurry feed rate as the pressure falls. This enables a bulk flow of slurry to move from the first well to the adjacent well sweeping solids along with it. This increases the control over where the solids move in the formation and how far they may be made to travel. The bulk flow of fluid may be thought of as a fluid rake that both makes more uniform the distribution of solids which have accumulated in thicker rafts or pads and carries solids farther. In the simple central injection radial transport model of aperture fill, the velocity of solid movement in the radial direction falls with the radial distance from the center. In a bulk flow model a stream is created that has near uniform velocity that does not appreciably diminish with distance from the injection well. This uniform and higher velocity sweeps the solids with it.

Instead of relying on liquid leak-off to slowly occur through the formation it is possible to rapidly remove relatively clear water from the formation once the local injection phase is done and the rafts and pads have formed and been leveled by repeated flow and backflow of fluid. Once the introduction of additional solids is stopped, clear water may be injected into each well while sequential adjacent wells rake and spread solids within the space in each of the surrounding directions around the well that was injecting the solids. If for example four wells surround the injection well in a grid. Flow may first be drawn toward well #2 in the figure until excessive solids appear in the #2 well outflow then flow is briefly reversed and well #2 injects clear water to flush the solids back into the formation while well #3 withdraws. Once the well #2 bore is flushed the flow of clear water to well #2 is stopped and well #1 once again pushes water into the formation which is drawn toward well #3 until excessive solids appear. Clear fluid is then pumped down well #3 to clear the bore while fluid is withdrawn from well #4 etc. This sequence of directional sweeping and distribution of solids enables active leveling of the subterranean fill while the aperture is expanded and its pressure is maintained within a defined range which holds the aperture open.

After the sweeping phase is complete the water may be removed from the aperture as the pressure of the formation is rapidly released by slowly drawing water up each well until the pressure falls satisfactorily at each well or excessive solids appear in the fluid at that well. This accelerates the relaxation process for the system of wells.

Directional bulk flow enables wells to place solids toward one side in greater amount. The well can be near the edge of a one-sided filling aperture rather than generally in the middle of the solid fill of an aperture. This improves the ability to demarcate edges of elevated areas more precisely. Solids are swept toward one side of the well by adjacent wells which pull fluid and so direct the flow of placed solids. This is useful for example when a highway is to be elevated but the surrounding terrain is not. It is also helpful to shrink the area of uplift produced on a land parcel and avoid the tilting of adjacent structures which are not to be elevated. Directional bulk flow also enables better economy with injection solids consumption.

Option C, Directional bulk flow with backpressure and material interchange. This option has the same capabilities as Option B with the enhancement that it can inject either a floats-concentrated product or a sinks-concentrated product because a preselection process has created these two available lignocellulosic feeds. As an alternative slurry formation process, Option C produces a concentrated dense (sinks) product and a concentrated buoyant (floats) product, as described earlier. If the initial well, for purposes of illustration, injects floats, the adjacent wells can inject sinks.

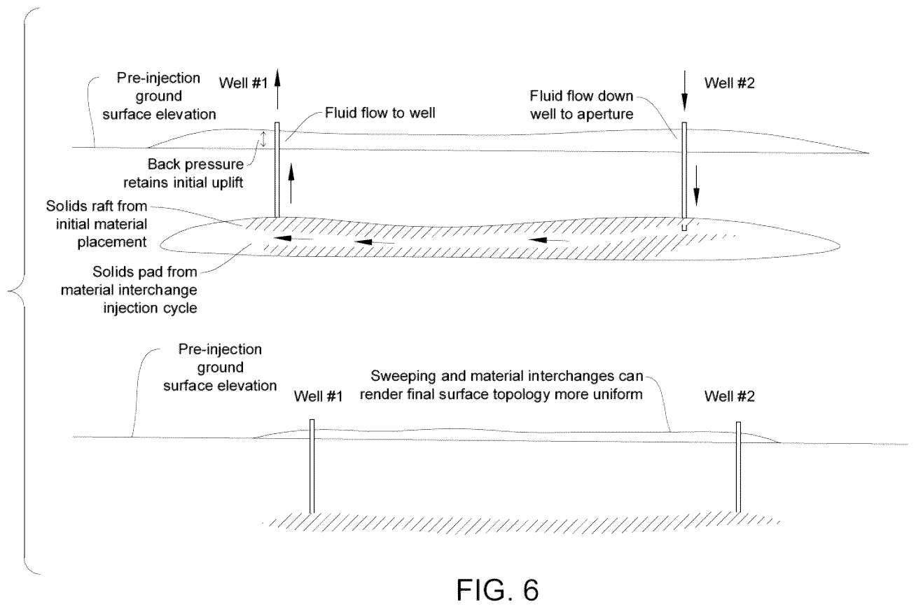

Such a scenario is illustrated in FIG. 6. Floats contain predominantly solids that will form a floating raft in the expanded aperture space and importantly leave a relatively clear path for water to flow toward the bottom of the aperture space. The adjacent wells can inject sinks which have predominantly solids which will form a sinking pad. The alternation of these two varieties of well solids creates the opportunity to more rapidly and efficiently fill the whole height of the aperture with first one material and then finish off the fill process with the alternative missing complimentary material. The fill process can efficiently feather the solids together enabling more rapid fill while still enabling the alternating sweep process to level up the solids placement.

The subterranean injection of lignocellulosic material has substantial novel benefits including: Improvement in the seismic performance of elevated structures, Very long term sequestration of atmospheric carbon which has been incorporated into plant solids, Elimination of fire and pollution risk associated with combustion of plant lignocellulosic material, and Potential Seismic Benefits.

Injection of wood chip materials into the ground at various depths can alter the mechanical performance of the local surface to earthquakes or ground perturbations in a variety of ways that protect structures. Two important mechanisms for structure damage in seismic events are soil liquefaction and transmission of motion to structures. These effects are not significant considerations for lignocellulosic materials placed below 100 m deep in soil because at 100 m or greater depth their significance is outweighed by the influence of the intervening soil but in shallower placements are quite beneficial.

Soil liquefaction in earthquakes results when soils lose strength and stiffness as a result of applied stress. It is mostly observed in water-saturated, loose, sandy soils. The applied stress causes particles of soil to lose contact with one another and the soil water pore pressure to rise. Mechanisms for desaturating soils are described (Cheng Shi et al 2019 Soil Desaturation Methods for the Improvement of Liquefiable Ground IOP Conf Ser.: Mater. Sci. Eng. 562 012015 and Microbe-based Soil Improvement Method JP2012092648A) which discuss methods for introducing gas bubbles in the soil. The gas bubbles can compress during a seismic event as water pore pressure begins to rise and significantly enhance soil resistance to liquefaction. Gas bubbles introduced into the soil structure as described above whether by their presence in the interstices of wood chips or other biomass pores or through the slow decomposition of the wood chips to form CO₂ or methane will also compress in response to rising pore pressure in surrounding saturated soil. This is expected to protect the soil from liquefaction to some extent.

The injection of wood chips into the soil will alter the mechanical characteristics of soil in other ways. Many varieties of biomass are long and fibrous and thus have tensile strength that can be translated to the soil structure. This tensile strength generates confining pressure in the soil to resist loads. Multiple levels of horizontally oriented lignocellulosic layers would be expected to reduce the soil movement in a horizontal dimension such as might be caused by the placement of a high vertical load on the column of soil. Wood chips are compressible and can rebound if stress is reduced. If a time variable and high level of stress is applied normal to planar mass of wood chips the compression of the chips would be expected to alter the maximum stress level transmitted to the soil or rock on the opposite side. If the stress is applied at a frequency, the presence of the springy wood chip plane might be expected to alter the frequency of the stress transmitted across the plane under many circumstances.

A saturated porous body of wood chips enables movement of water in response to variations in soil stress. The presence of vapor space within the wood chips can enable small local movement of water to compress the trapped vapor instead of moving the stress freely through the soil or rock structure. Also, the wood chip body can allow small movement of water toward lower resistance regions for example upward movement of water in a vertically oriented plane of wood chips. This enablement of movement introduces a level of viscous dissipation to the soil or rock.

The fundamental mechanical character of the ground structure beneath a construction can be altered with these characteristics in mind. The strategic application of wood chip layers in different orientations such as vertical, horizontal, inclined, cupped or bent can represent one variable to be engineered. The stacking of these planes or shapes in any given dimension can create intricate distributed reinforcement. The porous layer of wood chips may be used to provide a protective channel through which water is directed around, underneath or away from an area. If initial injections form vertical apertures, these may be filled with solids and thereby increase the mechanical character of the ground by increasing the level of horizontal stress in the region. Subsequent injections may thus more preferentially form in a horizontal dimension. The thickness of the layer or in various parts of a given layer and the variety of biomass within regions will materially alter the stress and strain behavior and porosity of a body of wood chips. This may be thought of as adjusting the spring constant of the ground for various applications of stress. The center of a layer may be of one character while the periphery is of a different character. The viscous dissipation character and the dimension within which the dissipation is most pronounced may also be thoughtfully adjusted. The quantity of sequestered vapor which plays an important role in enabling dissipative movement of water and of the ground may be adjusted by selecting different types of wood chips (biomass) whether by selecting those which possess more isolated vapor or those types which decompose to a saturation level of vapor and thus renew any vapor that may be lost with time. A very small addition with time of additional nutrients, oxygen or microbes (as partially described by Cheng above) may also be used to tune vapor inclusion or regeneration.

The ground structure can thus be tuned in a variety of ways to protect structures from frequencies of ground movement to which those structures are most vulnerable. The frequency, direction and intensity of stresses applied by seismic events to structures may in these ways be engineered. The ground may be designed to be most protective of planned or existing structures. A building or structure may thus be tuned in conjunction with its ground structure to provide the most cost-effective protection from seismic ground movement or liquefaction or from damage caused by the movement of water within the ground such as that which can cause sinkholes. This may all be done at the same time that other aspects of the area such as its surface elevation are changed.

Controlling the extraction of reaction products as mentioned above can be used as a mechanism to regulate decomposition rate and the formation of new gas bubbles. In addition to adding oxygen, or required nutrients such as fixed nitrogen or phosphorus may be expected to maintain desirable gas bubbles in a subterranean wood chip area to enable continued protection from ground movement or rapid increase in pore water pressure.

Using this combination of benefits, areas may be simultaneously protected from rising sea level or subsidence of land below sea, lake or river levels as well as from ground movement events and liquefaction of soil. Elevation of areas protects from rising relative water levels while altering soil mechanical nature gives additional protection from ground movement such as earthquakes.

Hypothetical situations will be described that show how the disclosed techniques may be preferentially used to best effect under imagined conditions. The information provided is supported by experimentation with the various materials, literature values for subterranean structure information and equipment related knowledge and experience. The preferred embodiment depends on a wide-variety of site-specific conditions and goals and so the detailed decision making methods described in the specification yield different preferred choices for different sites.

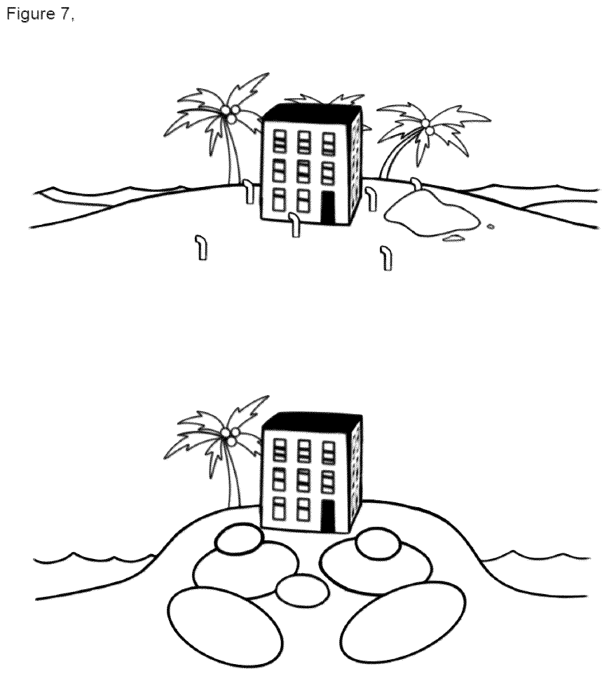

An island resort, illustrated in FIG. 7 is facing inundation due to sea level rise and there is no available mineral fill material locally. A large nearby lagoon hosts extensive algal growth due to local use of fertilizers to grow food and coconuts. The lagoon also serves as a catchment for some floating plastic debris which is unsightly. The decaying algae washes up daily contaminated with fine floating plastic in large moist piles and creates noxious odors as it dries and decays in the sun.

It was decided to elevate the resort by 1 meter over a period of 10 years with a combination of the two most abundant lignocellulosic biomass materials that are available: algae and coco. The sequence of steps provided in FIG. 1 was followed to guide the process. First, the approximately three hectare space around the resort was selected for elevation. Then, the starting elevation of the resort was 0.3 m with a uniform level grade. Next, the geotechnical profile of the area includes a relatively uniform partially cemented coral rubble (conglomerate) with cemented beach sand (beachrock) and sand. There is an underlying lithified reef at a depth of 10 meters. Based on this information, elevation apertures at a depth of 20 meters were used to elevate the three hectare yard of the resort. Ten (10) meter deep apertures were used at the interface between the lithified reef and the cemented coral conglomerate sand profile to ensure precise leveling of the resort building which occupies 1,000 m².

Next, four injection locations were selected for the 20 meter deep wells at a radius of 49 meters from the center of the resort along the center of the resort's North, East, South, and West profiles. Four additional well locations were selected for the 10 meter deep wells at a radius of 18 meters from the center of the resort in the center of the Northeast, Southeast, Southwest, and Northwest profiles.

The eight wells were drilled and a 100 mm well pipe was cemented and sealed in place with a capillary pressure transducer placed at the base of each pipe to allow accurate measurement of aperture pressure. A level controlled tank on one edge of the yard supplied seawater for well slurry preparation and injection. A batch slurry preparation area for the partially dehydrated algae which was collected from a beach on the lagoon at a distance of 150 meters from the resort was used for the 20 meter injections. A common piping system for the four algae wells was buried in a shallow trench running to each well. A second coconut and cocopalm grinding area 150 meters from the resort was utilized to supply the four shallow wells close to the resort building. These wells were also joined with a piping system run in a trench. A manual well selection system for each well type could supply pump pressure to any given well while sealing the three others.

A 20 MPa high pressure jet pump was used with a rotatable pressure pipe to score the lithified reef stone at the base of each 20 m well in a 360° arc to a radius of 50 cm to initiate the aperture. The jet pipe with the nozzle removed was then temporarily placed with a removable pressure packer at the base of the hole to protect the well piping from high pressure. The base of the well was pressurized with the jet pump using a pressure relief at a maximum pressure of 3 MPa. The lithified reef stone began to crack as the pressure was slowly elevated and as the crack opened the aperture formed at the base of each well. The jet pump hardware and packer were removed.

No jet pump was needed with the 10 m wells because the apertures were to be located at the interface of the lithified reef stone and the sand and partially cemented conglomerate layers. The progressive cavity pump was used to pressurize the structure but it was found that a high level of leak off occurred on the structure initially. The algal material which forms a thick paste when the water content was reduced was pumped into each well and quickly sealed the leaky subterranean structures enabling sufficient pressure to begin opening the apertures at the base of each 10 m well.

Progressive cavity positive displacement pumps were used to pressurize each well system and expand the apertures. Surface altimeters and tiltmeters were used from this point forward to monitor the topology of the surface in preparation for placement of lignocellulosic materials in the apertures.

Slurries were prepared for the 20 m wells with a batch slurry preparation in agitated tanks because the partially dried algae with some fine plastic debris and sand readily formed a thick slurry suitable for placement according to FIG. 3 Option A, to feed the progressive cavity pumps. No grinding or sand and plastic debris removal was done to preselect the slurry material. The algae material was used in a sufficiently thick state to eliminate floating and separation within the piping system which reduced the need to clean and flush piping. The thick algae slurry was quite stable at a 20% by volume solids content. An elevation of 100 mm per year was targeted for the surface above the 20 m wells and a schedule of injection was chosen that allowed each of the four wells to be injected every 2 weeks with a net elevation of 8.3 mm each month for each well.

Slurries were prepared for the 10 m coconut wells with a centrifugal concentrator with the hydrocyclone as detailed in FIG. 3 Option C. The floats/coarse product returned to the grinder and the hydrocyclone fine/dense product which was well saturated with water delivered to the 10 m wells. It was also important to ensure that the product sent to the wells was well saturated with water to ensure that materials would not saturate with water within the piping system and swell in place or bind up as free water was drawn into the slurry particles. The system operated using seawater and a mixture of 50% by volume milled coco palm frond and wood with 50% milled coconut shells and husks. The particle size was 3 mm or less. The dilute slurry pump drove the centrifugal floats concentrator and the hydrocyclone.

A progressive cavity pump delivered the prepared slurry to the wells at about a 12% solids content by volume. Monthly injection was done because the particle size of the material required larger spaces for penetration so lifts of less than 14 mm did not yield good solids flow. Elevation was done as required to maintain the building level with the elevating yard and avoid unacceptable tilting or differential elevation that might damage the building.

The slurry piping was rinsed after each injection cycle for both wells.

The release of excess water from the algae wells required more than a week and a settling of about 75% occurred which required that the initial lift of ground surface every two weeks was about 15 mm. Release of water from the coco wells required only several hours and a settling of about 50% occurred which required about 16 mm of elevation to achieve a net 8.3 mm of lift monthly.

An assessment was done of level changes due to elevation and settling and planning of future injections was done accordingly. The yard area was covered with a 40 mm layer of coco mulch to control weeds and provide ample habitat for methanotrophic bacteria which would oxidize methane release primarily from decomposition of a portion of the algae. There was less methane emitted from the coco fill around the structure as planned since coconut has a very high lignin content and degrades much less anaerobically than does algae.

A San Francisco Bay area highway was built on ground constructed after the 1906 earthquake by filling in a portion of the bay. It crosses a portion of a meandering old stream bed that ran through a salt march into the bay. The highway is particularly subject to damage from seismic soil liquefaction and lateral spreading. This area has undergone extensive subsidence and with rising sea level faces inundation routinely several times a year during king tide or storm events. It was decided to elevate the highway.

It was decided to elevate a 300 m long section of the two lane highway 30 m wide by 1 meter over a period of one year. Using 6 mm and under fir bark fines available from the California forestry industry. The sequence of steps provided in FIG. 1 was followed to guide the process.

First, the approximately one hectare, 30×300 m rectangular space beneath the roadway was selected for elevation.

Second, the starting elevation of the highway was 0.3 m with a uniform level grade.

Third, the geotechnical profile of the area includes a relatively uniform dredged fill to a depth of 5 meters over a sandy consolidated bay mud profile that extended to 30 meters, followed by a cemented mudstone layer to a depth of 50 meters Based on this information, elevation apertures at a depth of 30 meters were selected to intersect with the mudstone interface.

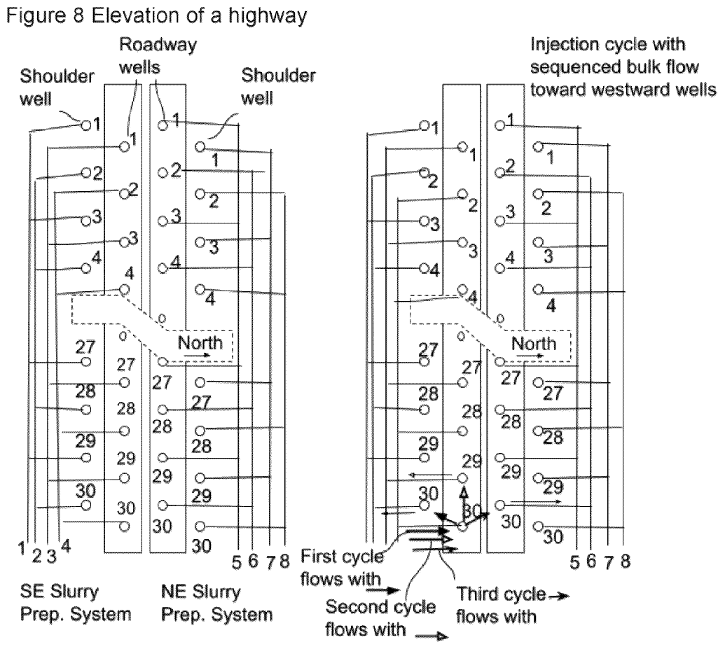

Fourth, the fill strategy would require relatively frequent injections of high uplift thickness. A grid of 30 wells on four lines spaced 10 meters apart on a 5 meter staggered grid was selected. A total of 120 wells would be required. The close spacing of the wells was necessary to ensure that by pressurizing the wells the roadway could be lifted as a slab to avoid local bending that might fail the paving surface. The well layout along the roadway and example slurry injection bulk flow sequence are shown in FIG. 8.

Next, the 30 wells on each of the two outside lines were drilled vertically while the 30 wells along each of the two central lines were drilled at an angle from the roadway shoulder in order to avoid shutdown of freeway operation during the project.

Eight pipes were run along the roadway shoulder to manifold the wells. The first pipe manifolded the odd numbered wells on the southern shoulder vertical wells. The second manifolded the even numbered wells on the southern shoulder vertical wells. The third pipe manifolded the odd numbered inclined wells under the southern lane. The fourth manifolded the even numbered inclined wells under the southern lane. The fifth manifolded the odd numbered inclined wells under the northern lane. The sixth pipe manifolded the even numbered inclined wells under the northern lane. The seventh pipe manifolded the odd vertical wells on the northern shoulder. The eighth pipe manifolded the even vertical wells on the northern shoulder.

A progressive cavity pump was installed on each of the eight lines that could run in either forward direction or reverse direction. Each well had a separately actuated pneumatic valve. These wells were also joined with a piping system run in a trench. A sophisticated automatic well selection system could supply pump pressure to any given well while sealing all the others wells along that particular manifold line. All solids were supplied from either a Northeast or Southeast slurry forming station.

Next, the apertures at the base of each well were initiated with only pressure from the progressive cavity pumps because the subsoil interface above the mudstone profile facilitated the crack. The sandy consolidated bay mud profile provided excellent formation sealing so excessive leak off was not encountered.

Next, progressive cavity positive displacement pumps were used to pressurize each well sequentially from East to West and expand the apertures. Surface altimeters and tiltmeters were used in grooves cut in the pavement from this point forward to monitor the topology of the surface in preparation for placement of lignocellulosic materials in the apertures.

Next, slurries were prepared for the wells using a centrifugal concentrator with the hydrocyclone as detailed in FIG. 3 Option C. Slurry placement technique Option C similar to that shown in FIG. 6 is used. The floats/coarse product was fed to the inclined wells under the roadway with the initial placements for the roadway wells moved by bulk flow toward first one westward well, then a second westward well and finally a third westward well. Subsequently the hydrocyclone fine/dense product which was well saturated with water was delivered to the vertical shoulder wells and swept sequentially first to one westward well along the shoulder, and then a second westward well under the roadway. There were two slurry preparation systems on the Northeast shoulder and one on the Southest shoulder.

The fir bark had few binding problems in the piping and so the less saturated float product worked quite well despite incomplete saturation with water. The system operated using baywater. The dilute slurry pump drove the centrifugal floats concentrator and the hydrocyclone.

A progressive cavity pump delivered the prepared slurry to the wells at about a 20% solids content by volume. Injections were done every week. During each injection cycle the floats material was first injected under the lanes while the 3 adjacent wells westward of that well were used to sequentially withdraw water to sweep the fill material first toward the Southwest, then toward the West, then toward the Northwest. After this the sinks material was injected in the shoulder well and the two adjacent westward wells were sequentially used to withdraw fluid. In this way the heavy material was allowed to flow in the gaps left after the floats product was injected under the lanes. This accelerated the process of filling the aperture and increased the penetration of the material by virtue of the bulk flow. The time required to relax the wells was also reduced.

Next, the slurry piping was rinsed after each injection cycle for both wells.

Next, the release of excess water from the fir fines wells required several days and a settling of about 50% occurred which required that the initial lift of ground surface every week was about 38 mm. Because the elevation occurred in a linear stretch along the road no excessive cracking occurred in the pavement surface.

Then, an assessment was done of level changes due to elevation and settling and planning of future injections was done accordingly. The road shoulder and slope were covered with a 40 mm layer of fir bark mulch to control weeds and provide ample habitat for methanotrophic bacteria which would oxidize methane release primarily from decomposition of a portion of the fir. There was little methane emitted from the fir bark fill around the roadway because fir bark has a very high lignin content and degrades very little anaerobically.

The roadway elevation project provided a degree of base isolation to the highway which reduced the transmission of seismic energy to the highway. The increased vapor bubbles created by the slow decomposition of the fir bark migrated upward through the shallow dredge fill profile which was most vulnerable to liquefaction as well as upward through the sandy bay mud. The presence of these bubbles reduced the tendency for soil pore pressure to rise with seismic activity and so reduced the likelihood of liquefaction of the soil.

The description of the different advantageous embodiments has been presented for purposes of illustration and description, and is not intended to be exhaustive or limited to the embodiments in the form disclosed. Modifications and variations will be apparent to those of ordinary skill in the art. Further, different advantageous embodiments may provide different advantages as compared to other advantageous embodiments. The embodiment or embodiments selected are chosen and described in order to best explain the principles of the embodiments, the practical application, and to enable others of ordinary skill in the art to understand the disclosure for various embodiments with various modifications as are suited to the particular use contemplated.

In one arrangement, a mechanism for providing variable back-pressure to the subterranean aperture is provided. For example, a hydrocyclone style separator or other similar centrifugal solids separation device on the flow of liquid exiting the underground space may be utilized. As just one example, a hydrocyclone style separator will provide a variable back-pressure that increases with liquid flow rate. The diameter of the hydrocyclonic separator and its inlet and outlet sizes may be altered to adjust the amount of pressure required to drive a given flow through the unit. In this way, a level of back-pressure may be used that is adequate to maintain the size of the subterranean aperture.

In one preferred arrangement, the formula for the requisite volume of flow that is delivered to the subterranean aperture includes make-up water. This make-up water may be utilized to account for a volume of fluid that may be lost from the subterranean aperture to the surrounding soil structure. In order to maintain a constant aperture volume the following formula may be satisfied.

$$Constant {V}_{aperture} , {V}_{liquid in} = {V}_{formation loss} + {V}_{liquid out} - ( {V}_{solid in} - {V}_{solid out} ) \qquad Equation 1$$

At the start of a project or elevation, the volume of the aperture must increase and at the end of an injection process, the aperture's volume will decline as the additional liquid added escapes. In addition, the deposited solids are compressed by the mass of the overburden of soil above. While solids are being added to the aperture, the added solids will displace liquid that was added to increase the size of the aperture.

Directional bulk flow of material in one well location while removing liquid from another area of an aperture will result in some solids leaving the aperture with the exiting fluid. Thickeners and viscosifying agents will be present in this flow if these agents were used in the injection fluid. These agents are valuable to recover and fluid reuse enables this recovery while at the same time avoiding the creation of a surface waste fluid. The exiting fluid will contain fine material such as sand and clay removed from the subterranean structure by action of the fluid. This solid material derived from the soil structure abutting the aperture may be returned to the aperture space by recycling.

The continuing addition of fluid and withdrawal of fluid in the directional bulk flow solids placement strategy will cause the level of fine particle size solids which have a long settling time to accumulate in the transportation fluid until their concentration reaches an equilibrium value. This accumulation of fine solids forms an autogenous thickening agent which can complement the thickening agents applied at the surface. This enables reduction of the total requirement for surface addition of thickeners.

Sodium bentonite, calcium bentonite and polymeric thickeners have been observed by others to reduce the tendency of introduced water to destabilize fine grain soil in vertical or horizontal tubular wells. No information was available to characterize their performance in stabilizing horizontal inverted planar surfaces. This would be an important benefit to their use when a horizontal soil aperture is opened because the inverted horizontal planar roof or upper surface of the subterranean space has a tendency to absorb the aperture water and swell. This swelling and water uptake causes destabilization of the aperture roof structure. This can cause a continuing collapse as the soil particles on the roof gain water, loose cohesion and detach to sink to the floor of the open aperture structure.

Techniques of improving aperture roof stability were investigated. A roughly 12% by weight 1.065 g/cc density sodium bentonite slurry was placed in contact with a piece of inverted planar dried modeling clay. A 1000 ppm solution of anionic polyacrylate was similarly contacted with a piece of this clay. These were compared to the performance of a similarly configured piece of dried modeling clay in contact with tap water. The tap water very rapidly caused the clay to form fine particles, which exited like snow falling from the inverted particle and collapsed its structure.

The anionic polyacrylate solution reduced the rate of the clay particle collapse, requiring more than twice the time of the tap water.

The sodium bentonite slurry did not collapse the clay but instead partially hydrated and softened it over a much longer period of time. After agitating the anionic polyacrylate solution with the clay particle the clay particle broke apart completely and was more strongly suspended in the liquid than were clay and water alone.

The combination of clay and anionic sodium polyacrylate resulted in a more stable and pumpable clay suspension than water alone yielded. It was concluded that either bentonite or anionic polyacrylate would provide some protection to collapse for the roof of the aperture by slowing water infiltration.

It is also likely that the related polymer anionic polyacrylamide which is used as a commercial sealant for ponds would also perform well in this regard and by a similar mechanism to anionic polyacrylate. Aperture opening with a bentonite slurry or more concentrated anionic polyacrylamide may suffice to enable longer term sealing whereupon a larger volume of liquid with much less sealant and carrying lignocellulosic solids may be introduced.

Injection of a gas into the subterranean aperture is an excellent way to protect the roof from direct exposure to water that can cause clay to lose cohesion and fall from the roof. The injection of a gas stream may be done by blending with the entering liquid stream or as a separate stream into the aperture space. This gas may then collect at the upper surface and partially shield the roof from exposure to penetrating water. It is desirable to limit oxygen exposure over the long term to the chips which can accelerate degradation. If the gas stream is enriched in nitrogen and thus depleted of oxygen this is advantageous. Air itself is a nitrogen-enriched gas and the roughly 21% oxygen will rapidly be depleted if no new oxygen is supplied once the aperture is sealed. The active compression of the aperture by anchor devices may serve to eliminate much of this gas before aperture sealing.

Tests were performed to verify that the presence of thickeners makes it easier to transport solids to the aperture and stabilize the bounds of the aperture but the tests showed these thickeners also increased the settling time required to deposit lignocellulosic solids. The background for the testing, the rationale for selection of thickeners to be tested and then the tests themselves will be described.

A first requirement to be tested is whether a given thickening system can be pumped. The novel active deposition process here described as directional bulk flow introduces a second requirement that the lignocellulosic solids will accumulate in the aperture. The settling time for the solids must therefore be higher than the time required to transport the fluid with the solids from the aperture entry to the aperture fluid removal location.

Pulp fiber that has been subjected to a lignin removal operation often for incorporation into paper is an attractive thickening agent. It is widely available at low cost and may be sourced in the form of recycled paper or cardboard and repulped in wet-blending devices which are known to those skilled in the art. Our laboratory testing of pulp fibers suspended by blending mixed recycled office paper in a Vitamix 5000 blender. at various concentration in 400 ml beakers with a Brookfield viscometer using a #2 RVT spindle at 10 and 100 rpm are given in Table 1 below where 2% pulp concentrations begin to exceed acceptable viscosity of around 3000 cp at low shear rates. At concentrations under 0.1% even microcrystalline cellulose pulp particle sizes do not sufficiently thicken slurries to 10 cp. 0.1% SigmaCell type 38 from Sigma Chemical was tested on a Brookfield viscometer with RVT #2 spindle at 100 rpm to give 12 cp. and at 10 rpm to give 16 cp.

Microcrystalline cellulose is quite expensive to purchase and so coarser pulp fibers such as are used in conventional paper may be more cost-effective. Pulp fiber that has been reduced in lignin content has the advantageous property of shear thinning for easier pumping but also gives a minimum yield stress to enable solids settling. A wood chip of insufficient size or buoyancy differential from the fluid will thus not settle if the fluid has a high yield stress to enable particle movement. This creates the possibility of a non-settling slurry which greatly facilitates free slurry flow avoiding clogs and screen-outs which block flow. This is particularly advantageous when injection without fluid removal from an aperture is desired because bulk slurry flow to an aperture discharge location places a limit on the minimum settling rate to avoid lignocellulosic material exiting the aperture.

TABLE 1
Paper pulp viscosity at low and high shear rates

| Mixed ofc. Pulp % | 10 rpm | 100 rpm |
|---|---|---|
| 2 | 3800 | 200 |
| 1 | 1560 | 94 |
| 0.5 | 140 | 48 |

Table 1 illustrates that pulp fiber slurries have different viscosities at different shear rates. The shear rate in a centrifugal separation device is high and so the lower viscosity of pulp at 100 rpm shear rates is more relevant. When a fluid is pumped into an aperture the higher viscosity at 10 rpm is more comparable. This creates a surprising advantage that can be exploited because pulp fibers when used as a thickener can be subsequently removed by a centrifugal separator from the fluid for potential reuse. The pulp solids settle more quickly with the lower apparent viscosity in the higher shear environment. A pulp-containing viscous slurry can be injected at one location of the aperture to expand and shape the aperture and stabilize the aperture roof with clays, polyacrylate or polyacrylamide sealants. This same slurry can be removed at a different or multiple different locations once the shape of the aperture is perfected. The pulp thickeners can be passed through a hydrocyclone or other centrifugal separator while back-pressure is maintained to ensure the aperture stays open.

The fluid can be returned to the aperture with a different viscosity that is lower once some of the pulp has been removed by the separator. This fluid then has a lower viscosity that is designed to allow deposition and settling of lignocellulosic materials that are now introduced and suspended in the slurry. Polysaccharide thickeners can't be easily removed and must be either diluted, chemically broken apart or discarded. A more viscous fluid is desirable during the aperture formation and expansion process but a less viscous fluid is needed later to enable lignocellulosic solids deposition in the aperture so that settling rates are not too high.

Cellulose pulp concentrations of up to 18% by weight form pumpable slurries with levels of 2-15% by weight pulp forming an excellent slurry for transporting solids. The cellulose pulp itself can form the bulk of the carbon sequestered in the subterranean space or can be used to transport a second perhaps lower cost or more recalcitrant solid such as wood chips. Combinations of quantities of guar gum comprising 1% or less of the slurry augmented by wood pulp or other lower cost thickeners is a particularly advantageous approach. A target range of 0.2-1% guar gum, xanthan gum or equivalent carbohydrate thickener with 1-5% cellulose pulp is an attractive and effective option for transporting solids in slurry.

The use of cellulose pulp thickener with fluid extraction after aperture shaping while aperture shape is maintained by back-pressure enables both solids and liquids to be separated and recovered independently for reuse. The aperture forming fluid viscosity may be high while the same fluid may be used with a lower viscosity later for deposition of solids. Polysaccharide gums such as guar or xanthan gums are known to increase the viscosity of fluids but they are costly. It was discovered that by using a combination of 1% pulp fibers and 1% guar polysaccharide gum, a synergistic benefit appeared enabling stable suspension of a lignocellulosic material more than 50% of which did not pass a 6 mm screen. 1% pulp fiber from recovered cardboard was used in these tests but mixed office paper appeared to perform similarly once pulped. A 1% pulp fiber with only 0.5% guar gum adequately suspended a lignocellulosic material 50% of which did not pass a 5 mm screen.

Visual observation of laboratory slurries is believed to be among the more reliable ways of showing adequate slurry performance. A Brookfield viscometer could not reliably gather data on large particle slurries of this type. Similarly a Marsh cone does not reliably pass solids of this size.

Addition of more than 10% by mass bentonite clay solids content to a 10% lignocellulosic slurry yielded a slurry that was judged thicker than would be required in combination with a lignocellulosic placement but useful before placement to assist in opening an aperture.

A centrifugal separation device such as a hydrocyclone accelerates deposition of coarse solids including sand and coarse lignocellulosic materials. Bentonite clay thickeners are shear-thinning and thus their apparent viscosity declines when they pass through a high shear environment inside a hydrocyclone. As fluid is recirculated from the surface to the aperture and back to the surface in the solids placement operation a hydrocyclone may be used to alert operators that one of several conditions will require adjustment: the deposition fluid viscosity may require reduction to expedite lignocellulosic settling rates, the size or degree of water saturation of the incoming prepared lignocellulosic materials may require increase, or alternatively additional exit wells locations might be simultaneously used so as to increase the areal fraction of the aperture through which liquid passes. Increasing the number of exit wells used increases the apparent area of the aperture and thus increases residence time for settling of particles.

An additional alternative to reduce the population of coarse lignocellulosic particles in the exiting flow is to reduce the total fluid entry and exit flow rates.

The location of the active aperture determines the area where solids deposition underground will occur. The balance of forces in the subsoil space determines the location where the aperture will form or persist. These forces are partially determined by the initial stress state of the soil. In one arrangement, these forces may be adjusted by altering the stress state of the soil.

A crack can form when the forces that are normal to the dimension of the crack holding the soil together reach zero at the edge as the crack propagates. Aperture geometry may be actively altered by a system that changes the force magnitude or direction with time in different locations.

As an example, if a fluid-filled aperture existed beneath a roadway and a heavy vehicle moved over that fluid-filled aperture from one edge to another of the aperture, the shape of that aperture would be expected to change in response to the changing load that the vehicle represented.

Many apertures do not open in the desired direction or expand so rapidly outward that adding volume does not increase the aperture height adequately to enable large chips to be injected without fear of plugging.

One method that might be utilized for resolving this potential rapid expansion problem is to increase the injection fluid viscosity. Fluid mechanics study tells us that viscous fluids experience a higher pressure drop when flowing through a distance than do less viscous fluids. Applying this understanding to the problem of expanding the height of an aperture to accept larger particles such as a lignocellulosic material with greater than 50% by mass not passing a 5 mm square screen, it is clear that a by increasing this fluid viscosity, a high pressure drop will be maintained as fluid moves away from the injection port and towards the periphery of the aperture. This creates a high pressure near the injection opening while a much lower pressure is experienced at the leading edge of the expanding fracture. Such a scenario may be created since the high viscosity results in a rapid reduction of fluid pressure as fluid moves outward and away from the injection site.

The viscous fluid can optionally contain some combination of nearly 50% by mass fine mineral material whether from recycle of fluid exiting the aperture or from addition of up to 12% bentonite clay. The fluid may also contain from 40-1000 ppm anionic polyacrylate, 0.1-2% cellulose pulp subjected to lignin removal operation, or polysaccharide gums at a concentration of 0-1% for example guar or xanthan gum. The use of any of these viscosifying materials solely or in combination can achieve the goal of enabling the injected material to act as a fluid jack to increase the local height of the aperture to accommodate lignocellulosic solid particles wherein more than 50% by mass of the material does not pass a 5 mm square opening screen.

There are a number of advantages to be gained by employing a diversity of viscosifying agents. Tests were run on two different varieties of natural viscosifying agents including grape pomace and citrus pomace. The term “pomace” is used to describe a residual material usually created by squeezing a desirable juice or oil from a fruit or seed raw material. Pomace materials often have natural thickeners, for example fruit pectin. Damp grape pomace was placed in a high intensity hydropulper and agitated with an equal volume of water for 20 minutes. The resulting blended product contains solid seeds, grape husks and water and is thick and directly pumpable as a solid containing slurry. It can be mixed in at a range of concentrations from 30% solids content down to around 1% solids content or lower. The grape pomace may be directly blended with wood chips to transport wood solids in a thick slurry. A favorable ratio of 5-20% solids grape pomace with 0-5% pulp fiber and 0-40% wood chip or sawdust material on a solids mass basis in water is quite acceptable. Orange pomace created by squeezing juice from orange peels was blended to form a 9% solids paste in a water slurry using a high intensity mixer. This orange or other citrus material would be a very effective slurry thickener alone or in combination with other thickeners at a solids fraction of 10% down to around 1%. Natural and synthetic film type materials also could be used as a slurry thickener to transport solids. Examples of natural films would be onion skins, chopped grasses or equivalent and synthetic plastic bag film would serve the same purpose. Waste plastic film may thus be chopped to 30 mm or less in size and become a valuable thickening agent to transport woody or other biomass materials into underground areas.

An additional thickener which would introduce no contaminants would be frozen water (ice) slurries. Ice slurries can be made stable using freezing point depression additives such as soluble salts or sugars. The presence of a freezing point depressant in the liquid lowers the tendency for the residual fluid to solidify and creates a stable fluid with ice particulate as is known to those skilled in the art. This type of thickening system may be very advantageously used when there is saline or brackish groundwater in the injection zone underground. Alternatively soluble sugars can be used and extracted for reuse in more fluid slurries by the techniques described in other parts of this discussion. A water and ice slurry was blended in the laboratory and an ice fraction of between 10 and 60% forms a slush capable of suspending biomass solids alone or in combination with other types of thickening agents such as guar gum, cellulose pulp or pomace materials.

In certain arrangements, it may be possible to affect the shape of the underground aperture without the use of expensive viscosifiers by applying forces on the surface layer of ground or by applying these forces subterraneously. This can be done in a number of ways including by the use of large weights, anchored plates, and cables.

Large weights placed in specific locations on the top layer of soil affect aperture shape. For example, weights can be placed around an injection in a ring pattern. The force exerted on top of the soil from these weights increases the pressure necessary to inject material through the volume of soil lying under the ring pattern at the injection depth. This can create an aperture with a higher vertical to horizontal displacement ratio due to the injected material aligning in a vertical column due to the resistance generated by the external pressure created by the weights.

Application of a load at the surface such as from a heavy truck or by filling large water tanks or piling soil above a space are ways to adjust the subterranean forces affecting the aperture. However, there are a number of other ways to adjust the forces affecting aperture location and shape.

For example, a vertically oriented anchoring device such as a shaft, tube, cable or other similar structure may pass through the upper portion of the soil profile and be anchored in the ground beneath the plane of an existing, proposed or possible aperture zone. Applying a tensile force to the vertical oriented anchoring device creates a compressive load downward on the space where the aperture zone might be and provides a closing force on this space. The vertical distance between the points of application of the lower and upper reaction forces determines the areal extent in the horizontal plane of the soil zone influenced by the applied load as is understood by the science of Soil Mechanics. Anchoring devices of various types both above and below the zone to be under compression may be used including soil nails, augers and anchoring plates that pivot to anchor into soil when tension is applied. An additional variety of soil anchoring device is a cemented anchor where the cement is applied to the zone of the soil to which the load may be beneficially applied in either tension or compression. The cemented anchor may include a ring or hook embedded in the cementitious layer which is accessible after the cement cures and to which a connection mechanism to transfer force may later be attached and detached at will. Using an anchor below and above the targeted depth for injection where the anchor below that zone is pulled upward and the anchor above the zone is pulled downward. The closer these two anchors are in a vertical dimension, the more concentrated the force is to seal off a smaller area. If the injection level is at 10 meters deep, it may be advantageous to locate one ground closing anchor 2 meters below that depth at 12 meters deep and the upper anchor point which provides downward force 2 meters above the injection zone at 8 meters deep. The next ground sealing anchor assembly might be around 4 meters away horizontally along the desired line of force being created with the intention of fencing in the ground elevating injection. Spacing anchor assemblies along a horizontal line at approximately the distance apart to match the distance that the center of the upper and lower anchors are spaced apart vertically is advantageous.

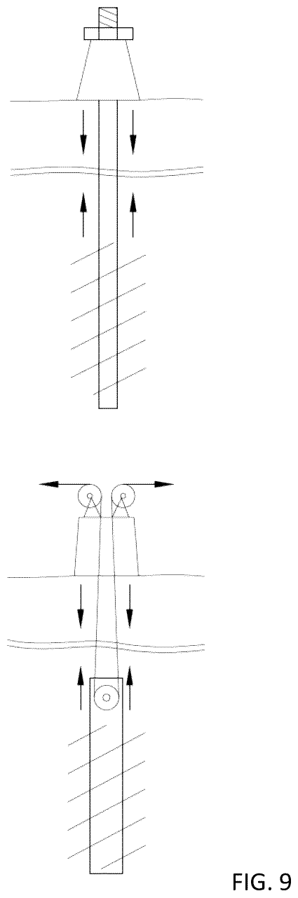

One anchoring method that may be used is to drive a single large auger into the ground with a threaded section on the superterranean portion of the auger central pipe or cylinder. A platform or plate can then be made to apply pressure to the soil as a nut is tightened on the threaded section as depicted in FIG. 9A.

FIG. 9B depicts an alternative technique employing a cable as the tensile member creating the compressive force. In one arrangement, the cable may be inside tubing to avoid contamination of the cable and pulleys with soil. A single cable may pass down to pulleys underground or a stub cable or chain connected to the subterranean anchor may rise to the surface where a surface mounted tensioning arrangement may attach to it. The surface mounted device for applying tension to the vertically rising stub cable or chain is thus protected from contamination by soil. A cable has the capacity to apply tension to a number of such soil anchors at once using a single cable winch as an actuator. They can also be placed with precision similar to the weight method described earlier.

Additionally, a sequence of anchoring systems like cables or screws for applying force have the advantage of “sewing” the ground together as there is an upward force applied from the anchoring auger and a downward force from the superterranean tightening mechanism.

For example, a single 3,000 kg cable winch can create 6,000 kg compressive force on each anchored cable because there would be 3,000 kg on each of two cables passing to the subterranean anchor. If the same cable passed through 20 cable anchors in a line stretching over a 100 m, it would create 1,176 kN force or the equivalent of parking 5 fully loaded 24 metric ton trucks in a line at the push of a button. Other varieties of techniques could be used to compress screw type or hydraulic soil force application systems automatically.

A 1,1176 kN force applied along a 100 m line, arc, or circle will likely stop an aperture from opening and also close one along the line that is already open. This enables careful shaping of elevated spaces and the dynamic flow of solids within a filled or filling aperture.

These methods for applying force enable compressive forces to be applied in a line to “fence” in an area. In this way, the periphery of a subterranean aperture may be defined so as to effectuate control of the aperture's growth, shape and extent in a horizontal plane. Horizontal control of the aperture area enables an increase in aperture height when additional liquid is pumped into the aperture. The hydraulic pressure of the pumped fluid opens the aperture but within the constraints imposed by the anchors. Without limiting the horizontal growth of the aperture, additional liquid pumped into the aperture may increase areal extent without meaningfully increasing aperture height.

In one arrangement, two small augers can be driven in the ground on either side in a horizontal plane of an anchoring plate with a threaded rod above it. Connecting these and then tightening a bolt downward against the anchoring plate causes a downward force on the plate. This increases the pressure on the volume of soil underneath the plate which force is translated to an upward tension on the two horizontally adjacent augers in the soil. This particular method does not involve perforating the soil directly in the area where the downward force is applied but instead perforating the soil some distance to either side horizontally of the compressive force application.

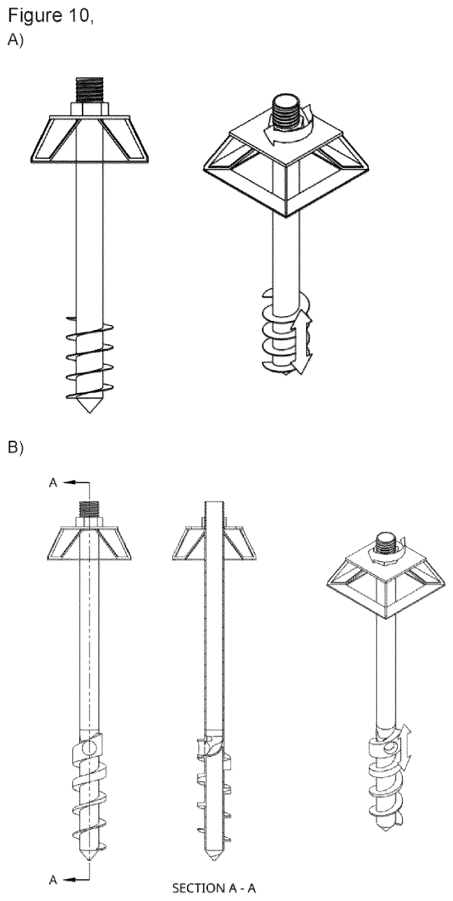

FIGS. 10 (A) illustrates in more detail a compressive soil anchor and 10(B) shows a soil anchor with a slurry injection discharge at the upper end of the auger anchor that enables rotation to clear plugs which may form when injecting biomass materials. The placement of the subterranean discharge port normal to the axis of the anchoring device assists in reduction of plugging and enables higher fluid velocity in a selected direction. The addition of a liquid pressure jet operating at 7-38 MPa assists in pushing the injected slurry forward if applied such that the jet impinges on the fluid after the solids have begun moving outward from the axis of the well and into the aperture. The jet may be used to clear plugged material and assist cleaning along with the rotating normally oriented discharge port mentioned earlier.

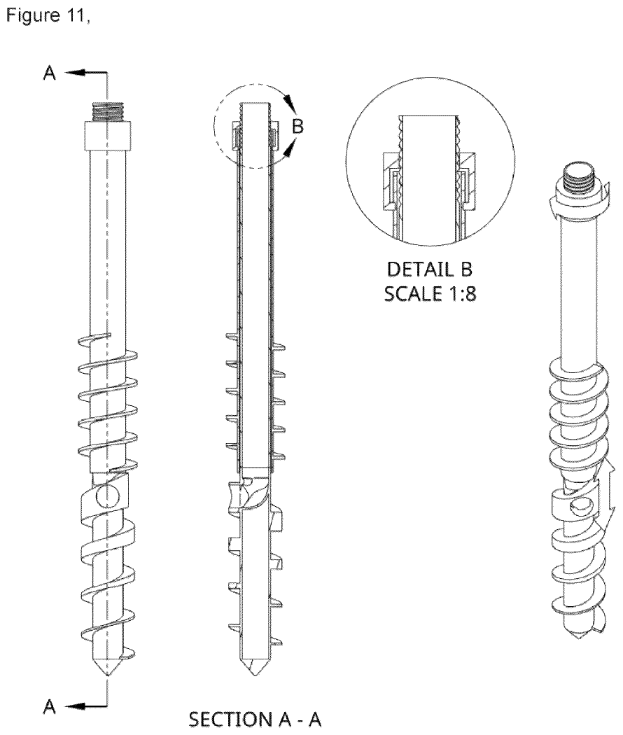

FIG. 11 illustrates a soil anchor that does not employ a surface mounted footing to locate the upper reaction force but instead a second concentric auger soil anchor which may be located at any distance between the soil surface and the lower soil anchor by a separate rotary placement operation or a cementitious placement. In this way, the location of application of the reaction force may be altered in the vertical dimension. Both a compressive force and an opening or tensile force may be created between the upper and lower soil anchors by employing any of a variety of methods to create differential movement of the lower and upper soil anchors.

A second soil anchor such as an auger which can be placed a precise distance above a lower anchor enables application of compressive and tensile forces to alter the tendency of apertures to either open or close. Tensile forces encourage opening and compressive forces encourage closing or reduce the tendency to open. At one moment an area of subsoil can be in compression and moments later after adjustments are made to the mechanism, it may be in tension or the level of compression may be dramatically reduced relative to surrounding soil.

Dynamic aperture shaping is thus enabled as well as direct movement of aperture fluids from one area to another area without pumping fluid in or out of the aperture at the surface.

The dynamic opening and closing of one or many locations using an array of soil anchor devices such as those illustrated in FIG. 10 or 11 create opportunities to both control where and to what height apertures open and when and where fluid will be flowing within an aperture. Flow may be driven by pumping fluid in from the surface or pumping fluid in at one surface location and allowing or pumping it out a second or at multiple surface locations.

Removal of fluid is accomplished while maintaining back-pressure to support the aperture height. This can be done using a reversible pump such as a progressive cavity or a peristaltic pump whose rate is adjusted to maintain adequate back pressure or by a hydrocyclone device which increases back-pressure intrinsically as flow rate increases. An additional way for this to occur is to discharge fluid from within the aperture out of exits whose altitude above the ground may periodically be adjusted but which will intrinsically provide back pressure to the exiting fluid flow based on that discharge altitude. Each of the methods just described for metering exit flow enables passage of solids including those of up to 10 mm or more in a dimension.

Without surface fluid movement to or from the aperture, fluid may be transferred around the aperture by sequential compression of areas of the aperture. This is conceptually similar to squeezing a tube of toothpaste to move the toothpaste around inside the tube.

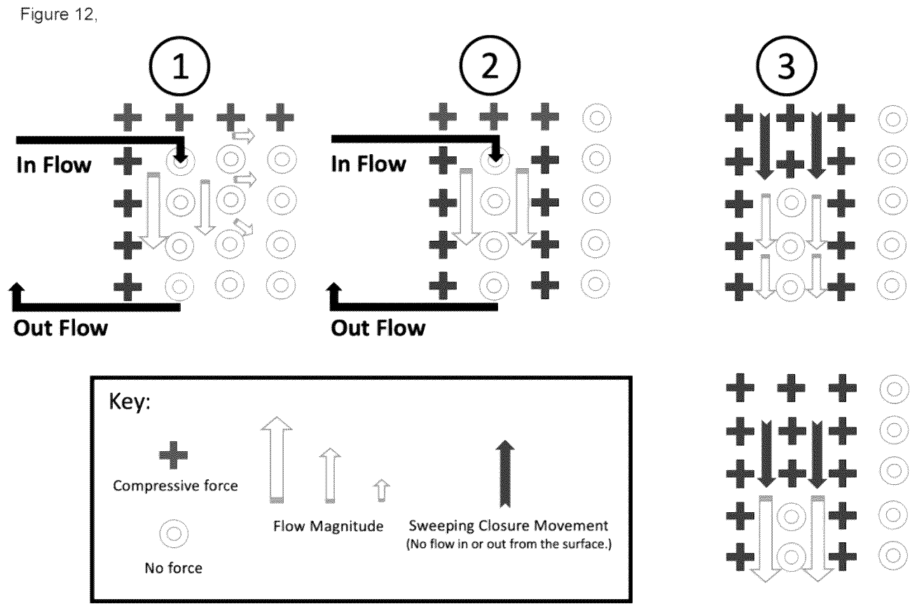

The use of anchoring mechanisms to induce fluid flow is illustrated in FIG. 12. In column 1 of FIG. 12 an aperture is bounded on two sides by compressive anchors that restrict flow but still allow some light flow to the right. Column 2 illustrates the impact of a third bounding side which narrows and strengthens flow downward. The anchors on the three bounding sides preclude the fluid flow in these three directions and constrain the aperture as depicted in column 2. In column 3 the external flow to the aperture is stopped and a sweeping closure movement is induced by applying compression at first one and then sequentially a second location moving downward. This drives liquid flow from within the aperture space and may help to both drain and level the intra-aperture solids toward the unbounded lower edge. Column 1 would have a longer solid settling time than column 2 because a larger area of the aperture is experiencing flow. The higher rate in column 2 might, for example, lead to an unacceptably high proportion of the dense solids exiting the aperture without deposition.

If the fluid inside the aperture is a slurry, the solids may move with the induced flow. If the solids have settled, the supernatant fluid may move in this way so as to ease its removal or recycling to transport more solids to the aperture.

Removal of supernatant fluid from the aperture enables its recovery and reuse. This recovery is accomplished by direct addition to the injection pump sump. The recovery of valuable viscosifying agents such as guar gum, sodium bentonite and cellulose pulp is also accomplished in this way.

The complete leak-off of extra fluid from the subterranean aperture will require about 1 week. Thus it is best to first measure the elevation achieved by injection at least one week after injection is ceased. The minimum elevation achieved with solids 50% of which do not pass a 5 mm square screen opening will be about twice the median 5 mm size or about 10 mm.

Packers are designed to facilitate the injection of materials (usually grout, epoxy, or cement) into subterranean formations or structures through controlled, pressurized means. This saves time and money by allowing for injection pipes to be reused instead of being cemented or glued in place before injections. Packers are composed of an inner pipe usually made of steel or stainless steel and an expandable sealing element, which with hydraulic actuation, securely isolates the target zone, ensuring precise grout placement and also optimal dispersion. The hydraulic actuation causes a bladder to swell. This bladder is either so elastic that it can swell enough to seal against the borehole, or the actuation causes a slip to move up the packer which allows it to swell enough. The packer's mechanism allows for repeatable, high-pressure deployments.

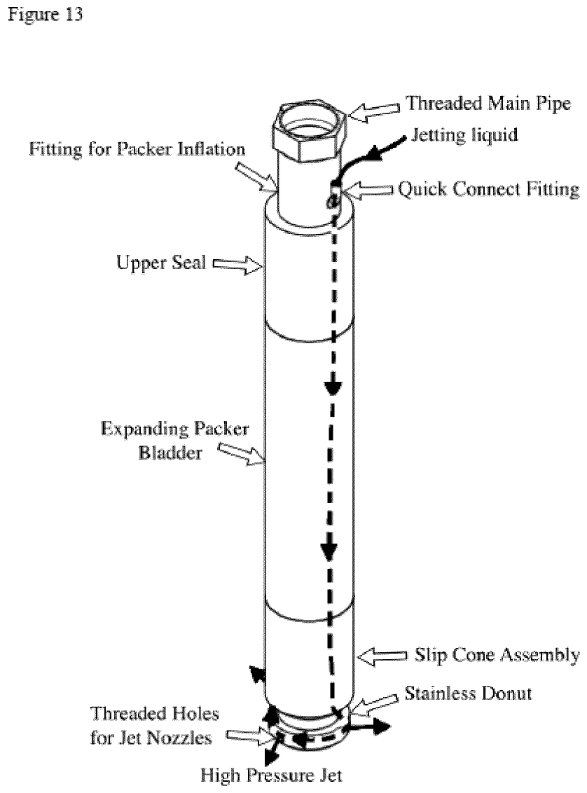

The fracker packer is a variation of a hydraulic packer that is optimized for wood chip and wood pulp slurry injection. FIG. 13 depicts a fracker packer. Fracker packers are generally larger than those used in grouting. They start with a passthrough diameter of 1″ and can be as large as 12″ in diameter. Typically, the outer diameter is between 150% and 200% of the pass through diameter. Fracker packers main difference from traditional packers is the internal tube that connects to one or multiple jets. The internal diameter of this tube is sized off of the required flow rate for the jets and the thickness is sized off of the pressure required for the jets. Usually, the path for the jet starts with a quick Connect female fitting that is parallel to the main pipe and far enough under the threaded section of the main pipe that its male counterpart can be coupled. Quick Connect fittings are preferred as this allows pressure washers or triplex pumps to be connected with off-the-shelf hoses. This fitting is welded to the inner tube which goes through the wall of the main pipe at a 45 degree angle and is welded to seal it. This tube then goes along the inner wall of the main pipe and exits the main pipe after the packer's slip or sealing section. It is welded flush to the main pipe and covered by a hollow donut that is either welded or contains internal O-rings. The internal O-ring structure allows the donut to be spun by the jets of liquid or to rotate the jets by hand or using a device at the surface. This hollow donut either has one or many orifices drilled directly in it for the jet of liquid, or it has one or many threaded holes for typical pressure washer nozzle tips to be threaded into. Threads are preferable as jet spray angles can be varied.

This fracker packer technology allows for the delivery of high pressure liquid below the hydraulically sealed bladder. This jet of liquid is primarily used to cut an initial aperture in the soil under the packer. The resulting cut is shaped like a bicone without tips and is centered on the plane at the vertical location of the jets or angled downward at around 0-30 degrees. This is an ideal shape to begin fracturing soil horizontally with the hydraulic pressure of subsequent injections. The longer the high pressure liquid is jetted, the deeper the cut and the larger the volume of the aperture. The larger the volume of the aperture, the lower the maximum injection pressure for subsequent injections. When the liquid coming up the fracker packer and the pipe or hose connected to it has no more visible particulate, further jetting will have reduced efficacy. It is important to space the fracker packer up above the bottom of the borehole as some material that is jetted out is too dense to be pumped up without specialized drilling fluid and will settle at the bottom of the borehole.

The fracker packer also allows for upstream clogs and slurry screen-outs to be reversed back up the main pipe. Water liquid delivery to the bottom of the packer prevents a vacuum and allows material to be removed. This is especially important when pumping biomass slurries as biomass is frequently not homogenous and contains oversized chips and other particulate contaminants. Pressure sensors are attached in line with the packer inflation hose, high pressure jet hose, and the pipes or tubes going to the main pipe to monitor this process and the slurry injection process. Additional sensors and instruments such as strain gauges can be secured to the fracker packer for additional data collection and real-time control.

It is appreciated that the optimum dimensional relationships for the parts of the invention, to include variation in size, materials, shape, form, function, and manner of operation, assembly and use, are deemed readily apparent and obvious to one of ordinary skill in the art, and all equivalent relationships to those illustrated in the drawings and described in the above description are intended to be encompassed by the present invention.

Furthermore, other areas of art may benefit from this method, and adjustments to the design are anticipated. Thus, the scope of the invention should be determined by the appended claims and their legal equivalents, rather than by the examples given.

### Section II: Modeling and Control

The present system design is divided into two facets: automation of the planning of sites and the automation of our injection and associated procedures. These systems demonstrate interconnectivity as models contained within one are affected by the performance of the other. Additionally, the site planning subsystem directly interacts with the general behavior of the control system. Here, we describe the design of both subsystems and subsequently, their cross interaction.

### 1. Site Planning System

The site planning subsystem is designed to generate automatic comprehensive guidelines of the optimal methodology for subterranean slurry injection for elevation, sequestration, or other focuses.

### 1.1 Input Data

In order to gain a comprehensive computational model of the geospatial composition of a given site, we utilize data gathered from numerous sensor modules. These include but are not limited to:

Land Survey: This input involves the collection of precise geographical data to map the surface features of the site. It provides foundational information for understanding the layout and physical characteristics of the land, crucial for initial planning and alignment with other subsurface data.

Electrical Resistivity Tomography (ERT): ERT data is used in identifying subsurface material composition by measuring electrical resistivity. This non-invasive method helps in detecting variations in moisture content, porosity, and material type, offering valuable insights into the subsurface structure.

Ground Penetrating Radar (GPR): GPR is utilized for its ability to detect and map subsurface features using electromagnetic waves. It is particularly effective in identifying geological layers, voids, and other anomalies, thereby assisting in the assessment of suitable drilling locations.

Seismic Refraction: This technique involves measuring the travel time of seismic waves to interpret subsurface geological structures. It aids in determining the depth and characteristics of various layers, which is crucial for understanding the geomechanical properties of the site.

Reflection Surveys: Similar to seismic refraction, reflection surveys use seismic waves but focus on the waves reflected from subsurface structures. This method provides detailed imagery of subsurface layers, enhancing our understanding of the geological context.

Electromagnetic Surveys: These surveys measure the earth's electromagnetic fields to infer the subsurface conductivity and magnetic susceptibility. This data is vital in identifying different types of rocks and minerals and in mapping the subsurface distribution of these materials.

Gravimetric Surveys: By measuring variations in the Earth's gravitational field, gravimetric surveys help detect density changes in the subsurface materials. This input is significant for identifying voids and cavities or denser materials like ores underground.

Geotechnical Drilling: This method involves drilling into the subsurface to collect samples for laboratory analysis. It provides direct information about the soil and rock properties, which is essential for assessing the feasibility and safety of drilling and injection processes.

Soil Sampling: Soil sampling and testing are essential for gaining comprehensive insights into soil composition, texture, moisture content, and crucially, its physical properties. This includes specific tests to determine mechanical properties such as tensile and shear strength, along with the state of consolidation. These assessments not only aid in understanding how soil interacts with injected slurry but also predict the behavior of the subsurface during and after injection. Additionally, these tests serve a dual purpose: they assess the potential for preservation of lignocellulosic materials and evaluate factors contributing to groundwater protection. This information is vital for determining site suitability for various surface uplift strategies and understanding the overall environmental impact.

Geocore Sampling: The practice of geocore sampling, where cylindrical sections of soil or rock are extracted, is crucial for delving into the subsurface's stratigraphy and mechanical properties. This method is fundamental in revealing the underlying structure and composition of the soil, providing essential data that informs our understanding of the subsurface environment.

Topsoil Testing: Testing is conducted to specifically examine surface soil characteristics. This aspect of soil analysis is vital for identifying surface tensile properties, which are key to understanding and predicting soil cracking events. Furthermore, assessing the methane oxidation potential of the soil through topsoil testing is critical for environmental impact studies and carbon management strategies.

Vegetation Analysis: Incorporating data about trees and surface plants into our soil analysis model significantly enhances our predictions regarding soil behavior. Understanding the impact of vegetation, particularly in areas devoid of the root-based tensile reinforcement, is essential for accurately forecasting soil cracking. Moreover, the influence of vegetation on soil properties like methane oxidation, water absorption, and density is invaluable for guiding ecological decisions. This might include recommendations for biochar application or strategic planting of specific tree species, like oaks, to optimize environmental benefits and soil management.

Property Valuations: An estimation of property valuations of those located atop the site and the adjacent plots is useful in computing potential damages and thus the incentive/disincentive for models to select specific land for different purposes.

### 1.2 Topographical Modeling

Utilizing the comprehensive data inputs outlined, our system generates two primary models: geospatial topology and surface topology.

For the geospatial topology system, our objective is to generate a mesh grid, which serves as the foundational structure for mapping and analyzing geospatial and hydrological data. This mesh grid is articulated through two primary embodiments, each offering a unique approach to integrating and understanding subsurface data.

The first embodiment, which involves a mesh grid with layers of subterranean material and attributes, is constructed through either manual input or advanced computational methods. In the manual process, users systematically input collected geospatial data into a predefined three dimensional mesh grid structure. This mesh collection may be constructed in multiple ways including but not limited to Regular Grid Representation, Irregular Grid, Triangle Mesh, Voxel Representation, Point Clouds, Octree, Bounding Volume Hierarchies, and Sparse Voxel Octrees. For purposes of illustration, here, the mesh points can be mathematically represented in a regular grid representation as:

$$( {x}_{i} , {y}_{j} , {z}_{k} ) for i = 1 , 2 , … , n ; j = 1 , 2 , … , m ; and k = 1 , 2 , … , p$$

where xᵢ, yᵢ, and zₖ represent the coordinates in the horizontal, vertical, and depth dimensions, respectively.

Alternatively, machine learning algorithms streamline the integration and analysis of various subsurface datasets. Supervised learning models, once trained on historical geospatial data, develop the capability to discern intricate patterns and correlations among different types of subsurface data. By leveraging these patterns, the models can effectively synthesize diverse datasets into a comprehensive mesh grid. This not only extends our understanding of the subsurface layers beyond direct measurements but also enhances the accuracy of the geological and hydrological interpretations.

In contrast, unsupervised learning algorithms approach the data analysis from an independent standpoint. These algorithms employ advanced techniques like kriging or spline interpolation to construct the mesh grid. Kriging, a geostatistical method, provides a way of interpolating values at unmeasured locations, using a weighted average of known values:

$${Z}^{*} ( {x}_{0} ) = {\sum }_{i = 1}^{n} {λ}_{i} Z ( {x}_{i} )$$

where Z*(x₀) is the estimated value at the location x₀, Z(xᵢ) are the known values at locations xᵢ, and λᵢ are the weights calculated based on the spatial correlation between known points and the estimation point.

Spline interpolation, on the other hand, fits a smooth curve through the known data points. This curve minimizes the overall curvature, providing a smooth transition between points. It can be represented in a simplified form as:

$$S ( x ) = {\sum }_{i = 1}^{n} {a}_{i} {B}_{i} ( x )$$

where S(x) is the spline function, aᵢ are the coefficients, and Bᵢ(x) are the basis functions.

These interpolation methods are instrumental in training the models to identify attributes at varying depths and coordinates, culminating in a coherent mesh grid that accurately represents the subsurface structure. To ensure the consistency and accuracy of this layered structure, a similarity score matching algorithm may be employed. This algorithm functions by comparing the attributes of each layer, computing a similarity score based on predefined metrics, and then matching layers that exhibit high degrees of similarity. The computation of this similarity score can vary based on the attributes and the specific requirements of the analysis, but it generally involves assessing the Euclidean distance or a cosine similarity measure between attribute vectors.

In an extended embodiment of our models, we introduce the concept of a mutability coefficient, a factor used to assess and quantify various geological and environmental attributes of a region. The mutability coefficient is a multifaceted measure, capturing the capacity of a region to undergo specific types of changes or interventions, such as uplift, permeability, cracking tendency, and/or ease of drilling, as well as others.

In this context, mutability may refer to the ability of a region to experience uplift or deformation, often due to subterranean injections. This aspect of mutability, akin to plasticity in materials science, quantifies the extent to which the subsurface layers can be molded or altered without fracturing. Mutability may also encompass the region's permeability, specifically regarding water. This aspect measures how easily water can pass through the subsurface layers, a vital factor in assessing flood risks, irrigation potential, and groundwater movement. Another important facet of mutability may be the tendency of the terrain to crack or fracture, typically in response to external forces or environmental changes such as changes in surface tensions. Mutability can also refer to the ease with which drilling operations can be conducted in a region, indicating the resistance offered by the terrain to such interventions.

To quantify mutability, statistical methods and ML models are employed. The mutability coefficient is determined as a weighted sum of various attributes that signify different aspects of mutability. This calculation is achieved using multivariate regression, a statistical technique that models the relationship between multiple independent variables and a dependent variable. In our case, the independent variables are the attributes (like plasticity, permeability, etc.), and the dependent variable is the mutability coefficient. The mathematical representation of this relationship can be expressed as:

$$M = {β}_{0} + {β}_{1} {X}_{1} + {β}_{2} {X}_{2} + … + {β}_{n} {X}_{n}$$

where M is the mutability coefficient, β₀ is the intercept, β₁, β₂, . . . , βₙ are the coefficients for each attribute X₁, X₂, . . . , Xₙ.

ML models, particularly those using reinforcement learning, may also be applied to iteratively adjust these coefficients. These models simulate the outcomes of slurry injection and drilling, learning from each iteration to refine the coefficients for better accuracy. Alongside these computational methods, manual tuning is an integral part of determining the mutability coefficient. This involves adjusting the coefficients based on empirical data and observed responses from actual sites. The process of manual tuning allows for the incorporation of real-world complexities and nuances that may not be fully captured by statistical models alone.

The first embodiment of the surface topology involves similar methods to that of the geospatial topology system. In this embodiment, manual input might involve using topographical maps and survey data to create a basic grid layout, while ML-based methods utilize algorithms to integrate surface data, gathered through mentioned sensor readings, to construct the grid.

An additional embodiment may involve using algorithms to determine the influence of subsurface conditions on the surface topology. Statistical correlation techniques or ML algorithms trained to predict surface topology changes based on subsurface data variations are employed. Furthermore, Computational Fluid Dynamics (CFD) and Finite Element Analysis (FEA) simulations are used to simulate physical responses of the surface to subsurface changes. ML algorithms specialized in pattern recognition and prediction analyze the data generated by these simulations to refine the mesh grid.

In a particular embodiment, such as those used for sequestration-focused sites, the congregation of the information outputted by the geospatial and surface topology processes is considered a comprehensive geospatial analysis and output by the site planning system. In sequestration-focused sites particular emphasis is also placed on methane transport and oxidation prediction, as detected and computed through aforementioned sensor inputs. In other embodiments, namely for elevation purposes, this information is automatically digested into another pipeline which we describe below.

### 1.3 Injection Purpose Embodiments

For the purposes of elevation, the system first determines the ideal end-state topography that the process of injection should mutate the current terrain into. In some cases, the system does this through interpreting the purpose of the injection. Here, for instance, we consider flooding, flattening, and terraforming system usages as different driving purposes.

Flooding: The first embodiment involves querying a specialized application programming interface, (API) designed to provide real-time or historical data on flood ratings for different topographies. This data is typically derived from a range of sources, including satellite imagery, elevation data, weather forecasts, and hydrological models. The flood rating provided by the API can be a numerical value or a categorical rating, indicating the likelihood or severity of flooding in a particular area.

### 1.3.1 Flood Risk Embodiment 1

Building upon this, we may train a learning system to refine the flood risk assessment based on the API readings. This system may employ supervised learning algorithms, which, after being trained on a dataset comprising various topographical features and corresponding flood histories, is adept at recognizing complex patterns and correlations. The ML system analyzes features such as historical rainfall expectation, soil composition, land cover, and historical flood data to make accurate inferences about flood risks on different types of terrain. The training process involves optimizing a loss function that measures the difference between the predicted flood risk and the actual observed outcomes. This could be represented mathematically as:

$$L ( θ ) = \frac{1}{N} {\sum }_{i = 1}^{N} {( {y}_{i} - f ( {x}_{i} ; θ ) )}^{2}$$

where L(θ) is the loss function, N is the number of data points, yᵢ is the actual flood risk, xᵢ is the input feature vector, ƒ is the model, and θ represents the model parameters.

### 1.3.2 Flood Risk Embodiment 2

Another approach within our process utilizes numerical methods, such as calculating the elevation difference between the ground and nearby water bodies. This method is based on the principle that areas with lower elevation relative to nearby water sources are generally at higher risk of flooding. The elevation difference can be calculated using digital elevation models (DEMs) and can be mathematically represented as:

$$Δ h = {h}_{ground} - {h}_{water}$$

where Δh is the elevation difference, h_{ground} is the elevation of the ground, and h_{water} is the elevation of the nearest water body.

To enhance our flood risk assessment model, we consider the dynamic nature of water elevation (h_{water}(t)) influenced by a combination of environmental factors. The comprehensive model for water elevation is formulated as follows:

Atmospheric Pressure Influence (Δhₐₜₘ(t)): The change in water elevation due to atmospheric pressure is represented by

$$Δ {h}_{atm} ( t ) = - \frac{{P}_{atm} ( t ) - {P}_{0}}{ρ g}$$

where Pₐₗₘ(t) denotes the atmospheric pressure at time t, P₀ is the reference atmospheric pressure, ρ the density of water, and g the acceleration due to gravity.

Wind Stress Influence (Δh_{wind}(t)): The impact of wind on water elevation is captured by

$$Δ {h}_{wind} ( t ) = {C}_{wind} \cdot {V}_{wind}^{2} ( t ) \cdot cos ( θ - {θ}_{wind} ( t ) )$$

with C_{wind} being a coefficient, V_{wind}(t) the wind speed, θ the coastline orientation, and θ_{wind}(t) the wind direction at time t.

Precipitation Influence (Δh_{precip}(t)): Water elevation changes due to precipitation are given by

$$Δ {h}_{precip} ( t ) = {C}_{precip} \cdot {P}_{rate} ( t )$$

where C_{precip} is a coefficient and rate (t) the precipitation rate.

River Discharge Influence (Δh_{discharge}(t)): The contribution of river discharge to water elevation is

$$Δ {h}_{discharge} ( t ) = {C}_{discharge} \cdot {Q}_{river} ( t )$$

with C_{discharge} as a coefficient and Qᵣᵢᵥₑᵣ(t) the discharge rate.

Sea Level Rise (Δh_{sea_level}(t)): The long-term sea level rise effect on water elevation is modeled as Δh_{sea_level}(t)=β·t, where β indicates the rate of increase over time.

Tidal Influence (Δh_{tidal}(t)): This factor includes both the primary and additional tidal components, represented by

$$A sin ( ω t + ϕ ) + {\sum }_{i = 1}^{n} {A}_{i} sin ( {ω}_{i} t + {ϕ}_{i} )$$

where A and Aᵢ are the amplitudes, ω and ωᵢ the angular frequencies, and ϕ and ϕᵢ the phase shifts.

Integrating these factors, the extended model for calculating water elevation is expressed as:

$${h}_{water} ( t ) = {h}_{mean} + {\sum }_{factor} Δ {h}_{factor} ( t )$$

In this model, Δh_{factor}(t) denotes the contribution from each factor atmospheric pressure, wind stress, precipitation, river discharge, sea level rise, tidal influence, and ocean currents—to the overall water elevation at time t.

### 1.3.3 Flood Risk Embodiment 3

An alternative embodiment uses a combination of the aforementioned approaches. The system may extract learned features relevant to flood risk assessment from historical data, such as soil composition, land cover, and previous flood events. These features are among those that capture the complex interplay of factors that contribute to flood risk. Alternatively, the system can calculate geographical and physical features using numerical methods. For instance, utilize DEMs to compute elevation differences between the ground and nearby water bodies, highlighting areas potentially at risk due to their lower elevation.

In this embodiment, we may train a supervised ML model (e.g., Gradient Boosting Machines, Neural Networks) on a dataset that includes both the features derived from historical patterns and the features calculated using numerical methods. This combined feature set enriches the model's input, enabling it to learn both from the patterns in historical data and the immediate geographical realities represented by numerical calculations. Furthermore, we may modify the loss function of the ML model to incorporate penalties or rewards based on the numerical method calculations. For example, predictions that disregard high-risk indicators from the numerical features (such as significantly lower elevations near water bodies) could incur a higher loss. This encourages the model to pay attention to critical geographical factors alongside historical patterns.

Let's denote the ML features as x_{ML} and the numerical method features as XNA, with the combined feature vector represented as c_{combined}=[x_{ML}; x_{NM}]. The model's prediction function can then be expressed as ŷ=ƒ(x_{combined}; θ), where θ represents the model parameters.

The loss function, incorporating both ML and numerical method insights, can be represented as:

$$L = \frac{1}{N} {\sum }_{i = 1}^{N} ℓ ( {y}_{i} , f ( [ {x}_{MLi} ; {x}_{NMi} ] ; θ ) ) + λ \cdot R ( {x}_{NMi} , θ )$$

Here, N represents the number of data points, l is the primary loss function measuring the discrepancy between actual flood risks yᵢ and predictions ŷᵢ, and R is a regularization term or an additional component of the loss function that integrates insights from numerical methods, with λ controlling its influence. This term can penalize predictions that ignore critical geographical risk factors identified by the numerical methods. This approach offers a more flexible and holistic model of flood risk.

Once the flood risk is determined using these methods, the next step is to identify the topography of the terrain that optimizes certain parameters—minimizing flood risk while also considering other critical factors like required vertical displacement, cost, injection time, disruption to the terrain, and interference with adjacent properties.

For example, to quantify the interference with adjacent properties, we introduce a sophisticated interference function, I(x, y, z), which is incorporated into the overall optimization framework. This function is expressed as a sum of various factors, each with its specific weight and exponent, allowing for a representation of different types of interference:

$$I ( x , y , 𝓏 ) = {\sum }_{i = 1}^{n} {w}_{i} \cdot {F}_{i} {( x , y , 𝓏 )}^{{p}_{i}}$$

where wᵢ are the weights, Fᵢ(x, y, z) are the individual interference factors (such as distance to property boundaries, elevation impact, hydrological impact, etc.), pᵢ are the exponents providing non-linear scaling, and n is the number of factors considered.

To account for the hydrological dynamics of water distribution within the specified area, we may include a new term within our sophisticated interference function, I(x, y, z), to specifically address the distribution of a fixed quantity of water—resulting from rainfall and runoff from adjacent areas—across the terrain. The modification involves considering how the volume of water distributes over a sub-region, factoring in the area's elevation difference relative to a defined plane of highest water level (HWL). This concept is critical for understanding the impact of terrain modifications on local hydrology and adjacent properties.

Accordingly, the interference function is augmented to include a hydrological interference factor, F_{hydro}(x, y, z), that calculates the impact of elevation changes on water distribution. This factor is defined as follows:

$${F}_{hydro} ( x , y , 𝓏 ) = \frac{{V}_{water}}{{A}_{sub - region} \cdot Δ {𝓏}_{HWL}}$$

where V_{water} is the fixed quantity of water delivered to the area, A_{sub-region} is the area of the sub-region under consideration, Δz_{HWL} is the elevation difference of the sub-region below the plane of the highest water level. Incorporating this into the overall interference function, we have:

$$I ( x , y , 𝓏 ) = {\sum }_{i = 1}^{n} {w}_{i} \cdot {F}_{i} {( x , y , 𝓏 )}^{{p}_{i}} + {w}_{hydro} \cdot {F}_{hydro} {( x , y , 𝓏 )}^{{p}_{hydro}}$$

where w_{hydro} and p_{hydro} are the weight and exponent assigned to the hydrological interference factor, respectively. This addition allows the interference function to dynamically reflect changes in water distribution due to terrain elevation adjustments, ensuring that the optimization process accounts for the resultant hydrological impacts.

Flood risk-based approaches may, therefore, involve a multi-criteria optimization problem, where we aim to find a solution that balances the various mentioned factors, including the newly formulated interference function. The optimization can be formulated as:

$${min}_{x} \{ F ( x , I ( x , y , 𝓏 ) ) \vert x ∈ X \}$$

where F(x, I(x, y, z)) is a multi-dimensional function representing the different factors (flood risk, cost, time, disruption, interference, etc.), and X is the set of all possible terrain configurations. The optimization process might employ techniques like gradient descent, genetic algorithms, or linear programming, depending on the complexity and nature of the problem.

Flattening: The system focuses on computing an approximately flat terrain based on a strategically chosen point of highest importance. This process is pivotal in various applications, including construction, landscaping, and urban planning, where terrain flatness is a critical factor.

The selection of the point of highest importance is a crucial initial step in this process. Ideally, this point is identified as the location with the highest elevation (z-value) within the given site area. Mathematically, if we consider a set of points P={(xᵢ, yᵢ, zᵢ)|=1, 2, . . . , n} representing the coordinates of the terrain, the point of highest importance p_{highest} is determined by:

$${p}_{highest} = {argmax}_{( {x}_{i} , {y}_{i} , {𝓏}_{i} ) ∈ P} {𝓏}_{i}$$

However, this point can also be any other location within the site area, depending on specific project requirements or other strategic considerations.

Once the point of highest importance is established, the next step involves calculating the threshold of acceptance for a given topography. This threshold is defined as the difference between the existing topography and a completely flat plane. A flat plane, in this context, is a geometric plane where all points have equal z coordinates, implying no elevation variation. The threshold is essentially a measure of the deviation of the current terrain from this ideal flat plane. Mathematically, this can be expressed as:

$$Threshold = {\sum }_{i = 1}^{n} \vert {𝓏}_{i} - {𝓏}_{flat} \vert$$

where zᵢ are the z coordinates of the terrain points and z_{flat} is the z coordinate of the flat plane, which could be the average elevation or another reference value.

To achieve the desired terrain flatness, we employ a minimization procedure analogous to that used in our flood minimization process. This involves adjusting the topography to minimize the threshold value, effectively reducing the overall terrain variation. The minimization can be formulated as an optimization problem:

$${min}_{{𝓏}_{flat}} = {\sum }_{i = 1}^{n} \vert {𝓏}_{i} - {𝓏}_{flat} \vert$$

This optimization problem may be solved using various numerical methods, such as gradient descent or other appropriate algorithms, depending on the complexity of the terrain and the specific requirements of the project.

In the context of achieving optimal terrain flatness, it becomes essential to consider not only the addition of material to low-lying areas but also the removal or redistribution of material from higher elevations. This dual approach, encompassing both peak shaving (removal of high spots) and valley filling (addition to lower areas), is collectively referred to as surface grading. Surface grading plays a critical role in all flattening processes, ensuring a balanced and efficient path to achieving the desired flatness.

Given the strategic importance of both additive and subtractive methods in terrain modification, our optimization framework must be adapted to accommodate the different weightings for these processes. The optimization problem can thus be reformulated to include terms that specifically account for the costs or impacts associated with material addition versus removal. This can be mathematically represented as

$${min}_{𝓏} ( {\sum }_{i} {w}_{add} {( {𝓏}_{i} - {𝓏}_{flat} )}^{2} + {\sum }_{j} {w}_{sub} {( {𝓏}_{j} - {𝓏}_{flat} )}^{2} )$$

where zᵢ and zⱼ are the z coordinates of the terrain points subject to addition (valley filling) and subtraction (peak shaving), respectively, z_{flat} is the z coordinate of the desired flat plane, w_{add} and w_{sub} are the weighting factors for addition and subtraction, reflecting the relative importance or cost of each process in the overall optimization. These weights allow for a nuanced approach to terrain flattening, recognizing that the effort or impact of adding material to low areas may differ from that of removing material from high spots.

This weighted optimization ensures that surface grading strategies are efficiently integrated into the terrain flattening process. By adjusting the weighting factors (w_{add} and w_{sub}), project managers can tailor the optimization to favor either additive or subtractive processes, depending on project-specific requirements, environmental considerations, or cost constraints.

Terraforming: terraforming process involves user-specified desired topography. Thus, little to no automation is used here, and the computational focus is placed on creating a user interface which enables lay people to easily specify both additive and subtractive topographical changes that they wish to occur. These will be accomplished through a series of purely additive injections, but also excavations and importation of surface fill material of various characteristics such as sand, biochar, wood chips and gravel among others.

### 1.4 Post-Processing of Ideal Topography

The system may incorporate post-processing steps to refine the outcomes and ensure their practical applicability. These steps include smoothing techniques, and feasibility assessments.

### 1.4.1 Smoothing & Filtering Approaches

Smoothing is a step that enhances the quality of the terrain model by reducing noise and irregularities. This can be particularly important in applications where the smoothness of the terrain is critical, such as in construction or landscape design. We employ two main techniques for smoothing:

Spline methods involve fitting a spline, a type of smooth polynomial function, to the data points. Splines are especially effective in creating a smooth and continuous surface over the terrain. The mathematical representation of a spline function, typically a cubic spline, is given by:

$$S ( x ) = {α}_{3} {x}^{3} + {α}_{2} {x}^{2} + {α}_{1} x + {α}_{0}$$

where a₃, a₂, a₁ and a₀ are coefficients that are determined based on the terrain data. The spline function ensures a smooth transition between data points, thereby generating a more naturally flowing terrain surface.

In certain embodiments, the system may use moving filters, which involve applying a filter over a window that moves across the data points. A common example is the moving average filter, where the value at each point is replaced with the average of neighboring points within the window. This technique helps in smoothing out short-term fluctuations and highlighting longer-term trends in the terrain data.

### 1.4.2 Terrain-Aware Convolutional Approach

In a more advanced embodiment, learned convolutional filters may be employed for specific types of terrain which are clustered based on topographical characteristics. This approach is efficacious due to its ability to mimic terrain using filters.

Convolutional filters are core components of Convolutional Neural Networks (CNNs) that perform convolution operations on input data, effectively capturing spatial hierarchies and features. A convolutional filter applies a weighted kernel to the input data, facilitating feature detection such as edges, textures, and patterns pertinent to terrain smoothing. Mathematically, the convolution operation for a two-dimensional input (e.g., a terrain elevation matrix) is defined as:

$$( f * g ) ( i , j ) = {\sum }_{m} {\sum }_{n} f ( m , n ) \cdot g ( i - m , j - n )$$

where ƒ represents the input terrain matrix, g is the convolutional filter (kernel), and (i, j) are the coordinates in the output feature map. This operation slides the filter across the input, computing dot products to produce a new matrix that highlights or suppresses specific features.

Training involves adjusting the convolutional filters' weights to minimize a loss function that quantifies the difference between the network's smoothed terrain output and the ground truth (or desired outcome). This process uses backpropagation and a gradient descent optimization algorithm to iteratively update the weights. Through training, the CNN learns filter weights that effectively reduce noise and irregularities specific to the terrain type it's trained on.

Before applying convolutional filters, terrain data is clustered into distinct types using unsupervised learning algorithms (e.g., K-means clustering on terrain features like slope, roughness, and elevation patterns). This step categorizes terrain into types (e.g., mountainous, flatlands, valleys) based on similarities in their features, which can be mathematically represented as:

$$Minimize {\sum }_{k = 1}^{K} {\sum }_{i ∈ {C}_{k}} {‖ {x}_{i} - {μ}_{k} ‖}^{2}$$

where K is the number of clusters, Cₖ is the set of points in cluster k, xᵢ is a feature vector representing the terrain, and μₖ is the centroid of cluster k.

For each terrain cluster, a dedicated CNN with convolutional filters is trained to smooth terrain data of that specific type. This specialization enables the network to learn the nuances and unique characteristics of each terrain type, applying the most effective smoothing techniques learned during training. The application phase involves identifying the cluster type of a new terrain dataset and then applying the corresponding CNN model with its specialized convolutional filters. This results in a terrain smoothing process that is highly adapted to the specific features of the terrain type, ensuring optimal noise reduction and feature preservation.

When applying the trained convolutional filters to a terrain cluster, the convolution operation is executed as described earlier, with each filter tailored to extract and smooth features relevant to the specific terrain type. The selection of the appropriate CNN model (and thus filters) based on the terrain cluster ensures that the smoothing process is highly effective, leveraging the specialized learning that occurred during training.

This approach unifies the analytical rigor of ML with the domain-specific nuances of terrain types, ensuring that each piece of terrain is processed in a manner that respects its inherent characteristics while achieving the desired smoothing effect. Through this method, the application of convolutional filters becomes not just a generalized solution but a suite of finely tuned tools, each designed for optimal performance on specific terrain landscapes.

### 1.4.3 Feasibility Assessments

Feasibility assessments are another useful post-processing step. These assessments evaluate whether the proposed terrain modifications are practical and implementable. Two main approaches are used for feasibility assessments:

Attempted injection simulations involve running simulations of subterranean injection processes to evaluate the statistical likelihood of successful implementation of the proposed topographies. These simulations use computational models to predict how the terrain would respond to injection processes, providing insights into the practicality and potential risks.

Curvature analysis focuses on the geometric aspect of the terrain. It involves analyzing the curvature of the terrain surface and comparing it to a maximal feasible curvature threshold. This threshold is determined either through ML algorithms trained on relevant data or through manual input based on expert knowledge. The analysis identifies coordinates on the surface mesh that exceed this threshold, indicating areas where modifications might be infeasible due to excessive curvature. The mathematical basis for curvature analysis involves calculating the curvature κ at each point, which can be represented as:

$$κ = \frac{\vert {f}^{″} ( x ) \vert}{{( 1 + {{f}^{′} ( x )}^{2} )}^{3 / 2}}$$

where ƒ(x) is the function representing the terrain surface, and ƒ′(x) and ƒ″(x) are the first and second derivatives, respectively.

### 1.5 Automated Elevation Site Planning Pipeline

For elevation purposes, the desired and actual geospatial meshes are then inputted into a pipeline to determine the optimal injection parameters for a site plan. In preferred embodiments, each injection location and respective parameters are chosen incrementally until the desired and simulated geospatial terrains are within threshold of each other.

### 1.5.1 Mesh Computation

In our pipeline for terrain modeling and modification, an initial step is the computation of the current surface mesh. This mesh is fundamental in representing the terrain's surface and serves as a baseline for subsequent modifications.

The computation of the current surface mesh varies depending on the iteration within the process. If it is not the first iteration, the current surface mesh includes modifications from previous iterations, such as those resulting from simulated injections. These injections represent changes to the terrain, such as additions or removals of material, which alter the surface topology. The mesh, in this case, is updated to reflect these changes.

If it is the first iteration, the current surface mesh is directly derived from the surface topology interpolation process. This process typically involves creating a digital representation of the terrain's surface based on collected data points. The surface topology interpolation might utilize methods like Delaunay triangulation or other suitable algorithms to create a mesh that accurately represents the terrain's surface contours.

Once the current surface mesh is established, the next step involves computing the difference matrix between the current and the desired topographies. This difference matrix is essential for understanding and quantifying the changes required to achieve the desired terrain configuration. The computation is straightforward and involves simple matrix subtraction, where each corresponding element of the current topography matrix is subtracted from the desired topography matrix. This matrix provides a point-wise representation of the differences in elevation between the current and desired surfaces.

In some embodiments of this process, iterative smoothing is applied to the difference matrix. This smoothing step is crucial as it increases the likelihood that the resulting difference matrix is manageable and realistic for implementation. Smoothing can be achieved through various techniques, such as Gaussian blurring or mean filtering, which help in reducing noise and abrupt changes in the matrix. The smoothing operation can be mathematically represented as:

$${S}_{ij} = \frac{1}{k} {\sum }_{m , n ∈ Neighborhood ( i , j )} {D}_{mn}$$

where Sᵢⱼ is the smoothed value at position (i, j) in the difference matrix, Dₘₙ are the values in the neighborhood of (i, j), and k is the number of elements in the neighborhood.

### 1.5.2 Injection Location & Parameter Computation

The optimal injection location (x, y) coordinate is then computed. In preferred embodiments, we assume that the optimal injection location for the current iteration is located at the maximal point of the difference matrix. In other embodiments, we learn this process into a model through iteratively choosing different locations and producing simulation predictions based on the resulting topology and difference matrices. In another embodiment, use FEA/CFD simulations to determine coefficients associated with all locations on the mesh grid which also considers the inter-aperture interactions.

Upon completing the initial stages, our system is designed to determine a variety of injection parameters. These include depth, drilling orientation, duration of injection, time-series of optimal slurry compositions (which can be either static, meaning consistent throughout the injection process, or dynamic, changing over time), duration of the initial fracking fluid, and the duration of the flushing fluid. To accurately calculate these parameters, the system employs computational methods, leveraging the information from the difference matrix and associated geospatial data.

In preferred embodiments, a singular ML-based model is employed. This model is trained to make inferences about all the mentioned characteristics, utilizing both the difference matrix and the geospatial data specific to the local neighborhood of the chosen coordinate. The difference matrix here plays a crucial role, quantitatively representing the deviation of current conditions from the optimal or desired conditions for each parameter. The model operates by analyzing this matrix alongside the geospatial data, applying a learning algorithm to discern the most effective injection parameters. The learning process of this model can be mathematically represented as an optimization problem:

$${min}_{θ} L ( θ ) = \frac{1}{N} {\sum }_{i = 1}^{N} {( {p}_{i} - f ( {x}_{i} ; θ ) )}^{2}$$

Here, L(θ) is the loss function, N is the number of training examples, pᵢ represents the actual values of the injection parameters, xᵢ is the combined input of difference matrix and geospatial data, ƒ denotes the ML model, and θ are the model parameters.

In alternative embodiments, the system employs ensemble model structures. These structures involve multiple models, each specializing in predicting different injection parameters. The ensemble approach may operate either sequentially or in series. Sequentially, the output from one model is used as additional input for the next, enhancing the accuracy of subsequent predictions. In series, each model functions independently, and their outputs are combined for a comprehensive understanding. Mathematically, the sequential process can be represented as:

$${p}_{n + 1} = {f}_{n} ( {p}_{n} , x ; {θ}_{n} )$$

$$for$$

$$n = 1 , 2 , … , N$$

Here, pₙ is the output of the nth model, ƒₙ represents the nth model in the sequence, x is the input feature vector including both the difference matrix and geospatial data, and θₙ are the parameters of the nth model.

Furthermore, in some embodiments, techniques such as directional drilling are disincentivized to promote more efficient methods. This is achieved by incorporating a cost term into the ML model's loss function, effectively penalizing the use of these techniques. Similarly, in numerical approaches, such methods are considered only after exploring other potentially efficacious alternatives. The cost term can be mathematically represented as an additional component in the loss function:

$$L ( θ ) = \frac{1}{N} {\sum }_{i = 1}^{N} {( {p}_{i} - f ( {x}_{i} ; θ ) )}^{2} + λ C ( {x}_{i} )$$

where C(xᵢ) is the cost function associated with less preferred techniques, and λ is a regularization parameter.

### 1.5.3 Injection Simulation

After finalizing the injection parameters, our system undertakes a critical phase of simulating the injection process. This simulation is used to predict and understand the potential impact of the injection on the local geospatial mesh—a digital representation of the area's terrain and subsurface characteristics. The approaches to this simulation vary and can be categorized into several distinct embodiments, each with its own set of methodologies and computational requirements.

In some embodiments, we opt for regenerating the entire geospatial mesh in conjunction with the simulation. This approach, while straightforward, can be computationally intensive as it involves a complete recreation of the mesh based on the simulated changes.

However, in preferred embodiments, we adopt a more nuanced approach where the simulation and mesh regeneration processes are distinct. This separation allows for more focused and efficient computational efforts, where the impact of the injection is simulated and then applied to the existing mesh.

The first embodiment of the simulation subsystems involves the use of ML-based methods. These methods are trained on historical real-world injection data, enabling them to accurately infer geospatial mutations from the injection parameters and existing mesh data. The training process for these models involves optimizing a predictive function that maps the relationship between injection parameters, existing mesh conditions, and the resultant geospatial changes. The predictive function could be represented as ƒ(parameters, mesh)=predicted mutation where the function ƒ encapsulates the learned relationship from the training data. These approaches can generally be classified as reinforcement and supervised learning-based.

The second embodiment involves Computational Fluid Dynamics (CFD) and Finite Element Analysis (FEA) simulations. These simulations offer a more detailed and physically accurate modeling of the injection process, but require significantly more processing power. CFD simulations analyze fluid flow and pressure distribution, while FEA is used to understand the structural response of the mesh to these forces. The mathematical basis for these simulations typically involves solving Navier-Stokes equations for fluid dynamics and elasticity or plasticity equations for structural behavior.

Finally, we consider numerical methods with simplifying assumptions about the behavior of the apertures and their interconnectedness. For instance, the shape of the aperture might be modeled as a radially (a)symmetric 3D ellipsoid, with dimensions influenced by injection depth and local geospatial characteristics. The mathematical representation of such an ellipsoid could be:

$$\frac{{x}^{2}}{{a}^{2}} + \frac{{y}^{2}}{{b}^{2}} + \frac{{z}^{2}}{{c}^{2}} = 1$$

where a, b, and c represent the ellipsoid's principal axes.

Other assumptions might include migratory behavior, such as surface pressure causing asymmetric slurry migration, and curling behavior, where local geospatial pressures increase the likelihood of vertical slurry movement during aperture widening. These behaviors can be quantified using differential equations that model fluid and solid mechanics.

### 1.5.4 Mesh Updating

In our preferred embodiments, a crucial subsequent step in the geospatial modeling process involves the integration of updated local mesh data into the global geospatial mesh. This integration is essential to ensure that local modifications are accurately reflected in the broader context of the global terrain model. This process is executed through two primary methods, each suited to different complexities of terrain data.

The first method is straightforward and is employed when the local modifications are relatively simple and align well with the existing global mesh structure. In this case, we reassign the vertical mesh values within the coordinate range of the local neighborhood to align with those of the updated local mesh. Mathematically, if M_{global} represents the global mesh and M_{local} the updated local mesh, for each point (x, y, z) in M_{local}, we update M_{global} as follows:

$${M}_{global} ( x , y ) ← {M}_{local} ( x , y )$$

This approach ensures that the changes made in the local mesh are directly transferred to the corresponding areas in the global mesh.

In more complex scenarios, particularly where there is a noticeable discontinuity between the edges of the local mesh and those contained within the original global mesh, a more nuanced rectification procedure is required. This discontinuity can manifest as abrupt changes in elevation or terrain features, which may not accurately reflect the natural topography. To address this, we consider several rectification strategies.

The first approach involves applying a smoothing algorithm over the boundary areas between the local and global meshes. The smoothing can be achieved through techniques like Gaussian blurring or averaging, which reduce sharp transitions and create a more gradual change in terrain features.

In this approach, we simulate additional terrain modifications (injections) in the transitional areas between the local and global meshes. These simulations aim to create a more seamless integration of the two meshes, effectively bridging the gap between them.

If the discontinuity is caused by the parameters of the original terrain modification, we may opt to adjust these parameters. This could involve altering the depth, extent, or other characteristics of the terrain injection to reduce or eliminate the observed discontinuity.

As a last resort, if the discontinuity cannot be rectified by other means, the original terrain modification (injection) may be excluded from the model. This decision is taken when maintaining the integrity of the global mesh is prioritized over the local modifications.

### 1.5.5 Mesh Comparison

Following the recomputation of the geospatial mesh, the current and desired geospatial meshes are compared. In alternative cases, only surface meshes are compared. In both cases, the difference between the current and desired meshes is compared to a threshold value. This comparison can be made using common error relations (MAE, MSE, etc.) or using the greatest point of difference (preferred embodiment). In particular embodiments, this threshold is tuned to optimize for the cost of injection, duration of injection, difficulty of injection (i.e. number of holes, use of directional drilling, etc.), thus allowing for site plans which will result with less precise topography but more optimal peripheral characteristics.

If the threshold of similarity is not met, then the cycle repeats—the difference matrix is recomputed with the newly attained geospatial mesh and desired geospatial mesh, optimal injection location is computed, and so forth. This cycle repeats until certain stopping criteria, depending on the embodiment, are met. In the simplest system, the cycle continues until the difference between the current and desired meshes is less than the predefined threshold. Additionally, to prevent indefinite operation, the system self-terminates after an arbitrary number of iterations if the condition is never met.

In the more complex version of our geospatial topology management process, we introduce a dropout condition to enhance both efficiency and feasibility, particularly relevant as the iterations of our terrain modification cycles progress. As the cycles advance, the likelihood diminishes that the previously chosen configuration of injection profiles will continue to yield a feasible and efficient site plan. To counteract this diminishing efficacy, we implement a strategy where previous injection profiles are either uniformly or in a biased manner removed or altered after a predetermined number of cycles. The decision to remove or alter these profiles is based on an evaluation of their contribution to achieving the desired terrain configuration. This evaluation can be quantified using a metric that assesses the effectiveness of each profile, which can be formulated as:

$$E ( {profile}_{i} ) = f ( currentstate , desiredstate , {profile}_{i} )$$

where E is the effectiveness metric, profileᵢ is the injection profile under consideration, and ƒ is a function that calculates the profile's contribution towards bridging the gap between the current and desired terrain states.

This adjustment may be useful in scenarios where persisting with the same injection profiles leads to diminishing returns or results in unfeasible terrain configurations. It ensures that the terrain modification process remains dynamic and responsive to changing conditions, optimizing the use of resources and time.

Another embodiment of our process involves dynamically modifying the threshold against which the difference between the current and the desired terrain is compared. This method is especially advantageous for sites where precise elevation control is less critical. We propose implementing this dynamic modification using piecewise defined methods or continuous adjustment profiles.

In the piecewise defined approach, the threshold changes at specific iteration intervals. To illustrate, we use an example of the threshold being set at 1 cm for the first 0-10 iterations, and then adjusted to 2 cm for the 10-20 iterations, and so on. This can be represented as:

$${Threshold}_{(} n ) = \{ \begin{matrix}1 cm & if 0 \leq n < 10 \\ 2 cm & if 10 \leq n < 20 \\ ⋮ & \end{matrix}$$

Alternatively, the threshold could be adjusted continuously over iterations following either an exponential or linear profile. For instance, in an exponential adjustment, the threshold at iteration n could be defined as:

$$Threshold ( n ) = a \cdot {e}^{bn}$$

where a and b are constants that define the rate of exponential change.

Moreover, this threshold adjustment could be governed by an optimizer approach, where the actual error between the current and desired geospatial meshes is factored into the threshold adjustment. This optimization-driven adjustment ensures that the threshold remains aligned with the practical requirements of the terrain modification process, adapting dynamically to the evolving error landscape.

$$Threshold ( n ) = Optimizer ( Error ( currentstate , desiredstate , n ) )$$

If the threshold of similarity is met, then the system outputs the predicted resulting geospatial topography, updated flood statistics, flatness coefficients, water pooling hotspot predictions, updated seismic data, and the associated site plan. The site plan can include but is not limited to drilling and injection guidelines. The drilling guidelines generally consist of locations, associated depths, and associated angle of directional drillings if used. The injection guidelines generally consist of aperture-associated information relating the duration of injection, multi-hole injection scheduling, duration of initial fracking fluid, duration of flushing fluid, and time-series of optimal slurry composition.

### 1.6 Automated Sequestration Site Planning Pipeline

For sites proposed for sequestration, the system does not optimize for elevation, but rather mass of carbon sequestered. Thus, we consider the additional user input of site characteristics, including information about pre-drilled holes and established apertures in addition to the previously described geospatial meshes. The system then proceeds to optimize the quantity that the given site can be used to sequester and the timeline in doing so.

In the preferred embodiment of our subsystem for carbon sequestration, we implement a simulation-based approach to optimize the injection of a specially formulated slurry into drilled holes. This method is designed to maximize carbon sequestration while ensuring the structural integrity and minimization of methane oxidation in anaerobic decay occurs at the injection sites.

The process begins by selecting an arbitrary hole, previously drilled, as the starting point for our simulation. The initial step involves injecting a baseline volume of slurry with a specific concentration optimized for carbon sequestration. This baseline slurry concentration is strategically chosen to maximize lignocellulose-rich components, known for their efficacy in sequestering carbon. The baseline volume of slurry is selected arbitrarily but is generally kept low to cautiously approach the optimal volume.

The simulation employs a method analogous to a ML optimizer, where the slurry composition and volume are iteratively adjusted to achieve the best sequestration outcome. This iterative process involves simulating the injection of increasing volumes of slurry into the aperture and closely monitoring the results. The slurry composition, comprising distinct additive proportions, is tuned in a manner similar to adjusting learning rates or weights in a ML algorithm. The mathematical representation of this tuning process can be likened to the optimization step in gradient descent:

$${V}_{n + 1} = {V}_{n} - α \frac{\partial L}{\partial {V}_{n}}$$

where Vₙ is the current volume of slurry, Vₙ₊₁ is the updated volume, α is a step size analogous to the learning rate, and

$$\frac{\partial L}{\partial {V}_{n}}$$

represents the gradient of the loss function L with respect to the volume. The loss function here is defined in terms of sequestration efficiency and potential injection issues.

This simulation continues until a point where issues related to the injection process, such as rupturing or clogging events, are predicted. Upon encountering such issues, the chosen volume is slightly reduced by a predetermined decrement. This decremented volume is then considered optimal for that particular aperture, ensuring maximum slurry injection without compromising the structural integrity of the hole. The aperture is subsequently marked as used.

The process is methodically repeated for each drilled hole until all are utilized, thereby ensuring that the site consists of apertures optimally filled for sequestration purposes. This systematic approach guarantees not only the maximization of carbon sequestration but also the maintenance of site integrity.

Upon completion of this process, the system generates predicted geospatial topographies and a detailed site plan. These outputs are designed to mirror the components typically found in site plans for elevation purposes, providing a comprehensive and realistic projection of the site post-injection.

### 1.7 Precision Elevation Methodologies

Throughout the process of site planning, particular focus is also placed on factors which enable and increase the ease with which precision elevation is achieved.

### 1.7.1 Modeling & Techniques

Accurate modeling and manipulation of the surface and subsurface hydrological character of the site is crucial. This involves predicting soil water movement to determine the ease of subterranean slurry injection, similar to horizontal hydraulic fracturing events, at different locations. This prediction can be achieved using CFD-based models which process soil porosity, permeability, and water retention characteristics and are essential for efficiently elevating the ground in targeted areas. The ability to control both surface and subsurface hydraulic conductivity is vital in this regard. Techniques such as intermediate smaller injections and land modification can be employed to achieve this control, ensuring a more uniform and controlled elevation process.

Another important aspect is the pre-saturation of the ground, which increases its ability to undergo hydraulic fracturing. By pre-moistening the soil, the risk of water leaking out dynamically during the injection process is reduced, allowing for a more controlled and effective elevation process. Additionally, the planting of trees and plants or the deposition of biochar can be utilized to increase the tensile strength of the ground's surface. This approach helps control the tensile dynamics in the ground, aiding in the mitigation of potential cracking. It is also important to note that if there are discontinuities in tensile strength between different adjacent areas, cracking might occur. Therefore, steps should be taken to either increase the tensile strength in specific areas or decrease it in others to prevent this issue.

Finally, the use of static or dynamic ground anchors can play a significant role in this process. These anchors increase the vertical tension exerted on the ground at different locations, guiding the shaping of the subterranean apertures. This technique enhances the precision with which the periphery of the injection aperture can be constructed, allowing for a more accurate and effective elevation process.

### 1.7.2 Strategic Property Damage

During the site planning stage, the concept of leveraging adjacent properties for the overall benefit of a project is an important consideration. This strategy might involve the tactical use of nearby land to enhance the characteristics of the primary property. For instance, sacrificing a plot of land adjacent to the one being elevated can improve drainage and overall land stability for the main site. Such decisions require a balance between the value of the land being sacrificed and the benefits gained for the primary property.

To facilitate more informed and comprehensive decision-making in land management and development, we propose a versatile computational model for the evaluation of sacrificial land. This model is adept at evaluating a broad spectrum of factors, such as property value, risk, potential benefits, and other relevant variables, thus allowing for an extensive cost-benefit analysis. The goal is to provide a multifaceted assessment framework, adaptable to various scenarios and capable of incorporating additional factors as needed.

The model systematically addresses several key aspects: At its core, the model analyzes property values, considering attributes like location, size, and amenities. The assessment encompasses both current and potential future values, factoring in market trends and potential developments. The value function, V(p, t, . . . ), includes variables such as property attributes p, time t, and other relevant factors like zoning laws or economic forecasts. The model conducts a thorough risk analysis, incorporating not just environmental and market risks, but also regulatory, socio-political, and technological risks, among others. The risk function, R(d . . . ), is designed to be expansive, where d represents development parameters and e encompasses a range of external factors. This allows for a comprehensive evaluation of the potential risks associated with any land-use decision. The benefits are assessed in a holistic manner, looking at economic gains, environmental impacts, social values, and more. The benefit function, B(s, u, . . . ), is structured to be inclusive, with s indicating the scope and u the utility, while also allowing for the integration of additional variables such as community impact or long-term sustainability.

For the cost-benefit analysis, we ensure a nuanced and thorough evaluation. The cost-benefit ratio CBR is just the starting point. We extend this by incorporating multi-criteria decision analysis (MCDA) and probabilistic risk assessment (PRA). MCDA allows us to weigh and aggregate diverse factors, while PRA introduces a probabilistic dimension to risk evaluation. The cost-benefit analysis can be represented as:

$$CBR = \frac{\sum {w}_{i} \cdot {B}_{i}}{\sum {υ}_{j} \cdot {C}_{j} + PRA ( riskfactors )}$$

where wᵢ and vⱼ are weights assigned to each benefit Bᵢ and cost Cⱼ respectively, based on their relative importance. The PRA(riskfactors) term introduces a probabilistic assessment of risks, adding depth to the traditional CBR calculation.

Additionally, the model can integrate advanced statistical models and ML algorithms to predict future trends and potential impacts, enhancing the predictive accuracy of the analysis. This could involve time series analysis, regression models, or even neural networks, depending on the complexity and nature of the data involved.

We further extend its application to evaluate not only the sacrifice but also the partial damages to properties, particularly in the context of implementing water management systems on peripheral properties. This extension allows for a nuanced analysis of the financial implications of ‘damaging’ a property, be it through physical alteration or the installation of water management systems like swales, bioswales, and drainage pipes. The model incorporates algorithms and methodologies to calculate the financial value of partial damages to properties. This involves:

Assessment of Property Modifications: The model evaluates the extent and nature of modifications made to a property, such as the installation of drainage systems or alterations in landscape. This is quantified in terms of its impact on the property's value, usability, and aesthetic appeal. The modification impact can be represented as a function M(d, ƒ, . . . ), where d represents the degree of modification and ƒ includes factors like functionality and visual impact.

Financial Valuation of Damages: A crucial part of the model is to compute the financial worth of these modifications or damages. This involves an intricate analysis of market trends, property valuation models, and potential future utility. The valuation is formulated as D(p m, . . . ), where p represents property attributes, and m is the extent of modification or damage. The model can also factor in indirect costs such as temporary loss of use or reduced attractiveness to potential buyers or renters.

Integration with Water Management Systems Analysis: In the context of water management systems, the model evaluates how these systems enhance or detract from the main property's value and functionality. The analysis includes calculating the cost of installation and maintenance of features like swales or drainage pipes against their effectiveness in improving drainage and reducing water-related risks. The benefit-cost ratio of these systems can be incorporated into the broader cost-benefit analysis of the entire property, using a function W(s, e, . . . ), where s represents the system specifications, and e, is the estimated effectiveness.

Building upon the earlier cost-benefit analysis framework, this extended model also factors in the costs associated with property damages, modification, and water management systems installation. The extended cost-benefit ratio now becomes:

$${CBR}_{extended} = \frac{\sum {w}_{i} \cdot {B}_{i} + W ( s , e , … )}{\sum {υ}_{j} \cdot {C}_{j} + D ( p , m , … ) + PRA ( riskfactors )}$$

In this equation, W(s, e, . . . ) represents the benefits derived from water management systems, while D(p, m . . . ), quantifies the financial impact of property damages or modifications.

Alternatively, we can use Real Options Analysis which introduces a financial perspective, particularly useful for evaluating investment decisions in property modifications under uncertainty. It treats investment decisions similar to financial call or put options, providing the right, but not the obligation, to undertake certain business initiatives, such as modifying a property or implementing water management systems.

The Black-Scholes model is a widely used method for valuing options in financial markets, which can be adapted for real estate investments. The value of a real option (V) to modify a property can be expressed as:

$$V = S \times N ( {d}_{1} ) - X \times {e}^{- rt} \times N ( {d}_{2} )$$

where S is the current value of the property improvements, X is the strike price, or cost of property modifications, N(d) is the cumulative distribution function of the standard normal distribution, and d₁ and d₂ are calculated using the formulae:

$${d}_{1} = \frac{ln ( S / X ) + ( r + {σ}^{2} / 2 ) \times t}{σ \times \sqrt{t}}$$

$$And$$

$${d}_{2} = {d}_{1} - σ \times \sqrt{t}$$

In these equations, r is the risk-free interest rate, σ is the volatility of the property value, and t is the time to expiration of the option.

This model provides a quantifiable method to assess the value of waiting or proceeding with property modifications, considering the time value of money and the uncertainty of future property values.

Furthermore, time series analysis is crucial for forecasting how property values might evolve over time, considering historical data and potential future trends. This can be particularly useful for long-term investment decisions in property modifications. AutoRegressive Integrated Moving Average (ARIMA) models, for example, are effective for forecasting non-stationary time series data, like property values, which can be influenced by various factors over time. An ARIMA model is generally denoted as ARIMA(p, d, q), where p is the number of autoregressive terms, d is the number of nonseasonal differences needed for stationarity, and q is the number of lagged forecast errors in the prediction equation. The ARIMA model, thus, can be represented as:

$${Y}_{t} = c + {ϕ}_{1} {Y}_{t - 1} + … + {ϕ}_{p} {Y}_{t - p} + {θ}_{1} {ε}_{t - 1} + … + {θ}_{q} {ε}_{t - q} + {ε}_{t}$$

where Yₜ is the property value at time t, c is a constant, ϕ are the parameters of the autoregressive terms, θ are the parameters of the moving average terms, and εₜ is white noise error terms.

### 2. Control System

The present control system is designed to enable the subterranean slurry injection system to function fully autonomously with minimal to no human intervention. It achieves this through the control of several different aspects of the injection procedure: aperture opening, clogging detection and handling, tank fill level control, and topology guidance to name a few.

### 2.1 Inputs & Outputs

The control system functions in real-time and thus listens and manages several data streams simultaneously or in batched fashion, which include but are not limited to GPS modules, pressure sensors (used either directly on piping or connected to capillaries which are inserted down-hole or also within piping), distance sensors, VFD outputs, flow meters, load cells (attached to pipes, placed under trailers/trucks, in-hole, etc.), strain gauges, surface strain gauges, leveling laser surveys, camera-tracked leveling stick movement, drone photogrammetry surveys, track-mounted camera photogrammetry surveys, lidar surveys, and/or resistivity surveys.

The system outputs instructions which control Variable Frequency Drives (VFDs), valves, ground anchor loads and flow rate to ensure precise management of fluid dynamics in various industrial processes.

Flow rate control can be implemented through several embodiments, each tailored to specific operational needs. These methods range from mechanical manipulations to pressure adjustments, each with its unique mechanism and mathematical principles.

One such method involves continuous positional control or open-closed configurations, which can be achieved using various types of valves and pumps. For instance, a pinch valve functions by pinching a flexible membrane to regulate the flow. The degree of pinching, and hence the flow rate, is controlled by the valve position, which can be mathematically represented as:

$$Flow rate = f ( Value position )$$

where ƒ represents the function defining this relationship, which can be empirically derived based on the characteristics of the valve.

Similarly, a knife valve uses a sharp edge to cut through the flow, and a single-stage progressive cavity pump utilizes a rotor-stator mechanism for flow control. These methods offer precise control over the flow, allowing for fine adjustments based on the system's requirements.

Another approach employs a section of rubber tubing inside the pipe, which inflates when pressurized, thus restricting the flow rate. The relationship between the pressure inside the tubing and the resulting flow rate can be expressed as:

$$Flow rate = g ( Internal pressure )$$

where g represents the function defining this relationship, which can be empirically derived based on the tubing's material properties and dimensions.

Additionally, the system can leverage a peristaltic pump or peristaltic pump-like mechanism, which uses a moving pinch to control the flow. This method is particularly effective in preventing clogging (plugging) and provides a static pinch when shut off, ensuring no flow leakage. The dynamics of flow control in this mechanism can be understood through the principles of peristaltic motion, which can be modeled and optimized for various fluid types and operational conditions.

A different class of approaches focuses on modifying the pressure inside the aperture itself. This alteration in pressure creates a back pressure, subsequently modifying the flow rate to the given hole. One way to achieve this is by using a dynamic ground anchor, which modifies the volume of the aperture, thereby influencing the flow rate. The relationship here can be quantified as:

$$Flow rate = h ( Aperture volume )$$

where h represents the function defining this relationship, depending on the physical characteristics of the aperture and the fluid properties.

Another method in this class involves injecting a pressurized air/mixture through the central or an auxiliary hole. This technique effectively alters the internal pressure and enables air expansion to reduce pressure drop with fluid removal or pressure rise with fluid placement and, consequently, the flow rate.

### 2.2 Control Methodologies

In order to accomplish the aforementioned control objectives, multiple methodologies are employed. These methodologies are treated computationally as functions, where learning techniques, manual input, or lookup tables are employed to attain a mapping and inverse mapping of the system inputs to outputs.

### 2.2.1 Slurry Composition

The first control method is to optimize the control of the slurry composition, our methodology encompasses dynamic and static approaches. The dynamic approach involves changing the composition over time, while the static approach maintains a constant composition throughout the injection process. This control is achieved through the precise deposition of material into a mix tank, which agitates the components to attain a mixture tending towards homogeneity. Alternatively, the control of slurry composition can be implemented through inline addition and/or mixing of additives. Additionally, the control of slurry composition can be implemented through in-hole addition and/or mixing of additives. In both of these embodiments, the materials are deposited using similar methods to the subsequent discussion regarding mix tank deposition.

The deposition of materials into the mix tank can be executed in various ways. In the preferred embodiment, we use separate pumps for the pumpable additives and conveyors for example belts or augers for the non-pumpable additives. The control of this mixture's composition is primarily executed through proportional control of the rates of the pump and the conveyor mechanism that deposits the non-pumpable additives. This is generally achieved using Variable Frequency Drive (VFD) control though mechanical drive rate systems are also possible.

For pumps, controlling the VFD is slightly more complex due to factors like differential pressure and the amperage drawn by the VFD. Differential pressure refers to the difference in pressure before and after the pump, represented as ΔP, and amperage, represented as A, is the electrical current drawn by the pump. The control relationship for the VFD can be mathematically represented as:

$$R = k \times f ( A , Δ P , Hz )$$

Here, R is the rate of deposition, k is a constant factor, ƒ is a function that outputs the RPM of the VFD based on amperage (A), differential pressure (ΔP), and frequency (Hz). This function ƒ encapsulates the complex relationship between these variables, ensuring that the pump operates efficiently while maintaining the desired slurry composition.

An essential aspect of this system is accounting for the delay period within the mix tank: the time elapsed from the deposition of materials to the point where they are adequately mixed and ready for pumping. This delay is critical for the overall process, as it impacts the timing and sequence of subsequent depositions. An alternative is to have inline mixing to avoid storage of premixed slurry and enable more nearly instantaneous shut down of solids addition.

To effectively manage this, the controls are designed to track both the volume and the rate of material deposition, modeling the slurry composition as a time-dependent sequence. This involves calculating the expected composition of the slurry over time, factoring in the specified additive ratios and the current composition within the mix tank. The model can be conceptualized as:

$$C ( t ) = {C}_{initial} + \sum ( Δ {C}_{additive} \times {R}_{additive} ( t - Δ t ) )$$

where C(t) is the composition of the slurry at time t, Cᵢₙᵢₜᵢₐₗ is the initial composition, ΔC_{additive} represents the change in composition due to each additive, R_{additive} is the rate of addition for each additive, and Δt accounts for the delay in mixing.

### 2.2.2 Aperture Jetting

The second methodology is the control of the jetting of the aperture. This process involves the use of pressurized water jets which are either a point-directed stream and rotated radially or are line-directed streams which are either held in place statically or rotated to jet out a disk-like shape inside the aperture. This procedure is generally a precursor to slurry injection or used to unclog in-hole faults or clean unneeded slurry from injection pipe. It may be controlled through VFD input attached to a water pump. To control this system, both the rate of jetting and the volume jetted may be tracked.

### 2.2.3 Motor Directionality

The third methodology is the control of motor reversal. This can be useful to unclog pipes of slurry and other mixtures and may be simply controlled using VFD input attached to pumps. This may also be done in order to evacuate water or mixture from apertures. To control this system, both the rate of pumping during motor reversal and the volume moved may be tracked.

### 2.2.4 Slurry Movement Control

The fourth methodology is the directional control of slurry. This method is generally used to move slurry from the outlet to different holes where we desire apertures to be formed and slurry to be deposited. This method uses the system outputs mentioned related to flow rate control. These system outputs may be used to proportionally or binarily control the rate of deposition of slurry into multiple apertures simultaneously, sequentially, or in batched fashion.

### 2.3 Control Goals

In our control system, we introduce an additional layer of computational abstraction—system goals—to enhance the capabilities of the real-time control system. This layer is designed to align with and execute the central goals of the system, which are detailed in a site plan and mentioned at the beginning of this section. The site plan acts as a blueprint, guiding the system's operations and methodologies.

### 2.3.1 Site Plan

In a preferred embodiment, the execution of the system's goals is outlined in the site plan, which can be either manually constructed or generated by the system itself. This plan includes detailed specifications for the use of various methodologies and system outputs. By specifying these elements in the site plan, we ensure adaptability and efficient distribution of the computational load across the system. The preferred embodiment emphasizes the utilization of site plan-specified system goals to optimize performance and response times.

These specifications within the site plan are typically saved and processed as either event- or time-series data. The event-based approach allows the system to progress to future states as specified in the site plan. This progression is facilitated by the identification of states similar to those outlined in the plan. For example, if the system's current topography closely resembles the topography at event step n in the site plan, the system can advance to event step m+1 and execute all associated system goals. The similarity between states is computed using different metrics depending on the nature of the state. For instance, topographical similarity might be calculated using distance metrics such as Euclidean or Manhattan distances:

$$d ( p , q ) = \sqrt{{\sum }_{i = 1}^{n} {( {p}_{i} - {q}_{i} )}^{2}} ( Euclidean distance )$$

$$d ( p , q ) = \sqrt{{\sum }_{i = 1}^{n} \vert {p}_{i} - {q}_{i} \vert} ( Manhattan distance )$$

where p and q are two points in Euclidean n-space, and pᵢ, qᵢ are the coordinates of these points. For sequestration levels, the computation might involve simple arithmetic differences.

Alternatively, the time-series approach represents a different methodology wherein the system follows a time-based sequence with specific goals and predicted system states linked to time stamps. This approach does not rely on state similarity computations but rather on a chronological sequence of actions. While this method is less preferred due to its rigidity compared to the event-based approach, it can be equally effective, especially when backed by accurate simulation modeling. The time-series data can be represented as a sequence of observations:

$$X = \{ {x}_{1} , {x}_{2} , … , {x}_{T} \}$$

where X is the time series, and xₜ is an observation at time t.

In the preferred embodiments focused on elevation-centric sites, our approach is to define the attended states by event-series topographic profiles as specified in the site plan. This leads to a crucial role for the real-time control system, which is to computationally determine the most effective approach to attain the specified topographic profile for the upcoming event in the series. This process involves a complex computational task where, at a given event t, the system must utilize predefined goals to transform the current ground state into the topographic profile outlined for the subsequent event t+1.

In sites where sequestration is the primary focus, the real-time control system's responsibilities might shift to prioritize different system goals over elevation. The system's adaptability allows it to target various objectives depending on the site-specific requirements.

### 2.3.2 Aperture Opening

In our approach to achieving the system goal of aperture opening, we employ a variety of methods, each tailored to effectively create openings in various geological contexts. The primary technique utilized is the jetting method, which is informed and guided by data streams from pressure sensors and Variable Frequency Drive (VFD) outputs. This multifaceted approach allows for adaptability and precision in the aperture opening process.

The preferred embodiment involves utilizing the jetting method until a drop in pressure is observed. This drop in pressure signifies the successful creation of an aperture. We characterize these drops by analyzing deviations from established data trends or filters. Mathematically, this can involve computing the difference between the observed pressure readings and a predefined baseline or expected pressure profile. The baseline can be established using statistical methods such as moving averages or more complex data fitting techniques, where the deviation can be represented as:

$$Δ P = {P}_{observed} - {P}_{expected}$$

where ΔP is the pressure deviation, P_{observed} is the real-time pressure reading, and P_{expected} is the pressure value predicted by the data fit or filter.

Alternatively, we can employ ML algorithms to understand the general behavior of newly drilled holes. These algorithms can be trained on historical data comprising geological composition and geospatial characterization of previous drilling operations. By doing so, the system learns to predict the optimal parameters for the jetting process tailored to specific geological conditions.

Another approach involves a more straightforward method of timing the jetting process. Here, the jetting is executed for a predetermined duration, after which it is automatically terminated. This approach is less data-driven and is based on average estimations of the time required to achieve aperture opening in various geological settings.

We also consider an approach where VFD output readings are closely monitored. In this method, specific triggers for jetting control are established based on the readings from the VFD, which is indicative of the equipment's operational status and efficiency. The triggers are predetermined thresholds in the VFD output that, once reached, signal the need to adjust or cease the jetting process.

Furthermore, a feedback loop can be established in the initial stages of the injection process. This loop functions by comparing the actual difficulty of injection, as evidenced by real-time data, against the expected difficulty. The magnitude of the jetting process is then controlled proportionally to this difference, allowing for dynamic adjustment based on the conditions encountered. Mathematically, this can be represented as:

$${J}_{magnitude} = f ( {D}_{actual} - {D}_{expected} )$$

where J_{magnitude} is the jetting magnitude, D_{actual} is the actual difficulty measured, D_{expected} is the expected difficulty, and ƒ is a function that determines the adjustment in jetting magnitude based on the difference in difficulties.

### 2.3.3 Clogging Detection & Handling

The second goal of the system addresses the detection and handling of in-hole and in-pipe clogs. This is achieved through a combination of sophisticated sensor technologies and control strategies. Clogs are detected using an array of sensors, including flow meters, pressure sensors, distance sensors, and variable frequency drive (VFD) feedback output. The simplest method of detection involves identifying outlier data from these sensors through statistical fits, learned discriminants, or filters. Anomalous readings that deviate significantly from expected patterns can indicate potential clogging events. Mathematically, this could involve applying statistical tests or anomaly detection algorithms to sensor data to identify outliers.

Alternatively, we employ supervised learning to establish a predictive framework for clog detection. This approach involves training models on trends in sensor data to assess the likelihood of clogging. The training involves optimizing a model based on historical data (either simulated or observed), where the model learns to correlate specific sensor data patterns with clogging events. The mathematical formulation for this supervised learning could involve regression or classification algorithms, depending on the nature of the sensor data.

For handling clogging events, we utilize a combination of motor reversal and water jetting strategies. In-hole clogs, typically occurring in the vicinity of the hole, are addressed by water jetting. In contrast, in-pipe clogs are more effectively handled through motor reversal and fluid injection at the aperture pipe via the jetting system. The control system employs a mapping from the system inputs to these interventions, which can be established through learned models or bang-bang hysteresis controls.

In the case of learned models, we use supervised learning, possibly augmented by CFD and/or FEA simulations, to develop models that guide the response to clogging. These models can also be reinforced by a reward-based system, where the reward is configured based on sensor values indicating unclogged states.

Bang-bang hysteresis control, another effective strategy, operates on specified thresholds of sensor readings. Different thresholds correspond to unclogged or clogged states, triggering appropriate responses like water jetting or motor reversal. These responses can be implemented using either dynamic method profiles, which adapt based on real-time data, or static profiles, which operate on predefined rules.

### 2.3.4 Tank Fill Level

The third key objective of our system is the precise control of the fill level within the mix and/or storage tank. This control is crucial for preventing operational issues such as overflows or air suction through the outlet. The system employs a variety of approaches to achieve this, focusing on different parameters like volumetric additive proportions. Although volumetric measurements are typically used, other factors like mass, carbon content, density, and additional characteristics can also be considered.

In most cases, the system operator specifies the desired additive proportions based on the requirements of the task at hand. However, this process can also be automated. It's important to note that the mix tank is actively being pumped into the hole, necessitating consideration of the outlet pumping rate in maintaining the appropriate fill level.

The system relies on input from various sensors to monitor and control the fill level. These sensors include distance sensors, VFD outputs, impedance sensors or other level-sensing devices, load cells, and pressure sensors. Each sensor type provides critical data for accurately assessing the mix tank's status.

To compute the current fill level, we may employ different methods. One approach is to use a learned regressor. Here, the different sensor readings are measured at various controlled fill levels. This data is then utilized in a supervised learning framework, training a model to predict the current mix tank fill level accurately. The model is developed by correlating sensor readings with known fill levels, refining its predictions through techniques like regression analysis.

Alternatively, a direct method involves using a linear combination of the sensor readings to infer the mix tank fill level. This approach might use a formula like:

$$Fill Level = a \cdot {x}_{distance} + b \cdot {x}_{VFD} + c \cdot {x}_{impedance} + …$$

where x_{distance}, x_{VFD}, x_{impedance}, . . . are the readings from the respective sensors, and a, b, c, . . . are coefficients determined through calibration.

### 2.3.4.1 Tank Fill Level Embodiment 1

For controlling the fill level to the desired state, the system may utilize one of several strategies. The first is a bang-bang hysteresis control, which uses predefined thresholds around the desired fill level. This method employs a simple control mechanism based on the logic:

If the fill level exceeds Lᵤₚₚₑᵣ=L_{desired}+ΔL, reduce the rate of additive deposition.

If the fill level falls below L_{lower}=L_{desired}−ΔL, increase the rate of additive deposition.

Maintain the current rate if the fill level is within these thresholds.

### 2.3.4.2 Tank Fill Level Embodiment 2

Another strategy is a continuous scaling scheme, which continuously adjusts the rate of additive deposition based on the real-time difference between the actual fill level (L_{actual}) and the desired fill level (L_{desired}):

$$Adjustment = {K}_{p} \times ( {L}_{desired} - {L}_{actual} )$$

### 2.3.4.3 Tank Fill Level Embodiment 3

The optimizer scheme for controlling the fill level within the mix tank integrates principles from optimization algorithms commonly found in ML, specifically focusing on iterative refinement to achieve the desired fill level with high precision. This approach involves continuously adjusting the material deposition rate based on feedback from the system's current state, akin to how an algorithm like Stochastic Gradient Descent (SGD) or Adaptive Moment Estimation (ADAM) refines model parameters.

In the context of our system, the optimizer algorithm functions by first establishing a target fill level, F_{target}, which represents the ideal state for the mix tank's content. The system then calculates the current fill level, F_{current}, utilizing input from the various sensors as previously described. The difference between F_{current} and F_{target}, denoted as the error term E=F_{target}−F_{current}, guides the adjustment process.

To systematically adjust the material deposition rate, the algorithm follows a structured mathematical approach:

The system may first compute the gradient between current and desired fill levels:

$$∇ E = \frac{\partial E}{\partial V} = \frac{\partial ( {F}_{target} - {F}_{current} )}{\partial V}$$

where ∇E represents the gradient of the error with respect to the control variable V (e.g., rate of additive deposition or pump speed).

In an SGD-like approach, the update rule can be expressed as:

$${V}_{new} = {V}_{old} - α \cdot ∇ E$$

where V_{new} and V_{old} are the new and old values of the control variable, respectively, and α is the learning rate, a parameter that determines the size of the step taken towards minimizing the error. For an ADAM-like approach, which incorporates both momentum and adaptive learning rates, the update rule becomes more complex, incorporating moments of the gradients:

$${V}_{new} = {V}_{old} - \frac{α \cdot {m}_{t}}{\sqrt{{υ}_{t}} + ϵ}$$

where mₜ and vₜ represent the first and second moments of the gradients, respectively, and ϵ is a small number to prevent division by zero.

To ensure operational safety and effectiveness, the updates to V are constrained within predefined bounds:

$${V}_{new} = min ( max ( {V}_{new} , {V}_{min} ) , {V}_{max} )$$

where Vₘᵢₙ and Vₘₐₓ are the minimum and maximum allowable values for the control variable.

This process repeats iteratively, with F_{current} being recalculated after each adjustment to V, until the fill level stabilizes around F_{target} within an acceptable margin of error.

This mathematical framework enables precise, adaptive control of the fill level in the mix tank, leveraging the error between the current and target fill levels to iteratively refine the material deposition rate. Through this optimization process, the system dynamically responds to changes, ensuring optimal operation and preventing issues like overflows or insufficient mixing.

### 2.3.5 Topography

The final system goal for elevation-focused sites is topography control—that is, strategically manipulating various system methods to achieve predetermined topographic states as specified in the site plan or by the user. This process occurs at specific times or in response to particular events. To realize this objective, the system comprehensively utilizes all available inputs and exercises precise control over slurry composition, directional routing, and the general speed control of VFDs.

The approach begins with a comparison between the desired topographic state, as defined in the site plan or by the user, and the current state derived from various system inputs. These inputs initially base their readings on the topography outlined in surveys or other similar site characterizations. The system then iteratively conducts topographic simulations using these inputs, mirroring the procedures outlined in the site planning specifications.

These simulations play a crucial role in the system's decision-making process. They are executed for various candidate system states, with each simulation measuring the difference between the resulting simulated topography and the desired topography. Mathematically, this can be expressed as an optimization problem, where the objective is to minimize the difference (or error) between these two states. This error can be quantified using a suitable metric, such as mean squared error (MSE) or a related statistic.

After a determined number of simulations, the system selects the system state configuration that yields the minimum difference, thereby identifying an optimal set of system states. This process ensures that the topography is adjusted in a way that closely aligns with the predetermined specifications, thus achieving precise topographic control.

Alternatively, the system may employ a learned relationship to determine optimal system states. This relationship, established through supervised learning, is based on historical data correlating system states with resulting terrain configurations. The model, trained on simulated or real observed injection and topography data, learns to predict the terrain outcome based on different system states. This predictive model can be represented as a function:

$${Terrain}_{predicted} = f ( System States ; θ )$$

where ƒ is the model, the system states are the inputs, and θ represents the learned parameters of the model. By applying this learned relationship, the system can efficiently predict and select system states that will most likely result in the desired terrain configuration.

In our systematic approach to managing system goals, we implement an algorithm that aims to achieve an optimal geospatial mesh configuration at each iterative step. This algorithm, adaptable to various methodologies, assesses and implements the most feasible system state, ensuring alignment with the desired topography and system functionalities.

The process commences at a designated time or event step, where the system extracts the optimal geospatial mesh from the site plan or via manual input for the next time step. This involves computational geometry techniques to transform the current terrain data into a detailed mesh grid representation, effectively mapping the terrain's existing features.

Subsequently, the system consults the topographic control system goal subsystem. This subsystem generates a list of potential optimal system states that are likely to achieve a topography similar to the target configuration in the upcoming time step. This task might necessitate the integration of various system goals, each contributing to the overall topographic outcome. The optimal states are identified through a multi-objective optimization problem, balancing diverse parameters to pinpoint the most suitable state.

In parallel, the system conducts independent queries for other goals, addressing specific concerns like clog detection and fill level control. This comprehensive approach ensures that all relevant factors influencing the system's performance are considered.

### 2.3.6 Feasibility Assessment

Upon identifying the most optimal system state, its feasibility is rigorously evaluated. This evaluation encompasses two main methods: constraint satisfaction and simulation testing. Constraint satisfaction checks if the proposed state complies with the system's predefined constraints, including physical limitations and operational parameters. This can be mathematically formulated as a series of inequalities:

$${g}_{i} ( x ) \leq 0 , {h}_{j} ( x ) = 0$$

where gᵢ(x) are the inequality constraints and hⱼ(x) are the equality constraints, with x representing the system state variables.

Simulation testing involves running the proposed state through a virtual model to predict its performance and identify potential undesirable behaviors. This step is pivotal for visualizing and understanding the implications of the state before actual implementation.

An essential component of the feasibility assessment is the incorporation of query learning, particularly in scenarios requiring human intervention for real-time evaluation. Query learning is a ML technique where the model actively queries a human expert to provide labels or guidance on uncertain data points. The mathematical basis for query learning can be represented through a Bayesian framework, where the model updates its beliefs based on the expert's feedback. This can be expressed as:

$$P ( θ \vert D , Q ) ∝ P ( D \vert θ , Q ) \cdot P ( θ )$$

where P(θ|D, Q) is the posterior probability of the model parameters θ given the data D and query Q, P(D|θ, Q) is the likelihood of the data given the model parameters and query, and P(θ) is the prior probability of the model parameters. This approach continuously refines the model's accuracy in feasibility assessments.

If a candidate system state is determined to be unfeasible, it is discarded, and the next most optimal state undergoes evaluation. This iterative process is repeated until a feasible state is identified. Once a state is accepted, the system simulates the resulting changes to the geospatial mesh using the same simulation system as the site plan. The simulation assesses whether the modified mesh aligns with the desired configuration within a specified threshold.

If the simulated state meets the criteria, it is approved for implementation. The system then executes the specified state for the current time or event step and repeats the process for subsequent steps, ensuring consistent alignment with project goals and operational parameters.

### 2.3.7 Alert Mechanisms

In the system designed to minimize the occurrence of undesirable events such as clogging, aperture rupturing, aperture drift, and general machine malfunction, we have incorporated a sophisticated human alert mechanism. This mechanism plays a crucial role in ensuring the safety and efficiency of the operation by promptly notifying the operator of any issues and suggesting potential rectification strategies.

The alert mechanisms are primarily audiovisual indicators, designed to provide clear and immediate information about the nature of the event. These indicators not only specify the type of event occurring but also offer guidance on possible corrective actions. The visual component could be in the form of on-screen messages or lights, while the audio component may include alarms or spoken instructions.

The system may use ML algorithms that analyze data from various sensors integrated into the system. These models are trained to recognize patterns indicative of potential issues, such as unusual vibrations or temperature changes, which could signal the onset of malfunctions like clogging or aperture drift.

The system may also use warnings which are generated based on parameters set either manually or through a site plan input. They act as thresholds or benchmarks against which the current system state is continuously compared. For instance, if the system state deviates from the predefined safe operating conditions, an alert is triggered.

Alternatively, in sensor outlier analysis, statistical methods are used to analyze sensor data for outliers—readings that deviate significantly from the norm. Such deviations may indicate potential issues within the system. The analysis could involve techniques like standard deviation calculations or more complex anomaly detection algorithms.

Furthermore, a preferred embodiment of our system includes a proactive alert feature. This feature notifies the operator of significant discrepancies between predicted and observed topographical or system behaviors over time or between event steps. For example, if the actual terrain modification differs substantially from the predicted outcome, an alert is raised. This proactive approach serves as a surrogate for specific event detection, allowing for early intervention before issues escalate.

Upon detection of an event, the system is programmed to execute a safety procedure alongside the operator alert. This procedure is primarily passive, designed to safeguard property, equipment, and personnel. It could involve actions like equipment shutdown or a controlled wind-down of operations. The nature of this safety procedure may be predefined, learned from past experiences, or determined through simulation-based scenarios.

Additionally, our system engages in a continuous improvement loop. Post-event, it queries the operator for specific procedures that successfully rectified the error. This feedback is then integrated into a continual learning query oracle system, which enhances the system's capabilities. This oracle system not only broadens the range of system goals but also refines the approaches used within these goals, thereby improving the overall reliability and safety of the operation.

### 2.3.7 Additive Supply

In addition to execution of injection procedures, the control system may assist the operators in the procurement and/or delivery planning and/or execution of slurry components. Through the use of site plans and/or manual entry, the system may compute the duration of time that the injection can be executed using the current supply of slurry components. Thus, the system may automatically plan and/or execute the procurement of additional components, thus replenishing the site's available stock.

### 3. Self-Learning Cycle

Throughout the previous system descriptions, two main models have been referred to: a model which simulates injections (i.e. takes injection parameters and geospatial meshes as input and predicted post-injection geospatial meshes) and a model which infers optimal injection parameters (i.e. takes current and desired geospatial meshes as input and outputs optimal injection parameters to translate the terrain between the inputted states). In addition to all other mentioned models, these are subject to refinement, namely fine-tuning, if learning models are used.

In the subsequent description of this self-learning cycle, we refer to the models as purely learning models, although in reality, the models are a combination of deterministic simulation, probabilistic learning-based, and user input driven models.

### 3.1 Injection Parameter Model

Throughout the use of the injection parameter model, inferences are made (in the site planning and control system) in order to infer optimal parameters to achieve specific geospatial meshes. This cyclic querying of the model produces respective candidate injection parameter sets based on current meshes (geospatial mesh 1) and desired resulting meshes (geospatial mesh 2). The output of these inferences may then be subsequently used as input into the injection simulation model and inferences are outputted as resulting geospatial meshes. Alternatively, the candidate parameter set may be executed as a system state in the injection machine and sensor readings used to derive a resulting geospatial mesh. Regardless of the embodiment, the difference between the resulting and geospatial mesh 2 are computed and, if within tolerance, the candidate parameter set is then outputted. If the difference is outside the tolerance, then the difference matrix is computed. This difference matrix then undergoes an error calculation. This calculation may be different depending on the embodiment, but is generally considered as a representation of error. The calculation may be based on maximal elements in the difference matrix, an average error (MSE, MAE, etc.), a learned relation, a distance relation, etc. This representation of error is then used to compute a cost function used in the refinement of the injection parameter model. The subsystem used in the refinement depends on the specific embodiment. In preferred embodiments, the injection parameter model is fine-tuned using a reinforcement learning cycle. This cycle uses the cost function mentioned above to penalize poor parameter inferences and encourage more accurate outputs. Alternatively, the system can utilize a supervised learning approach where the output of the simulation system or real-world geospatial mesh observations are used to label training data (i.e. injection parameters). Throughout the process of model refinement, a specific proportion of the data and labels are withheld for model evaluation, enabling continuous tracking of model performance. Additionally, clustering or other unsupervised approaches can be used to determine the general tendency of poor performing data sets and thus enable manual or automatic detection of geospatial meshes that tend to lend poor inferences. This may enable querying of manual input to infer optimal injection parameters more accurately in edge cases and alert operators to when the model is least performant. Additionally, confidence scores may be outputted to the operator to better inform manual intervention.

### 3.2 Injection Simulation Model

To refine the injection simulation model, primarily real-world or more accurate (although likely more computationally costly) simulation models are used. The more accurate simulation models may be based on manually or automatically prepared and executed FEA/CFD simulations. Real-world data is collected using sensor data from the aforementioned sensor array. In both cases, data is constructed through associating injection parameter sets, geospatial meshes, and sensor data into a time- or event-series. Through the injection simulation inferences made using injection parameter inputs, resulting data is generated continually. The time- or event-series is then segmented either into temporally- or event-coherent batches where data (injection parameter sets and/or sensor data) is associated with labels (geospatial meshes). This dataset can be interpreted as system states being the data and the geospatial meshes resulting from the execution of the system states as being the labels. Thus, we may split the dataset into train and test sets using a certain static or dynamic proportion. Using the train set, we perform refinement on the injection simulation model and additionally evaluate the performance of the model using the running test set (which is concatenated with the newly procured split dataset).

### 3.3 Learning Methodologies

In the injection models, we implement a Reinforcement Learning (RL) framework, tailored to predict optimal injection parameters and simulation schemes through an iterative process of trial and error. This RL framework operates on the principle of action-reward correlation: the model, acting as an agent, selects actions (parameter predictions) and receives feedback in the form of rewards. The reward function is critical here; it is designed to assign a numerical value to each action based on its outcome, where higher rewards are given for actions leading to outcomes closer to the desired objective. The mathematical formulation of this reward function is crucial and should be designed to reflect the specific goals of the injection process.

In refining the decision-making policy of the model, we employ policy optimization techniques such as Proximal Policy Optimization (PPO) and Deep Deterministic Policy Gradients (DDPG). PPO works by adjusting the policy in small, controlled steps, using a clipped objective function to prevent disruptive updates, which can be represented as:

$$L ( θ ) = \hat{𝔼} [ min ( {r}_{t} ( θ ) {\hat{A}}_{t} , clip ( {r}_{t} ( θ ) , 1 - ϵ , 1 + ϵ ) {\hat{A}}_{t} ) ]$$

where L is the objective function, θ represents the policy parameters, rₜ(θ) is the probability ratio, Âₜ is an estimator of the advantage function at time t, and ϵ is a hyperparameter defining the clipping range. DDPG, on the other hand, combines Q-learning and policy gradients, where the Q-function is learned using the Bellman equation, and the policy is updated using the gradient of the Q-function.

Within this RL framework, Q-learning can serve as a useful algorithm, enabling our model to ascertain the optimal action-selection policy without requiring a model of the environment. Q-learning is a value-based method that iterates over actions to estimate the “quality” or Q-value of state-action pairs, guiding the agent towards the highest reward. The essence of Q-learning lies in its iterative update equation, which refines Q-values based on the observed rewards and the maximal future rewards, encapsulated by the formula:

$$Q ( s , a ) ← Q ( s , a ) + α [ r + γ {max}_{{a}^{′}} Q ( {s}^{′} , {a}^{′} ) - Q ( s , a ) ]$$

where s and s′ represent the current and next state, a and a′ are the current and next actions, r is the immediate reward, α is the learning rate, and γ is the discount factor that weighs the importance of future rewards. This self-updating mechanism ensures that the model progressively converges towards an optimal policy that maximizes the cumulative reward, making Q-learning an integral part of enhancing our RL framework's decision-making capabilities.

A supervised learning approach with a feedback loop is also integrated. In this approach, the model is initially trained on a labeled dataset (real-world or simulated), and the learning process is continually refined based on the performance feedback. This process involves adjusting the model parameters to minimize a loss function, often using gradient descent methods. Advanced optimizers like Adam or RMSprop are employed here for efficient convergence. Adam optimizer, for instance, computes adaptive learning rates for each parameter and can be represented as:

$${θ}_{t + 1} = {θ}_{t} - \frac{η}{\sqrt{{\hat{υ}}_{t}} + ϵ} {\hat{m}}_{t}$$

where θₜ are the parameters at time t, η is the learning rate, \hat{m}ₜ and \hat{v}ₜ are bias-corrected estimates of first and second moments of the gradients, and ϵ is a small scalar used to prevent division by zero.

To effectively address imbalances in the training data, our approach incorporates advanced data augmentation techniques, specifically Synthetic Minority Over-sampling Technique (SMOTE) and Adaptive Synthetic Sampling Approach (ADASYN). These methods are critical for enhancing the model's predictive accuracy and generalization capabilities, particularly when dealing with skewed datasets where certain classes of data are underrepresented.

SMOTE operates by creating synthetic samples from the minority class instead of creating copies. This is achieved by randomly selecting a point from the minority class and computing the k-nearest neighbors for this point. The synthetic points are then created by choosing one of the k-nearest neighbors and forming a linear combination of the feature space. Mathematically, for a minority class sample xᵢ, a new sample x_{new} is generated as follows:

$${x}_{new} = {x}_{i} + λ \times ( {x}_{zi} - {x}_{i} )$$

where x_{zi} is one of the k-nearest neighbors of xᵢ and λ is a random number between 0 and 1. This process results in a more diverse and representative dataset, which is crucial for training robust models.

ADASYN, similar in spirit to SMOTE, takes an additional step by adapting the number of synthetic samples generated for each minority class sample, based on the density of the class. It calculates the number of synthetic samples to create for each minority class sample by considering its k-nearest neighbors and how many of these neighbors belong to the majority class. This results in more synthetic samples being created for minority class samples that are surrounded by majority class samples, thus focusing on the harder-to-learn examples.

The system may additionally employ meta-learning; this approach is employed to enable the model to quickly adapt to new, but related tasks, a feature particularly useful in dynamic environments. In meta-learning, the model is trained on a variety of learning tasks, with the aim of learning an underlying structure or patterns common across these tasks. The model effectively learns to learn. Mathematically, this involves optimizing for a model parameter θ that can quickly adapt to a new task with only a small number of gradient steps. This can be formulated as:

$${θ}^{′} = θ - α {∇}_{θ} {L}_{{T}_{i}} ( {f}_{θ} )$$

where θ′ is the updated model parameter after training on task Tᵢ, α is the learning rate, L_{T}_{ᵢ}, is the loss on task Tᵢ, and ƒ_{θ} is the model parameterized by θ. The key here is to find a parameter space where the model has maximal adaptability to new tasks with minimal additional training.

The injection simulation model significantly benefits from the implementation of transfer learning and fine-tuning techniques. Transfer learning involves leveraging pre-trained models that have been previously trained on related tasks—such as Finite Element Analysis (FEA) or Computational Fluid Dynamics (CFD) simulations—and applying them to the injection simulation task. This approach capitalizes on the knowledge these models have already acquired, thereby reducing the need for extensive training from scratch. Mathematically, this involves taking a model ƒ(x; θₚᵣₑ), which is trained on a related task with parameters pre, and adapting it to the new task. The adaptation is usually done by modifying some of the final layers of the model and retraining these layers with the injection simulation data.

Fine-tuning complements transfer learning. Here, the pre-trained model is further refined to make it more suitable for the specific nuances of the injection simulation task. This involves adjusting the model parameters θ using the injection simulation data, which can be represented as an optimization problem:

$${θ}^{*} = arg {min}_{θ} L ( y , f ( x ; θ ) )$$

where L is the loss function, y is the true value, and ƒ(x; θ) is the prediction of the model. Fine-tuning helps in improving the accuracy and specificity of the model for the injection simulation task.

Active learning is another critical strategy employed in this model. In this approach, the model identifies and queries for labels (e.g., geospatial meshes) for the most informative data points during the training process. This is particularly effective in scenarios where labeling data is expensive or time-consuming. The model is designed to select data points for which it is least certain about the correct output, optimizing the learning process. The selection criterion can be based on uncertainty measures such as entropy, margin, or least confidence.

Federated learning is incorporated to manage data distributed across multiple injection sites. This decentralized approach enables each site to train a local model on its data. The local models are then aggregated to form a global model. The key mathematical concept here involves updating the global model G using the weighted average of the parameters of local models Lᵢ:

$$G = {\sum }_{i = 1}^{N} {w}_{i} {L}_{i}$$

where wᵢ is the weight assigned to the local model Lᵢ based on factors like the size of the dataset at each site. This approach ensures privacy and security, as individual data points do not leave their respective sites, yet allows for the collective intelligence of all sites to be harnessed.

Elastic Weight Consolidation (EWC) is a pivotal method for overcoming catastrophic forgetting, a common issue in neural networks where learning new tasks can lead to a loss of performance on previously learned tasks. EWC addresses this by selectively slowing down the learning on certain weights in the neural network, based on their importance to previous tasks. This importance is quantified using a measure known as the Fisher Information Matrix (FIM). Mathematically, the loss function in EWC is augmented to include a term that penalizes changes to important weights. This can be represented as:

$$L ( θ ) = {L}_{new} ( θ ) + {\sum }_{i} λ {{F}_{i} ( {θ}_{i} - {θ}_{i , old} )}^{2}$$

where L_{new}(θ) is the loss for the new task, θᵢ are the parameters of the model, θ_{i,old} are the parameters from the previous task, Fᵢ is the Fisher Information for each parameter, and λ is a hyperparameter that controls the strength of the penalty.

Experience Replay is another method employed to enhance the stability and retention of knowledge in the model. It involves maintaining a memory buffer of past experiences (data points or episodes). The model is periodically trained on a mixture of new and old data, allowing it to ‘rehearse’ and retain previous knowledge. This process can be represented as retraining the model on a dataset D comprising a combination of new data D_{new} and a randomly sampled subset of past data Dₚₐₛₜ from the memory buffer.

Curriculum Learning is implemented to facilitate more stable and gradual adaptation of the model to complex tasks. In this approach, the model is initially exposed to simpler or more basic tasks or data, gradually increasing in difficulty or complexity. This progression can be mathematically modeled by defining a series of tasks T₁, T₂, . . . , Tₙ with increasing difficulty and adjusting the training regimen to move from T₁ to Tₙ. The progression through these tasks can be controlled by a curriculum function C(t) that decides the task to present at each training step t.

The implementation of advanced optimizers, namely AdaGrad, Adam, and BFGS (Broyden-Fletcher-Goldfarb-Shanno algorithm), plays a central role in the training process by dynamically adapting the learning rate. These optimizers are essential for efficiently navigating the parameter space to minimize the loss function.

AdaGrad adjusts the learning rate for each parameter based on the historical gradient information. It accumulates the square of the gradients in a term Gₜ (a diagonal matrix where each diagonal element is the sum of the squares of the gradients w.r.t. each parameter up to time step t). The parameter update rule is:

$${θ}_{t + 1} = {θ}_{t} - \frac{η}{\sqrt{{G}_{t}} + ϵ} ⊙ {g}_{t}$$

where θₜ is the parameter vector at time t, η is the initial learning rate, ϵ is a small smoothing term to avoid division by zero, and gₜ is the gradient at time t.

Adam combines the benefits of AdaGrad and RMSprop. It maintains two moving averages for each parameter—one for the gradients (like RMSprop) and one for the square of the gradients (like AdaGrad). The parameter update rule is given by:

$${θ}_{t + 1} = {θ}_{t} - \frac{η}{\sqrt{{\hat{υ}}_{t}} + ϵ} {\hat{m}}_{t}$$

where \hat{m}ₜ and \hat{v}ₜ at are bias-corrected estimates of the first and second moment of the gradients, respectively.

BFGS is an iterative method for solving unconstrained nonlinear optimization problems. It belongs to quasi-Newton methods and approximates the inverse of the Hessian matrix of the second-order partial derivatives of the function. The update rule involves using this approximation to compute the direction of the parameter update.

Normalization techniques, specifically Batch Normalization and Layer Normalization, are incorporated to ensure stable and faster convergence of the model. Batch normalization normalizes the input of each layer for each mini-batch to have a mean of zero and a variance of one. Mathematically, it transforms an input X to:

$$\hat{X} = \frac{X - {μ}_{B}}{\sqrt{{σ}_{B}^{2} + ϵ}}$$

where μ_{B} and σ_{B}² are the mean and variance of the batch, and ϵ is a small constant for numerical stability.

Unlike Batch Normalization, Layer Normalization normalizes the inputs across the features instead of the batch dimension. This is particularly effective for recurrent neural networks and situations where the batch size is small.

Lastly, to prevent overfitting and ensure the model's generalizability, regularization techniques like L1/L2 regularization, Dropout, and Early Stopping are employed. L1/L2 Regularization add a penalty to the loss function. L1 regularization (Lasso) adds the absolute value of the coefficients as penalty, while L2 regularization (Ridge) adds the square of the coefficients. Dropout randomly deactivates a fraction of neurons during training, forcing the network to learn redundant representations and improving its generalization ability. Early stopping monitors the model's performance on a validation set and stops training when the performance starts to degrade, effectively preventing overfitting. By integrating these sophisticated optimizers, normalization, and regularization techniques, the model achieves efficient training, stable convergence, and maintains robustness when generalized to new data.

The embodiments of the invention in which an exclusive property or privilege is claimed are defined as follows:

1. A method for altering topology of terrain, incorporating a method for predicting the topological deformation of a land surface due to subterranean slurry injection incorporating:
a. Geospatial mesh computation using data extracted from one or more of the following mechanisms:
i. Land Survey
ii. Electrical Resistivity Tomography
iii. Ground Penetrating Radar
iv. Seismic Refraction
v. Reflection Surveys
vi. Electromagnetic Surveys
vii. Gravimetric Surveys
viii. Geotechnical Drilling
ix. Soil Sampling
x. Geocore Sampling
xi. Topsoil Testing
xii. Vegetation Analysis
xiii. Property Valuations
b. Geospatial mesh computation, utilizing one or more of the following methodologies with weighted contribution coefficients from the inputs in a):
i. Interpolation techniques
ii. Learning techniques
c. Topological deformation computation, utilizing one or more of the following methodologies:
i. CFD/FEA time step simulations
ii. Reinforcement learning-based approaches
iii. Supervised learning-based approaches
iv. Rule-based approximations
v. Geometric simplification approaches—aperture symmetry assumptions,
d. The aforementioned methods constitute in part or full a method which output one or more of the following:
i. An actionable set of guidelines for the execution of surface topology alteration through subterranean slurry injection, which may be augmented by surface grading, extraction, or fill
ii. Predicted resulting geospatial meshes
iii. Predicted flood statistics
iv. Flatness coefficients
v. Water pooling hotspot predictions
vi. Updated seismic data
vii. Predicted sequestration-related data
e. The aforementioned methods are refined through collected data pertaining to one or more of the following:
i. Pre-specified guidelines
ii. Guidelines concurrently modified or generated alongside injection procedures
iii. Sensor data associated with geospatial mesh states.

2. The method of claim 1 wherein a batched or continuous feedback loop is established, enabling the predictive and control models to be fine-tuned using data extracted from historical real, simulated subterranean injection trials, or a combination of the two.

3. The method of claim 1 wherein machine learning-based methods are used to simulate geospatial deformation resulting from parameterized injection profiles.

4. The method of claim 1 wherein computational fluid dynamics-based methods are used in conjunction with finite element analysis to predict time sequences of geospatial deformation resulting from parameterized injection profiles.

5. The method of claim 1 wherein the predictive modeling capability is used to optimize injection parameters such as drilling locations, depths, orientations, aperture-specific slurry compositions, time-dynamic slurry compositions, multi-hole injection scheduling, duration of the use of initial fracking fluid, time series of optimal topographies, and/or the duration of the use of flushing fluid, thereby comprising a comprehensive site plan.

6. The method of claim 4 wherein the generated site plan is taken as input to the real-time control system as time series-associated guidelines on system behavior.

7. A method for altering topology of terrain, incorporating a method for the determination of slurry injection parameters, incorporating:
a. Site-specific context, utilizing one or more of the following details:
i. Application—elevation, sequestration
ii. Available additives—specific dimensioned wood chips, guar gum
iii. Desired result—topological deformation, flood risk decrease, flattening, amount to be sequestered
b. Procedural characteristics, utilizing one or more of the following data:
i. Geospatial Topography
ii. System State, comprised by one or more of the following:
1. Slurry composition
2. Aperture jetting
3. Slurry movement
4. Variable Frequency Drive (VFD) values
5. Flow rates
6. Dynamic ground anchor states
7. Pressurized air/mixture states in central and auxiliary holes
8. Well locations
c. Resulting in slurry injection site plans, adhering to one or more of the following categories:
i. Static—the injection parameters do not change throughout the duration of the injection procedure
ii. Time-dynamic—the injection parameters change throughout the duration of the injection procedure, as determined by time-series specified profiles
iii. Event-dynamic—the injection parameters change throughout the duration of the injection procedure, as determined by event-series specified profiles.

8. The method of claim 7 wherein machine learning-based approaches are used to inform decisions about the static or dynamic composition of slurry to attain specific topographies or maximize storage of atmospheric carbon in the form of solid biomass under geospatial and hardware constraints.

9. A method for altering topology of terrain, incorporating a method of automated real-time modification of slurry injection parameters, incorporating:
a. Data streams comprised of one or more of the following:
i. Injection simulation inferences
ii. Sensor data
iii. Geospatial mesh approximations
b. Informed by static data contained in one or more of the following:
i. Time series data of previous injections
ii. Event series data of previous injections
iii. Slurry injection site plans
c. Control of system state to achieve one or more of the following objectives:
i. Matching of desired intermediate or final geospatial mesh
ii. Matching of desired system state
d. Computation of one or more of the following:
i. Feasibility of system state changes
ii. Current geospatial meshes
iii. Predicted topological alteration.

10. The method of claim 3 wherein the system aims to control storage and mix tank fill levels, detect and handle clogging events, prevent ground rupturing events, attain specific geospatial topographies, open subterranean aperture spaces, control dynamic ground anchors, control pressurized water jets, manipulate aperture-localized pressure, slurry movement directionality, and/or decommission holes.

11. The method of claim 3 wherein the system references time series associated topographies and infers using live sensor data streams optimal injection system states.

12. The method of claim 3 wherein a batched or continuous feedback loop is established, enabling the predictive and control models to be fine-tuned using data extracted from historical real, simulated subterranean injection trials, or a combination of the two.
